(19) United States
(12) Patent Application Publication
(10) Pub. No.: US 2024/0065550 A1
(43) Pub. Date: Feb. 29, 2024

(54) Method or System Using Machine Learning and/or Artificial Intelligence (AI) to Control the Operation of an Implanted Medical Device Based on Biometric Indicators

(71) Applicant: Robert A. Connor, St. Paul, MN (US)

(72) Inventor: Robert A. Connor, St. Paul, MN (US)

(73) Assignee: Medibotics LLC, St. Paul, MN (US)

(21) Appl. No.: 18/386,790

(22) Filed: Nov. 3, 2023

Related U.S. Application Data
(63) Continuation-in-part of application No. 17/410,297, filed on Aug. 24, 2021.
Application No. 17/410,297 is a continuation-in-part of application No. 16/598,514, filed on Oct. 10, 2019, now abandoned.
Application No. 16/598,514 is a continuation-in-part of application No. 16/568,580, filed on Sep. 12, 2019, now Pat. No. 11,478,158.
Application No. 16/568,580 is a continuation-in-part of application No. 16/150,469, filed on Oct. 3, 2018, now abandoned.
Application No. 16/150,469 is a continuation-in-part of application No. 15/418,620, filed on Jan. 27, 2017, now abandoned.
Application No. 15/418,620 is a continuation-in-part of application No. 14/459,937, filed on Aug. 14, 2014, now abandoned.
Application No. 14/459,937 is a continuation-in-part of application No. 14/951,475, filed on Nov. 24, 2015, now Pat. No. 10,314,492.
Application No. 14/951,475 is a continuation-in-part of application No. 14/623,337, filed on Feb. 16, 2015, now Pat. No. 9,582,035.
Application No. 14/623,337 is a continuation-in-part of application No. 14/071,112, filed on Nov. 4, 2013, now abandoned.
Application No. 14/071,112 is a continuation-in-part of application No. 13/901,131, filed on May 23, 2013, now Pat. No. 9,536,449.
(60) Provisional application No. 62/857,942, filed on Jun. 6, 2019.
(60) Provisional application No. 62/857,942, filed on Jun. 6, 2019.
(60) Provisional application No. 62/439,147, filed on Dec. 26, 2016.
(60) Provisional application No. 62/297,827, filed on Feb. 20, 2016.
(60) Provisional application No. 62/245,311, filed on Oct. 23, 2015.
(60) Provisional application No. 61/866,583, filed on Aug. 16, 2013.

(51) Int. Cl. A61B 5/00

(52) U.S. Cl. CPC A61B 5/0031; A61B 5/6801; A61B 5/7264

(57) ABSTRACT

This invention is a method or system which uses machine learning and/or artificial intelligence (AI) to adjust, manage, and/or control the operation of one or more implanted medical devices. This method or system identifies the lagged effects of operating parameters of the one or more implanted medical devices on a person's biometric indicators and then adjusts the operating parameters of the one or more implanted medical devices to change the values of the biometric indicators in a desired direction and/or by a desired amount.

### CROSS-REFERENCE TO RELATED APPLICATIONS

This application is a continuation in part of U.S. patent application Ser. No. 17/410,297 filed on 2021 Aug. 24. U.S. patent application Ser. No. 17/410,297 was a continuation in part of U.S. patent application Ser. No. 16/598,514 filed on 2019 Oct. 10. U.S. patent application Ser. No. 17/410,297 was also a continuation in part of U.S. patent application Ser. No. 16/568,580 filed on 2019 Sep. 12. U.S. patent application Ser. No. 17/410,297 was also a continuation in part of U.S. patent application Ser. No. 16/150,469 filed on 2018 Oct. 3.

U.S. patent application Ser. No. 16/598,514 claimed the priority benefit of U.S. provisional patent application 62/857,942 filed on 2019 Jun. 6. U.S. patent application Ser. No. 16/568,580 claimed the priority benefit of U.S. provisional patent application 62/857,942 filed on 2019 Jun. 6. U.S. patent application Ser. No. 16/150,469 was a continuation in part of U.S. patent application Ser. No. 15/418,620 filed on 2017 Jan. 27. U.S. patent application Ser. No. 16/150,469 was a continuation in part of U.S. patent application Ser. No. 14/459,937 filed on 2014 Aug. 14.

U.S. patent application Ser. No. 15/418,620 claimed the priority benefit of U.S. provisional patent application 62/439,147 filed on 2016 Dec. 26. U.S. patent application Ser. No. 15/418,620 claimed the priority benefit of U.S. provisional patent application 62/297,827 filed on 2016 Feb. 20. U.S. patent application Ser. No. 15/418,620 was a continuation in part of U.S. patent application Ser. No. 14/951,475 filed on 2015 Nov. 24 which issued as U.S. patent Ser. No. 10/314,492.

U.S. patent application Ser. No. 14/951,475 claimed the priority benefit of U.S. provisional patent application 62/245,311 filed on 2015 Oct. 23. U.S. patent application Ser. No. 14/951,475 was a continuation in part of U.S. patent application Ser. No. 14/623,337 filed on 2015 Feb. 16 which issued as U.S. Pat. No. 9,582,035. U.S. patent application Ser. No. 14/951,475 was a continuation in part of U.S. patent application Ser. No. 14/071,112 filed on 2013 Nov. 4. U.S. patent application Ser. No. 14/951,475 was a continuation in part of U.S. patent application Ser. No. 13/901,131 filed on 2013 May 23 which issued as U.S. Pat. No. 9,536,449. U.S. patent application Ser. No. 14/459,937 claimed the priority benefit of U.S. provisional patent application 61/866,583 filed on 2013 Aug. 16.

The entire contents of these related applications are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to methods and systems for human circulatory assistance.

### INTRODUCTION

Proper cardiovascular functioning, including good blood circulation and tissue oxygenation, is important for human physiological functioning and tissue health. When cardiovascular functioning is significantly impaired, a cardiac device such as a pacemaker (to assist in cardiac pacing) or a ventricular assist device (to assist in blood pumping) can be implanted to improve cardiovascular functioning. A person's cardiovascular needs vary over time based on the person's activity level, as well as physiological and anatomical changes over time. Accordingly, it is important that the operation of an implanted cardiac device be automatically adjusted to respond to these changing needs.

However, many cardiovascular problems first appear in the extremities of a person's body, such as the person's feet and hands. Biometric information from those extremities is needed for accurate adjustment and optimization of the operation of a cardiac device, but this information is not available from sensors in a cardiac device which is implanted in a person's torso. This is the unmet clinical need which is addressed by this invention. A method or system for assisting human cardiovascular functioning which adjusts and optimizes the operation of an implanted cardiac device based on biometric information from body extremities can help to manage chronic heart conditions, improve tissue health (particularly in extremities such as feet and hands), promote wound healing, and potentially even avoid amputation.

### REVIEW OF THE RELEVANT ART

U.S. patent application 20040131998 (Marom et al., Jul. 8, 2004, “Cerebral Programming”) and U.S. Pat. No. 7,499,894 (Marom et al., Mar. 3, 2009, “Cerebral Programming”) disclose training a biological neural network to control a pacemaker. U.S. patent applications 20050081847 (Lee et al., Apr. 21, 2005, Automatic Activation of Medical Processes”) and 20100106211 (Lee et al., Apr. 29, 2010, “Automatic Activation of Medical Processes”), as well as U.S. Pat. No. 7,668,591 (Lee et al., Feb. 23, 2010, “Automatic Activation of Medical Processes”) and U.S. Pat. No. 8,380,296 (Lee et al., Feb. 19, 2013, “Automatic Activation of Medical Processes”) disclose automatic modification of therapies or other medical processes based on brain state.

U.S. patent applications 20050115561 (Stahmann et al., Jun. 2, 2005, “Patient Monitoring, Diagnosis, and/or Therapy Systems and Methods”) and 20110061647 (Stahmann et al., Mar. 17, 2011, “Patient Monitoring, Diagnosis, and/or Therapy Systems and Methods”), as well as U.S. Pat. No. 7,787,946 (Stahmann et al., Aug. 31, 2010, “Patient Monitoring, Diagnosis, and/or Therapy Systems and Methods”), disclose systems and methods involving an implantable device configured to perform at least one cardiac-related function, a patient-external respiratory therapy device, and a communication channel configured to facilitate communication between the implantable device and the respiratory therapy device. U.S. patent application 20060195039 (Drew et al, Aug. 31, 2006, “Clustering with Combined Physiological Signals”) and U.S. Pat. No. 8,768,446 (Drew et al., Jul. 1, 2014, “Clustering with Combined Physiological Signals”) disclose a method of generating an extended cluster by an implanted medical device.

U.S. patent applications 20070260286 (Giftakis et al., Nov. 8, 2007, “System and Method for Utilizing Brain State Information to Modulate Cardiac Therapy”) and U.S. patent application 20070265677 (Giftakis et al., Nov. 15, 2007, “System and Method for Utilizing Brain State Information to Modulate Cardiac Therapy”) and U.S. Pat. No. 8,209,019 (Giftakis et al., Jun. 26, 2012, “System and Method for Utilizing Brain State Information to Modulate Cardiac Therapy”) and U.S. Pat. No. 8,214,035 (Giftakis et al., Jul. 3, 2012, “System and Method for Utilizing Brain State Information to Modulate Cardiac Therapy”) disclose a system for regulating or modulating cardiac therapy using brain state information.

U.S. Pat. No. 7,313,440 (Miesel, Dec. 25, 2007, “Collecting Posture and Activity Information to Evaluate Therapy”) discloses using posture to set therapy parameters for a medical device. U.S. Pat. No. 8,090,432 (Cinbis et al., Jan. 3, 2012, “Implantable Tissue Perfusion Sensing System and Method”) and U.S. Pat. No. 8,165,662 (Cinbis et al., Apr. 24, 2012, “Implantable Tissue Perfusion Sensing System and Method”) disclose using electrodes to sense cardiac signals to identify a cardiac event.

U.S. Pat. No. 8,108,038 (Giftakis et al., Jan. 31, 2012, “System and Method for Segmenting a Cardiac Signal Based on Brain Activity”) and U.S. Pat. No. 8,209,009 (Giftakis et al., Jun. 26, 2012, “System and Method for Segmenting a Cardiac Signal Based on Brain Stimulation”) disclose a medical device system with a brain monitoring element, cardiac monitoring element and a processor. U.S. Pat. No. 8,112,148 (Giftakis et al., Feb. 7, 2012, “System and Method for Monitoring Cardiac Signal Activity in Patients with Nervous System Disorders”) discloses a medical device system and method for monitoring cardiac signal activity in patients with nervous system disorders.

U.S. Pat. No. 8,428,729 (Schwartz et al., Apr. 23, 2013, “Cardiac Stimulation Apparatus and Method for the Control of Hypertension”) discloses a method that electrically stimulates a heart muscle to alter the ejection profile of the heart, to control the mechanical function of the heart and reduce the observed blood pressure of the patient. U.S. Pat. No. 8,463,345 (Kuhn et al., Jun. 11, 2013, “Device and Method for Monitoring of Absolute Oxygen Saturation and Total Hemoglobin Concentration”), U.S. Pat. No. 8,634,890 (Kuhn et al., Jan. 21, 2014, “Device and Method for Monitoring of Absolute Oxygen Saturation and Tissue Hemoglobin Concentration”) and U.S. Pat. No. 8,666,466 (Kuhn et al., Mar. 4, 2014, “Device and Method for Monitoring of Absolute Oxygen Saturation and Tissue Hemoglobin Concentration”) disclose an implantable oxygen saturation monitor.

U.S. Pat. No. 8,515,548 (Rofougaran et al., Aug. 20, 2013, “Article of Clothing Including Bio-Medical Units”) discloses an article of clothing with a plurality of bio-medical units integrated into the clothing fabric. U.S. Pat. No. 8,617,082 (Zhang et al., Dec. 13, 2013, “Heart Sounds-Based Pacing Optimization”) discloses an implantable medical device that receives both heart sounds and electrogram signals. U.S. Pat. No. 8,634,890 (Kuhn et al., Jan. 21, 2014, “Device and Method for Monitoring of Absolute Oxygen Saturation and Tissue Hemoglobin Concentration”) discloses a method and device for measuring tissue oxygen level.

U.S. patent application 20150246166 (Greatrex et al., Sep. 3, 2015, “Ventricular Assist Device and Method of Controlling Same”) discloses a method of controlling the speed of a ventricular assist device, in particular the rotational speed of a rotary blood pump, wherein at least temporarily the speed of the device is modulated around a mean speed and a response of the native heart to this modulation is measured. U.S. Pat. No. 9,131,865 (Thompson-Nauman et al., Sep. 15, 2015, “Method and Apparatus for Cardiac Function Monitoring”) and U.S. Pat. No. 9,138,157 (Thompson-Nauman et al., Sep. 22, 2015, “Method and Apparatus for Cardiac Function Monitoring”) disclose a method and medical device for monitoring cardiac function in a patient that includes a plurality of electrodes to deliver cardiac pacing therapy.

U.S. Pat. No. 9,283,341 (Ujhazy et al., Mar. 15, 2016, “Methods and Apparatus for Heart Failure Treatment”) discloses methods and an apparatus for assessing the condition of and treating patients for heart failure by the delivery of continuous positive airway pressure. U.S. Pat. No. 9,307,907 (Condurso et al., Apr. 12, 2016, “System and Method for Dynamically Adjusting Patient Therapy”) discloses a system and method of managing therapy that monitors all aspects of the medication delivery to a patient. U.S. Pat. No. 9,320,443 (Libbus et al., Apr. 26, 2016, “Multi-Sensor Patient Monitor to Detect Impending Cardiac Decompensation”) discloses a system for detecting impending acute cardiac decompensation.

U.S. patent application 20160143533 (Keenan et al., May 26, 2016, “Medical Device System Having an Implanted Medical Device and an External Device”) discloses an external device for wireless monitoring of an implanted device. U.S. patent application 20160228627 (Wiesener et al., Aug. 11, 2016, “Blood Pump Control System and Method for Controlling a Blood Pump”) discloses methods for controlling the speed of a pump based on a valve state index and/or for deriving a valve state from time-series signal representing a pressure difference or a flow rate. U.S. patent application 20180126053 (Zilbershlag, May 10, 2018, “Wristwatch for Monitoring Operation of an Implanted Ventricular Assist Device”) discloses a wristwatch wirelessly connected to an implanted medical device such as a VAD to monitors the operation of the VAD.

U.S. patent Ser. No. 10/010,662 (Wiesener et al., Jul. 3, 2018, “Blood Pump Control System and Method for Controlling a Blood Pump”) discloses methods for controlling the speed of a blood pump based on a valve state index. U.S. patent Ser. No. 10/258,798 (Panken et al., Apr. 6, 2019, “Patient Directed Therapy Control”) discloses patient control of therapy by changing their mental activity. U.S. patent application 20190298987 (Freeman et al., Oct. 3, 2019, “Garments for Wearable Cardiac Monitoring and Treatment Devices”) discloses a garment with ECG monitors.

U.S. patent Ser. No. 10/485,978 (Thakur et al., Nov. 26, 2019, “Pacing Site and Configuration Optimization Using a Combination of Electrical and Mechanical Information”) discloses using heart sounds to evaluate alternative cardiac pacing sites. U.S. patent application 20200179706 (Thakur et al., Jun. 11, 2020, “Hemodynamically Optimized Rate Response Pacing Using Heart Sounds”) disclose san apparatus comprising a stimulus circuit configured to deliver electrical pacing therapy to a subject when operatively coupled to a plurality of electrodes, including a heart sound sensing circuit.

U.S. patent application 20200215246 (Tal et al., Jul. 9, 2020, “Method and System for Ventricular Assistive Device Adjustment Using a Wearable Device”) discloses control of a VAD device using wearable sensors, such as blood pressure sensors. U.S. patent application 20210000349 (Goetz, Jan. 7, 2021, “Remote Titration of Therapy Delivered by an Implantable Medical Device”) discloses techniques for remotely titrating a therapy delivered using an implantable medical device system. U.S. patent application 20210177335 (Varadan et al., Jun. 17, 2021, “Wearable Congestive Heart Failure Management System”) discloses a non-invasive, wearable and portable medical device for evaluation and monitoring the heart condition for patients with congestive heart failure.

U.S. patent application 20210228886 (Pronovici et al., Jul. 29, 2021, “Adjustment of Mechanical Motion Sensing for Controlling Cardiac Pacing”) discloses sensing the mechanical motion of a heart for cardiac pacing therapy. U.S. patent application 20210236801 (Sarkisyan et al., Aug. 5, 2021, “Integrated Adjustable Multi-Pump Mechanical Circulatory Support Device”) discloses a mechanical circulatory support device with first and second blood pumps. U.S. patent application 20210252277 (Webster et al., Aug. 19, 2021, “Wearable Medical Device With Integrated Blood or tissue oxygen Saturation Level Device”) discloses a wearable medical device (WMD) with a blood or tissue oxygen level sensor.

U.S. patent application 20210298658 (Ghosh, Sep. 30, 2021, “Pacing Efficacy Determination Using a Representative Morphology of External Cardiac Signals”) discloses systems and methods for detecting the efficiency of cardiac pacing using external cardiac signals. U.S. patent application 20210315521 (Webster et al., Oct. 14, 2021, “Modular Cardiac Patient Treatment and Monitoring”) discloses a wearable healthcare system having a reconfigurable medical device. U.S. patent application 20220040487 (Burnam et al., Feb. 10, 2022, “An Intelligently, Continuously, and Physiologically Controlled Pacemaker and Method of Operation of the Same”) discloses a pacemaker control system with internal and external sensors.

U.S. patent application 20220072316 (Yoon et al., Mar. 10, 2022, “Dual Sensors to Control Pacing Rate”) discloses a medical device which uses acceleration and temperature to adjust the rate of cardiac pacing. U.S. patent application 20220088369 (Chopra et al., Mar. 24, 2022, “Systems and Methods for Pump-Assisted Blood Circulation”) discloses systems, devices, and methods that use a pump to assist blood flow.

U.S. patent application 20220152397 (Kelley et al., May 19, 2022, “Neurostimulation Evaluation, Programming and Control Based on Sensed Blood Flow”) discloses a neurostimulation device that receives blood flow information.

U.S. patent application 20220160250 (Anderson et al., May 26, 2022, “Detection and Mitigation of Inaccurate Sensing by an Implanted Sensor of a Medical System”) discloses techniques for detecting and mitigating inaccurate sensing in a medical system. U.S. patent application 20220168576 (Ghosh, Jun. 2, 2022, “Evaluation and Adjustment of Left Bundle Branch (LBB) Pacing Therapy”) discloses systems and methods for evaluation and adjustment of left bundle branch pacing therapy. U.S. patent application 20220192605 (Webster et al., Jun. 23, 2022, “Managing Cardiac Risk Based on Physiological Markers”) discloses a method to track the cardiac health of a patient using a motion sensor.

U.S. patent application 20220211282 (Rowland et al., Jul. 7, 2022, “Hemodynamic Monitoring System and Method and Harness for Same”) discloses a harness-based hemodynamic monitoring system. U.S. patent application 20220211332 (Demmer et al., Jul. 7, 2022, “Medical Device System for Monitoring Patient Health”) discloses a method of monitoring a patient with a medical device and a peripheral device. U.S. patent application 20220225949 (Umberger et al., Jul. 21, 2022, “Wearable Device Network System”) discloses a method to track cardiac health using a sensor. U.S. patent application 20220240832 (Kim et al., Aug. 4, 2022, “Wearable Medical Device with Zoneless Arrhythmia Detection”) discloses a wearable monitoring device to zonelessly detect arrhythmias from physiological signals.

U.S. patent application 20220265209 (Panken et al., Aug. 25, 2022, “Medical Device Using Spectral Activity Processing”) discloses a stimulation system with accelerometers. U.S. patent application 20220280047 (Stadler et al., Sep. 8, 2022, “Automatic Alert Control for Acute Health Event”) discloses an antenna to wirelessly receive communication from a medical device. U.S. patent application 20220296906 (Westphal et al., Sep. 22, 2022, “Indirect Sensing Mechanism for Cardiac Monitoring”) discloses systems and techniques with a mechanosensor for detecting changes in patient health. U.S. patent application 20220314013 (Sullivan, Oct. 6, 2022, “Wearable Cardioverter Defibrillator (WCD) System Making Shock/No Shock Determinations From Multiple Patient Parameters”) discloses a wearable cardioverter defibrillator (WCD) system which senses patient parameters from different parts of a body.

U.S. patent application 20220323012 (Pool et al., Oct. 13, 2022, “Sensor Integration in Cardiac Implant Devices”) discloses a sensor-retention structure with a sensor-support strut. U.S. patent application 20220369937 (Cho et al., Nov. 24, 2022, “Acute Health Event Monitoring”) discloses a system to detect sudden cardiac arrest. U.S. patent application 20230046704 (Yoder et al., Feb. 16, 2023, “Remote Monitoring and Support of Medical Devices”) discloses systems and techniques for detecting a change in patient health and modifying settings of a medical device. U.S. patent application 20230077146 (Muse et al., Mar. 9, 2023, “Artificial Heart Control Systems and Methods”) discloses a controller for an artificial heart for activity-specific adjustments to the operation of the artificial heart by obtaining sensor data.

U.S. patent application 20230114876 (Brincat et al., Apr. 13, 2023, “Body Area Network Having Sensing Capability”) discloses systems and methods for monitoring health of a user including a plurality of sensors worn by or implanted in a user. U.S. patent application 20230138882 (Webster et al., May 4, 2023, “Integrative Wearable Health Monitoring”) discloses an integrative wearable health monitoring capable of displaying information about devices and sensors. U.S. patent application 20230149694 (Taskin et al., May 18, 2023, “Mechanical Circulatory Support Device”) discloses a pump which provides pulsating blood flow.

U.S. patent application 20230154304 (Ship et al., May 18, 2023, “Systems and Methods for Activity Tracking and Physiological Sensing for Cardiac Recovery Assessment and Support in Mechanical Blood Pumps”) discloses a system and method for supporting and monitoring mechanical blood pumps using input from the one or more sensors. U.S. patent application 20230181916 (Zhang et al., Jun. 15, 2023, “Modular Defibrilator System”) discloses an implantable cardiac monitoring device (ICMD) which monitors physiological signals and, in response to detecting a current or imminent arrhythmia in the patient, transmits data to an external user device.

U.S. patent application 20230277100 (Meeker et al., Sep. 7, 2023, “Oximetry Monitoring in a Wearable Medical Device”) discloses technologies and implementations for oximetry monitoring in a wearable medical device (WMD). U.S. patent application 20230285764 (Rowbotham et al., Sep. 14, 2023, “Wearable Medical System (WMS) Implementing Wearable Cardioverter Defibrillator (WCD) and Recording ECG of Patient in Regular Mode and in Rich Mode”) discloses a wearable medical system (WMS) with a wearable cardioverter defibrillator (WCD) that senses ECG signals. U.S. patent application 20230309861 (Galarneau et al., Oct. 5, 2023, “Motion Sensor-Modulated Cardiac Episode Detection and/or Alerting”) discloses a medical device system which stores information relating to a cardiac event.

### SUMMARY OF THE INVENTION

This invention is a method or system for controlling (e.g. adjusting or managing) the operation of one or more implanted medical devices. This method or system uses machine learning and/or artificial intelligence (AI) to identify the lagged effects of operating parameters of the one or more implanted medical devices on a person's biometric indicators. This method or system then adjusts the operating parameters of the one or more implanted medical devices to change the values of the biometric indicators in a desired direction and/or by a desired amount. In an example, an implanted medical device can be a cardiac pacemaker. In an example, biometric indicators can include blood or tissue oxygenation level and peripheral blood pressure.

In an example, values of a person's biometric indicators can be obtained from a combination of internal (e.g. implanted) sensors and external (e.g. wearable) sensors. In an example, values of biometric indicators can be obtained from sensors at different locations in and/or on a person's body. Controlling the operation of one or more implanted medical devices based on biometric indicator values from different locations can help to ensure the tissue health of body extremities better than only using values from a sensor in a single location (e.g. only a sensor in a single implanted medical device). For example, there can be variation in blood or tissue oxygenation levels and peripheral blood pressures between different body extremities. A method or system which recognizes and responds to this variation can adjust the operation of one or more implanted medical devices more effectively to maintain good tissue health for all body extremities.

### INTRODUCTION TO THE FIGURES

FIG. 1 shows a method of managing the operation of an implanted medical device comprising: using machine learning and/or artificial intelligence to identify the effects of operating parameters of an implanted medical device on biometric indicators during a first time period; and using those identified effects to adjust the operating parameters of the implanted medical device to change the biometric indicators during a second time period.

FIG. 2 shows an example of a method of managing the operation of an implanted medical device comprising: receiving data during a first time period concerning operating parameters of an implanted medical device; receiving data during a second time period concerning biometric indicators; using machine learning and/or artificial intelligence to identify lagged effects of the operating parameters of the implanted medical device on the biometric indicators; receiving data during a third time period concerning the biometric indicators; and using the previously-identified lagged effects to determine how to change the operating parameters of the implanted medical device in order to change the biometric indicators in a desired direction and/or by a desired amount.

### DETAILED DESCRIPTION OF THE FIGURES

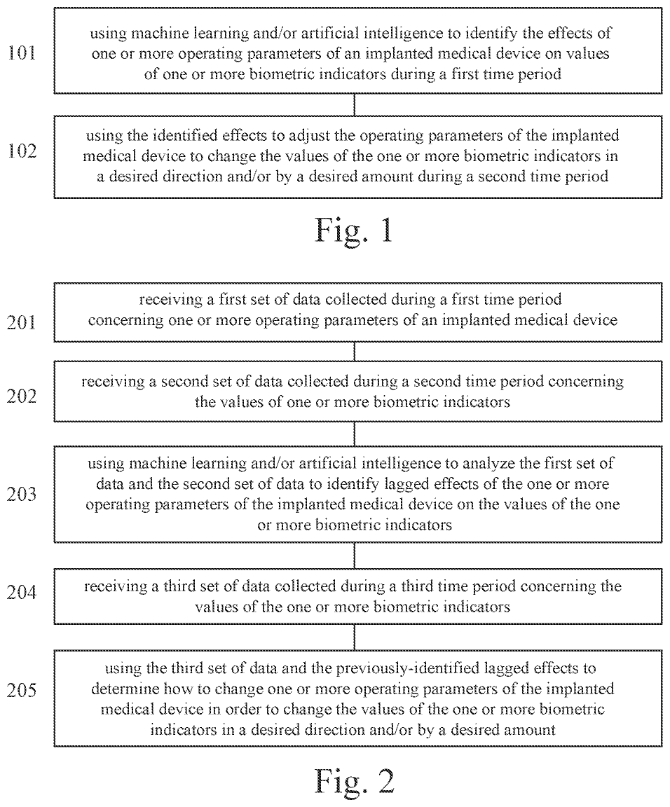

FIG. 1 shows an example of a method of managing the operation of an implanted medical device comprising: using machine learning and/or artificial intelligence to identify the effects of one or more operating parameters of an implanted medical device implanted in a person on values of one or more biometric indicators concerning the person during a first time period 101; and using the identified effects to adjust the operating parameters of the implanted medical device to change the values of the one or more biometric indicators in a desired direction and/or by a desired amount during a second time period 102.

FIG. 2 shows an example of a method of managing the operation of an implanted medical device comprising: receiving a first set of data collected during a first time period concerning one or more operating parameters of an implanted medical device which is configured to be implanted in a person 201; receiving a second set of data collected during a second time period concerning the values of one or more biometric indicators for the person 202; using machine learning and/or artificial intelligence to analyze the first set of data and the second set of data to identify lagged effects of the one or more operating parameters of the implanted medical device on the values of the one or more biometric indicators 203; receiving a third set of data collected during a third time period concerning the values of the one or more biometric indicators for the person 204; and using the third set of data and the previously-identified lagged effects to determine how to change one or more operating parameters of the implanted medical device in order to change the values of the one or more biometric indicators in a desired direction and/or by a desired amount 205.

In an example, a method of managing the operation of an implanted medical device can comprise: using machine learning and/or artificial intelligence to identify the effects of one or more operating parameters of an implanted medical device implanted in a person on values of one or more biometric indicators concerning the person during a first time period; and using the identified effects to adjust the operating parameters of the implanted medical device to change the values of the one or more biometric indicators in a desired direction and/or by a desired amount during a second time period.

In an example, the implanted medical device can be selected from the group consisting of: implanted cardiac pacemaker; implanted cardioverter defibrillator; implanted blood pump; implanted neurostimulator; and implanted drug pump. In an example, the one or more biometric indicators can be selected from the group consisting of: blood or tissue oxygen level; blood pressure; heart rate; blood glucose level; respiration rate; body posture; body motion; body temperature; chewing or swallowing motion; and brainwave pattern. In an example, data on the one or more biometric indicators can come from one or more sensors which are selected from the group consisting of: optical sensor; spectroscopic optical sensor; oximeter; PPG sensor; glucose sensor; inertial motion sensor; strain sensor; moisture sensor; electromagnetic energy sensor; EEG sensor; and EMG sensor.

In an example, at least some of the data on the one or more biometric indicators can come from one or more sensors on one or more wearable devices what are selected from the group consisting of: smart watch or watch band; wrist band; arm band; bracelet; finger ring; necklace; smart eyewear or an eyewear attachment; smart ear bud or ear ring; smart clothing; and sensor-enabled adhesive patch. In an example, the operating parameters of the implanted medical device can be adjusted based at least in part on the values of the one or more biometric indicators during the second time period. In an example, the operating parameters of the implanted medical device can be adjusted based at least in part on changes overtime and/or variation over time in the values of the one or more biometric indicators during the second time period. In an example, data can come at least partly from a plurality of wearable devices which are worn at different locations on the person's body. In an example, the operating parameters of the implanted medical device can be adjusted based at least in part on differences in values for the one or more biometric indicators between sensors in wearable devices at different locations on the person's body.

In an example, a method of managing the operation of an implanted medical device can comprise: receiving a first set of data collected during a first time period concerning one or more operating parameters of an implanted medical device which is configured to be implanted in a person; receiving a second set of data collected during a second time period concerning the values of one or more biometric indicators for the person; using machine learning and/or artificial intelligence to analyze the first set of data and the second set of data to identify lagged effects of the one or more operating parameters of the implanted medical device on the values of the one or more biometric indicators; receiving a third set of data collected during a third time period concerning the values of the one or more biometric indicators for the person; and using the third set of data and the previously-identified lagged effects to determine how to change one or more operating parameters of the implanted medical device in order to change the values of the one or more biometric indicators in a desired direction and/or by a desired amount.

In an example, the implanted medical device can be selected from the group consisting of: implanted cardiac pacemaker; implanted cardioverter defibrillator; implanted blood pump; implanted neurostimulator; and implanted drug pump. In an example, the one or more biometric indicators can be selected from the group consisting of: blood or tissue oxygen level; blood pressure; heart rate; blood glucose level; respiration rate; body posture; body motion; body temperature; chewing or swallowing motion; and brainwave pattern. In an example, data on the one or more biometric indicators can come from one or more sensors which are selected from the group consisting of: optical sensor; spectroscopic optical sensor; oximeter; PPG sensor; glucose sensor; inertial motion sensor; strain sensor; moisture sensor; electromagnetic energy sensor; EEG sensor; and EMG sensor.

In an example, at least some of the data on the one or more biometric indicators can come from one or more sensors on one or more wearable devices what are selected from the group consisting of: smart watch or watch band; wrist band; arm band; bracelet; finger ring; necklace; smart eyewear or an eyewear attachment; smart ear bud or ear ring; smart clothing; and sensor-enabled adhesive patch. In an example, the operating parameters of the implanted medical device can be changed based at least in part on the values of the one or more biometric indicators in the third set of data. In an example, the operating parameters of the implanted medical device can be changed based at least in part on changes over time and/or variation over time in the values of the one or more biometric indicators in the third set of data. In an example, machine learning and/or artificial intelligence can be used to analyze the first set of data and the second set of data to identify lagged effects of changes in the one or more operating parameters of the implanted medical device on changes in the values of the one or more biometric indicators.

In an example, the operating parameters of the implanted medical device can be changed based at least in part on the whether the values of the one or more biometric indicators are outside their normal ranges for the person in the third set of data. In an example, the second set of data can be at least partly from a plurality of wearable devices which are worn at different locations on the person's body. In an example, the operating parameters of the implanted medical device can be changed based at least in part on differences in values for the one or more biometric indicators between sensors in wearable devices at different locations on the person's body.

A method of adjusting operating parameters of an implanted medical device based on biometric data from implanted sensors and wearable sensors comprising: receiving device parameter data collected during a first time period, wherein the device parameter data comprises values for one or more operating parameters of an implanted medical device which is configured to be implanted in a person; receiving biometric data collected during a second time period, wherein the biometric data comes from a combination of implanted sensors which are configured to be implanted in the person and wearable sensors which are configured to be worn by the person, wherein the biometric data comprises values for one or more biometric statistics concerning the person, and wherein the second period time is after the first time period; using machine learning to analyze the device parameter data and the biometric data to identify lagged effects of the operating parameters of the implanted medical device during the first time period on the biometric statistics during the second time period; receiving biometric data collected during a third time period; using machine learning to identify whether biometric statistics measured during the third time period should be changed in a selected direction and/or by a selected amount; and using machine learning and the previously-identified lagged effects to determine how the operating parameters of the implanted medical device should be changed in order to change the biometric statistics in the selected direction and/or by the selected amount during a fourth time period.

In an example, a wearable device which is part of a system or method can comprise a pulse oximeter. In an example, the operating parameters of an implanted cardiac pacemaker can be automatically adjusted based on peripheral oxygen saturation (SpO2) level or arterial oxygen saturation (SaO2) level as measured by external (e.g. wearable) and/or internal (e.g. implanted) oximeters. In an example, data concerning one or more biometric indicators can be obtained from a combination of internal (e.g. implanted) sensors and external (e.g. wearable) sensors on a pulse oximeter which is worn by the person. In an example, a closed-loop system for adjusting operating parameters of an implanted medical device can include an oxygen saturation sensor, SpO2 sensor, and/or pulse oximeter.

In an example, the operating parameters of an implanted cardiac pacemaker can be automatically adjusted based on machine learning (e.g. AI) and peripheral oxygen saturation (SpO2) level or arterial oxygen saturation (SaO2) level as measured by external (e.g. wearable) and/or internal (e.g. implanted) oxygen sensors. In an example, the operating parameters of an implanted medical device can be automatically adjusted based on peripheral oxygen saturation (SpO2) level or arterial oxygen saturation (SaO2) level as measured by external (e.g. wearable) and/or internal (e.g. implanted) oximeters. Working together in an integrated system, a wearable device for measuring oxygen level in body extremities and an implanted device for cardiac rhythm management can help to prevent oxygen deficiencies in body extremities. In an example, the operating parameters of an implanted cardiac pacemaker can be automatically adjusted based on blood or tissue oxygenation level as measured by external (e.g. wearable) and/or internal (e.g. implanted) oximeters.

In an example, the operating parameters of an implanted cardiac pacemaker can be automatically adjusted based on machine learning (e.g. AI) and blood or tissue oxygenation level as measured by external (e.g. wearable) and/or internal (e.g. implanted) oximeters. Without a wearable device component to measure body oxygen levels in body extremities, an implanted cardiac rhythm management device alone is not aware of oxygen deficiencies in body extremities. In an example, the operating parameters of an implanted medical device can be automatically adjusted based on blood or tissue oxygenation level as measured by external (e.g. wearable) and/or internal (e.g. implanted) oximeters. In an example, a method can comprise receiving biometric data from an oxygen saturation sensor, SpO2 sensor, and/or pulse oximeter.

Without an implanted cardiac rhythm management device, a wearable device alone can provide information on oxygenation levels in body extremities, but does not provide automatic therapeutic correction for oxygenation deficiency in body extremities. In an example, the operating parameters of an implanted cardiac pacemaker can be automatically adjusted based on machine learning (e.g. AI) and blood or tissue oxygenation level as measured by external (e.g. wearable) and/or internal (e.g. implanted) oxygen sensors. In an example, the operating parameters of an implanted cardiac pacemaker can be automatically adjusted based on blood or tissue oxygenation level as measured by external (e.g. wearable) and/or internal (e.g. implanted) oxygen sensors.

In an example, a closed loop system for human circulatory assistance can increase blood circulation by adjusting the operation of a cardiac pacemaker in response to a low body oxygenation level. In an example, the biometric indicator which is measured and managed by a system can be selected from the group consisting of: oxygenation level, carbon dioxide level, lactate or lactic acid level, blood pressure, heart rate variability, pulsatile blood volume, pulsatile blood lag, hydration level, respiration rate, exhaled gas composition, body glucose level, troponin level, body motion or exercise level, and sleep status or stage. In an example, human circulatory assistance can be provided by an implanted cardiac pacemaker whose operation is adjusted in real time based on overall body oxygenation level.

In an example, a cardiac pacemaker can deliver electromagnetic energy pulses to the heart via wires and/or leads. In an example, a cardiac pacemaker can be implanted within the heart, wherein it directly delivers electromagnetic energy pulses to the heart walls. In an example, the system can decrease the frequency and/or magnitude of electromagnetic pulses delivered to the person's heart when analysis of data from the light receiver indicates a high level of oxygen in body tissue (and/or fluid). In an example, the system can change the frequency and/or magnitude of electromagnetic pulses delivered to a person's heart when analysis of data from the light receiver indicates a change in the level of oxygen in body tissue (and/or fluid). In an example, an implanted circulatory assistance device can be a cardiac pacemaker which delivers periodic electromagnetic energy pulses to a person's heart in order to stimulate and/or regulate contraction of heart muscles.

In an example, the operating parameters of an implanted cardiac pacemaker can be automatically adjusted based on peripheral oxygen saturation (SpO2) level or arterial oxygen saturation (SaO2) level as measured by external (e.g. wearable) and/or internal (e.g. implanted) oxygen sensors. In an example, a method can comprise using a support vector machine (SVM) to adjust operating parameters of an implanted cardiac pacemaker based on biometric indicators measured by implanted and wearable sensors. In an example, the operating parameters of an implanted cardiac pacemaker which are adjusted by a system and/or method can comprise duty cycle, and/or left ventricular or thoracic surrogate electrical activation times. In an example, a system can monitor a person's systolic blood pressure and adjust the operating parameters of an implanted cardiac pacemaker in response to abnormal blood pressure values.

In an example, a method can comprise increasing the frequency and/or magnitude of electromagnetic pulses delivered from a pacemaker to a person's heart when analysis of data from an oxygen sensor indicates a low level of oxygen in blood and/or body tissue. In an example, a method can comprise increasing the frequency and/or magnitude of electromagnetic pulses delivered from a pacemaker to a person's heart when analysis of data from an implanted oxygen sensor indicates a low level of oxygen in blood and/or body tissue. In an example, a method can comprise increasing the frequency and/or magnitude of electromagnetic pulses delivered from a pacemaker to a person's heart when analysis of data from an external (e.g. wearable) oxygen sensor indicates a low level of oxygen in blood and/or body tissue.

In an example, the operating parameters of an implanted medical device can be automatically adjusted based on blood or tissue oxygenation level as measured by external (e.g. wearable) and/or internal (e.g. implanted) oxygen sensors. In an example, the operating parameters of an implanted cardiac pacemaker can be automatically adjusted based on blood or tissue oxygenation levels measured by external oxygen sensors worn on two different locations on a person's body. In an example, the operating parameters of a person's implanted cardiac pacemaker can be automatically adjusted based on combined analysis of blood or tissue oxygenation levels measured by oxygen sensors on the person's wrist and torso. In an example, the operating parameters of a person's implanted cardiac pacemaker can be automatically adjusted based on combined analysis of blood or tissue oxygenation levels measured by oxygen sensors on the person's wrist and leg. In an example, the operating parameters of a person's implanted cardiac pacemaker can be automatically adjusted based on combined analysis of blood or tissue oxygenation levels measured by oxygen sensors on the person's right and left wrists.

In an example, a method can comprise using artificial intelligence (AI) to adjust operating parameters of an implanted cardiac pacemaker based on biometric indicators measured by implanted and wearable sensors. In an example, the operating parameters of an implanted cardiac pacemaker can be automatically adjusted based on differences in the blood or tissue oxygenation levels which are measured by external oxygen sensors on two different locations on a person's body. In an example, the operating parameters of an implanted cardiac pacemaker can be automatically adjusted based on machine learning (e.g. AI) and peripheral oxygen saturation (SpO2) level or arterial oxygen saturation (SaO2) level as measured by external (e.g. wearable) and/or internal (e.g. implanted) oximeters.

In an example, the operation of an implanted non-central (peripheral) blood pump can be adjusted based on multivariate analysis of data concerning biometric indicator levels from a plurality of wearable devices worn at different locations on a person's body. In an example, the operation of an implanted non-central (peripheral) blood pump can be adjusted based on the average of biometric indicator levels, the lowest biometric indicator level, the highest biometric indicator level, and/or the range or variability of biometric indicator levels measured by a plurality of wearable devices worn at different locations on a person's body. In an example, the operating parameters of an implanted cardiac pacemaker can be automatically adjusted based on differences over time in the blood or tissue oxygenation levels which are measured by external oxygen sensors on two different locations on a person's body.

In an example, a method for adjustment and/or control of an implanted medical device (e.g. pacemaker, ICD, neurostimulator, or drug pump) can comprise: using machine learning and/or artificial intelligence to adjust one or more operating parameters of the implanted medical device based on one or more aspects (e.g. level, change rate, variation or cycle frequency over time, variation by body location, minimum value, or maximum value) of one or more biometric indicators (e.g. blood or tissue oxygen level, blood pressure, heart rate, respiration rate, body posture and/or movement, or blood glucose level) measured by a combination of implanted and wearable sensors.

In an example, a method for adjustment and/or control of an implanted medical device (e.g. pacemaker, ICD, neurostimulator, or drug pump) can comprise: using machine learning and/or artificial intelligence to identify the relationships between one or more operating parameters of the implanted medical device and lagged values (e.g. level, change rate, variation or cycle frequency over time, variation by body location, minimum value, or maximum value) of one or more biometric indicators (e.g. blood or tissue oxygen level, blood pressure, heart rate, respiration rate, body posture and/or movement, or blood glucose level).

In an example, a method for adjustment and/or control of an implanted medical device (e.g. pacemaker, ICD, neurostimulator, or drug pump) can comprise: using machine learning and/or artificial intelligence to identify the relationships between changes in one or more operating parameters of the implanted medical device and lagged changes in the values (e.g. level, change rate, variation or cycle frequency over time, variation by body location, minimum value, or maximum value) of one or more biometric indicators (e.g. blood or tissue oxygen level, blood pressure, heart rate, respiration rate, body posture and/or movement, or blood glucose level), wherein the lagged changes are measured between 1 and 10 minutes after the operating parameter changes; and subsequently adjusting the operating parameters of the implanted medical device based on measured values of these biometric indicators in light of these relationships.

In an example, a method for adjustment and/or control of an implanted medical device (e.g. pacemaker, ICD, neurostimulator, or drug pump) can comprise: using machine learning and/or artificial intelligence to identify the relationships between changes in one or more operating parameters of the implanted medical device and lagged changes in the values (e.g. level, change rate, variation or cycle frequency over time, variation by body location, minimum value, or maximum value) of one or more biometric indicators (e.g. blood or tissue oxygen level, blood pressure, heart rate, respiration rate, body posture and/or movement, or blood glucose level), wherein the lagged changes are measured between 5 and 30 minutes after the operating parameter changes; and subsequently adjusting the operating parameters of the implanted medical device based on measured values of these biometric indicators in light of these relationships.

In an example, a method for adjustment and/or control of an implanted medical device (e.g. pacemaker, ICD, neurostimulator, or drug pump) can comprise: using machine learning and/or artificial intelligence to identify the relationships between changes in one or more operating parameters of the implanted medical device and lagged changes in the values (e.g. level, change rate, variation or cycle frequency over time, variation by body location, minimum value, or maximum value) of one or more biometric indicators (e.g. blood or tissue oxygen level, blood pressure, heart rate, respiration rate, body posture and/or movement, or blood glucose level), wherein the lagged changes are measured between 25 and 100 minutes after the operating parameter changes; and subsequently adjusting the operating parameters of the implanted medical device based on measured values of these biometric indicators in light of these relationships.

In an example, a method for adjustment and/or control of an implanted medical device (e.g. pacemaker, ICD, neurostimulator, or drug pump) can comprise: using machine learning and/or artificial intelligence to identify the relationships between changes in one or more operating parameters of the implanted medical device and lagged changes in the values (e.g. level, change rate, variation or cycle frequency over time, variation by body location, minimum value, or maximum value) of one or more biometric indicators (e.g. blood or tissue oxygen level, blood pressure, heart rate, respiration rate, body posture and/or movement, or blood glucose level), wherein the lagged changes are measured between 1 and 10 minutes after the operating parameter changes.

In an example, a method for adjustment and/or control of an implanted medical device (e.g. pacemaker, ICD, neurostimulator, or drug pump) can comprise: using machine learning and/or artificial intelligence to identify the relationships between changes in one or more operating parameters of the implanted medical device and lagged changes in the values (e.g. level, change rate, variation or cycle frequency over time, variation by body location, minimum value, or maximum value) of one or more biometric indicators (e.g. blood or tissue oxygen level, blood pressure, heart rate, respiration rate, body posture and/or movement, or blood glucose level), wherein the lagged changes are measured between 5 and 30 minutes after the operating parameter changes.

In an example, a method for adjustment and/or control of an implanted medical device (e.g. pacemaker, ICD, neurostimulator, or drug pump) can comprise: using machine learning and/or artificial intelligence to identify the relationships between changes in one or more operating parameters of the implanted medical device and lagged changes in the values (e.g. level, change rate, variation or cycle frequency over time, variation by body location, minimum value, or maximum value) of one or more biometric indicators (e.g. blood or tissue oxygen level, blood pressure, heart rate, respiration rate, body posture and/or movement, or blood glucose level), wherein the lagged changes are measured between 25 and 100 minutes after the operating parameter changes.

In an example, measurement of one or more biometric indicators from one or more peripheral locations (such as a person's ears, fingers, or feet) can provide information on cardiac function and peripheral tissue health which is not available from a central location such as a person's heart or abdomen. In an example, the operating parameters of an implanted cardiac pacemaker can be automatically adjusted based on simultaneous differences in the blood or tissue oxygenation levels which are measured by external oxygen sensors on two different locations on a person's body. In an example, the operation of a plurality of implanted non-central (peripheral) blood pumps can be controlled and/or adjusted by data concerning biometric indicator levels from a plurality of wearable devices on different locations of a person's body. In an example, the ability to measure blood pressure values via sensors at one or more peripheral locations can provide more accurate measures of body-wide cardiovascular dynamics than, for example, a single central sensor.

In an example, a sensor whose data is used to adjust the operation of an implanted medical device can be an electromagnetic energy sensor which records electromagnetic brain activity. In an example, a sensor whose data is used to adjust the operation of an implanted medical device can be an EEG sensor. In an example, an EEG sensor can be an integral part of specialized smart eyewear or a modular device which attaches to the frame of conventional eyewear. In an example, analysis of a person's brainwaves can indicate whether their brain is receiving enough oxygen. Detection of a lack of oxygen can trigger additional blood flow by modifying the operating parameters of an implanted medical device. For example, blood flow can be increased by increasing the packing rate of an implanted cardiac pacemaker. In another example, blood flow can be increased by increasing the rotational speed of an impellor on an implanted blood pump.

In an example, a system or method can comprise adjusting the operating parameters of an implanted medical device (e.g. implanted pacemaker, blood pump, neurostimulator, or drug pump) implanted in a person can be based on electromagnetic activity of the person's brain. In an example, an electromagnetic brain activity sensor can be configured to collect data concerning one or more biometric indicators or conditions selected from the group consisting of: activity level, atrial fibrillation, bilirubin level, blood flow, blood or tissue oxygen (SpO2), blood pressure, bradycardia, breathing rate, calcium level, caloric intake, carbon dioxide level, carbon dioxide level, cardiopulmonary function, creatine level, eating behavior, electrolyte levels, emotional state, exercise level, eye movement, glucose level, glucose level, hormone level, hydration, hydration, hypertension, lactate level, magnesium level, oxygen saturation, pH level, potassium level, sleep or stage of sleep, speech, stress level, and troponin level.

In an example, a method can use machine learning to adjust the operation of an implanted medical device (e.g. pacemaker, ICD, neurostimulator, or drug pump) based on multivariate analysis of a person's blood or tissue oxygenation level and the person's pulse rate. In an example, a method can use machine learning to adjust the operation of an implanted medical device (e.g. pacemaker, ICD, neurostimulator, or drug pump) based on multivariate analysis of a person's blood or tissue oxygenation level and the person's heart rate. In an example, a method can use machine learning to adjust the operation of an implanted medical device (e.g. pacemaker, ICD, neurostimulator, or drug pump) based on multivariate analysis of a person's blood or tissue oxygenation level and the person's heart rate variability.

In an example, a system or method can adjust the operation of an implanted medical device (e.g. pacemaker, ICD, neurostimulator, or drug pump) based on multivariate analysis of a person's blood or tissue oxygenation level and the person's heart rate. In an example, a system or method can adjust the operation of an implanted medical device (e.g. pacemaker, ICD, neurostimulator, or drug pump) based on multivariate analysis of a person's blood or tissue oxygenation level and the person's heart rate variability. In an example, a system or method can adjust the operation of an implanted medical device (e.g. pacemaker, ICD, neurostimulator, or drug pump) based on multivariate analysis of a person's blood or tissue oxygenation level and the person's blood pressure.

In an example, a method can use machine learning to adjust the operation of an implanted medical device (e.g. pacemaker, ICD, neurostimulator, or drug pump) based on multivariate analysis of a person's blood or tissue oxygenation level and the person's blood pressure. In an example, a method can use machine learning to adjust the operation of an implanted medical device (e.g. pacemaker, ICD, neurostimulator, or drug pump) based on multivariate analysis of a person's blood pressure and the person's pulse rate. In an example, a method can use machine learning to adjust the operation of an implanted medical device (e.g. pacemaker, ICD, neurostimulator, or drug pump) based on multivariate analysis of a person's blood pressure and the person's heart rate.

In an example, a system or method can adjust the operation of an implanted medical device (e.g. pacemaker, ICD, neurostimulator, or drug pump) based on the difference between a person's blood or tissue oxygenation level measured by an implanted sensor and the person's blood or tissue oxygenation level measured by a wearable sensor. In an example, a system or method can adjust the operation of an implanted medical device (e.g. pacemaker, ICD, neurostimulator, or drug pump) based on the difference between a person's blood pressure measured by an implanted sensor and the person's blood pressure measured by a wearable sensor.

In an example, a system or method can adjust the operation of an implanted medical device (e.g. pacemaker, ICD, neurostimulator, or drug pump) based on multivariate analysis of a person's blood or tissue oxygenation level measured by an implanted sensor and the person's blood or tissue oxygenation level measured by a wearable sensor. In an example, a system or method can adjust the operation of an implanted medical device (e.g. pacemaker, ICD, neurostimulator, or drug pump) based on multivariate analysis of a person's blood pressure measured by an implanted sensor and the person's blood pressure measured by a wearable sensor.

In an example, a system for real-time adjustment of the operating parameters of a medical device (e.g. pacemaker, neurostimulator, or drug pump) implanted in a person can include a wrist band worn by that person. In an example, the wrist band can have a circumferential array of optical sensors which measure one or more biometric indicators selected from the group consisting of: blood or tissue oxygenation level; blood pressure; pulse rate; and blood flow. In an example, the wrist band can have an array of optical sensors which span at least two-thirds of the circumference of the band and measure one or more biometric indicators selected from the group consisting of: blood or tissue oxygenation level; blood pressure; pulse rate; and blood flow.

In an example, a method for adjustment and/or control of an implanted medical device (e.g. pacemaker, ICD, neurostimulator, or drug pump) can comprise: using machine learning and/or artificial intelligence to adjust one or more operating parameters of the implanted medical device based on the level, the rate of change (e.g. slope), and/or the cyclical frequency of one or more biometric indicators (e.g. blood or tissue oxygen level, blood pressure, heart rate, heart rate variability, respiration rate, body posture and/or movement, or blood glucose level) measured by a combination of implanted and wearable sensors.

In an example, a method can use machine learning to adjust the operation of an implanted medical device (e.g. pacemaker, ICD, neurostimulator, or drug pump) based on multivariate analysis of a person's blood pressure and the person's heart rate variability. In an example, a method can use machine learning to adjust the operation of an implanted medical device (e.g. pacemaker, ICD, neurostimulator, or drug pump) based on multivariate analysis of a person's blood or tissue oxygenation level measured by an implanted sensor and the person's blood or tissue oxygenation level measured by a wearable sensor. In an example, a method can use machine learning to adjust the operation of an implanted medical device (e.g. pacemaker, ICD, neurostimulator, or drug pump) based on multivariate analysis of a person's blood pressure measured by an implanted sensor and the person's blood pressure measured by a wearable sensor.

In an example, a method can use machine learning to adjust the operation of an implanted medical device (e.g. pacemaker, ICD, neurostimulator, or drug pump) based on the difference between a person's blood or tissue oxygenation level measured by an implanted sensor and the person's blood or tissue oxygenation level measured by a wearable sensor. In an example, a method can use machine learning to adjust the operation of an implanted medical device (e.g. pacemaker, ICD, neurostimulator, or drug pump) based on the difference between a person's blood pressure measured by an implanted sensor and the person's blood pressure measured by a wearable sensor. In an example, a system or method can adjust the operation of an implanted medical device (e.g. pacemaker, ICD, neurostimulator, or drug pump) based on multivariate analysis of a person's blood or tissue oxygenation level and the person's pulse rate.

In this manner, a person's cardiac functioning can be adjusted based on hydration and/or water level or changes therein. In an example, the operation of an implanted cardiac management device can be adjusted based on detected hydration and/or water level and/or changes therein. In an example, a wearable device can collect data on a biometric indicator selected from the group consisting of: oxygenation level, carbon dioxide level, lactate or lactic acid level, blood pressure, heart rate variability, pulsatile blood volume, pulsatile blood lag, hydration level, respiration rate, exhaled gas composition, body glucose level, troponin level, body motion or exercise level, and sleep status or stage. In an example, a wearable device can have a spectroscopic sensor or electromagnetic energy sensor which measures the hydration level of a person's body tissue and/or fluid. In an example, a system or method and change blood circulation by adjusting the operation of a cardiac pacemaker in response to an abnormal body hydration level.

In an example, the operation of an implanted cardiac pacemaker can be controlled and/or adjusted based on a biometric indicator selected from the group consisting of: oxygenation level, carbon dioxide level, lactate or lactic acid level, blood pressure, heart rate variability, pulsatile blood volume, pulsatile blood lag, hydration level, respiration rate, exhaled gas composition, body glucose level, troponin level, body motion or exercise level, and sleep status or stage. In an example, a system or method can adjust parameters of cardiac functioning in response to an abnormal peripherally-detected heart rate which is detected by a wearable biometric sensor (such as a wearable spectroscopic sensor). In an example, heart rate variability can be measured from multiple locations on the person's body.

This can be a significant improvement over a single central cardiac pacemaker or single central (heart-assist) blood pump which whose operation is not informed by the actual oxygen levels in a person's feet and hands. In an example, a system can increase the frequency, regularity, magnitude, and/or coordination of heart muscle contractions via an implanted cardiac management device when low oxygen levels are detected via data from a wearable electromagnetic brain activity sensor. In an example, a system can increase the magnitude of heart contractions via an implanted cardiac management device when low oxygen levels are detected in body tissue and/or fluid via data from a wearable electromagnetic brain activity sensor. In an example, a system can increase the magnitude of heart contractions via an implanted cardiac management device based on low oxygen levels detected in body tissue and/or fluid via a wearable spectroscopic sensor. More generally, a system can increase the frequency of a heart beats via an implanted cardiac management device based on low oxygen levels detected in body tissue and/or fluid via a wearable biometric sensor.

In an example, a wearable device can have an electromagnetic energy sensor which measures a person's blood pressure. In an example, a system can use machine learning and/or AI to incrementally adjust the operating parameters of a person's implanted cardiac pacemaker when the person's blood pressure is greater than 135 mmHg. In an example, abnormal blood pressure levels (or changes in those levels) can trigger changes in repeating patterns and/or transient patterns of electromagnetic brain activity. In an example, an electromagnetic brain activity sensor can be configured to collect data concerning electromagnetic brain activity which is affected by blood pressure levels (or changes in those levels). In an example, one of the biometric indicators which is measured and considered by AI for adjusting the operating parameters of an implanted medical device is blood pressure.

In an example, a method can comprise using machine learning and/or artificial intelligence to continually adjust the operating parameters of a cardiac pacemaker in order to keep the values of one or more biometric indicators within normal and/or healthy ranges. In an example, a system or method can comprise monitoring a person's blood pressure and adjusting the operating parameters of an implanted cardiac pacemaker in response to abnormal blood pressure values. In an example, automatic adjustment of cardiac functioning in response to detection of abnormal biometric indicator values can help to restore underlying biological and/or physiological processes to their proper functioning. In an example, a system or method can increase the frequency, regularity, magnitude, and/or coordination of heart muscle contractions via an implanted cardiac management device when abnormal glucose levels are detected via data from a wearable electromagnetic brain activity sensor.

In an example, a system can monitor a person's blood pressure and adjust the operating parameters of an implanted cardiac pacemaker in response to blood pressure values which are outside (e.g. lower or higher than) a normal range for that person. In an example, a system can use machine learning and/or AI to adjust right atrial pacing in an implanted cardiac pacemaker in response to changes in blood pressure. In an example, a system can use machine learning and/or AI to adjust right atrial pacing in an implanted cardiac pacemaker in response to high blood pressure. In an example, a system can use machine learning and/or AI to adjust right atrial pacing in an implanted cardiac pacemaker in response to an abnormal blood pressure level. In an example, a system can use machine learning and/or AI to incrementally adjust the operating parameters of a person's implanted cardiac pacemaker when the person's blood pressure is greater than a selected value, wherein this selected value is between 120 and 140 mmHg.

As a person's heart beats, their blood pressure varies between maximum (systolic) and minimum (diastolic) pressures. In an example, operating parameters of an implanted medical device can be automatically adjusted based on diastolic pulmonary pressure, systolic pulmonary pressure, or mean pulmonary pressure. In an example, operating parameters of an implanted medical device can be automatically adjusted based on diastolic blood pressure, systolic blood pressure, mean arterial pressure, blood pressure variability, diastolic fall time, systolic rise time. In an example, the operating parameters of a person's implanted cardiac pacemaker can be adjusted based on measured value of the person's systolic pressure.

In an example, a system or method can adjust pacing rate in an implanted cardiac pacemaker in response to changes in blood pressure. In an example, a system can adjust pacing rate in an implanted cardiac pacemaker in response to high blood pressure. In an example, a system or method can use machine learning and/or AI to adjust pacing rate in an implanted cardiac pacemaker in response to high blood pressure. In an example, a system can use machine learning and/or AI to adjust pacing rate in an implanted cardiac pacemaker in response to an abnormal blood pressure level. In an example, a system can use machine learning and/or AI to adjust pacing rate in an implanted cardiac pacemaker in response to changes in blood pressure. In an example, the pacing rate of a pacemaker can be modulated in response to a person's blood pressure (e.g. level or variation over time).

In an example, a system can adjust the operating parameters of a person's implanted cardiac pacemaker when the person's blood pressure is greater than a selected value, wherein this selected value is between 120 and 140 mmHg. In an example, a system can adjust the operating parameters of a person's implanted cardiac pacemaker when the person's blood pressure is greater than 135 mmHg. In an example, a system can adjust right atrial pacing in an implanted cardiac pacemaker in response to high blood pressure. In an example, a system can adjust right atrial pacing in an implanted cardiac pacemaker in response to an abnormal blood pressure level. In an example, a system can adjust the operation of an implanted cardiac pacemaker in response to abnormal blood pressure.

In an example, the operation of the implanted cardiac management device can be adjusted based on detected blood pressure and/or changes in blood pressure. In an example, the pacing rate of an implanted cardiac pacemaker can be adjusted in response to the person's blood pressure being out of a normal (selected) range. In an example, the value of blood pressure can be considered abnormal when it is at least Y % higher than the maximum value in a benchmark range of values, wherein Y % is selected from within a range of 5% to 25%. In an example, the value of blood pressure can be considered abnormal when it is at least X % lower than the minimum value in a benchmark range of values, wherein X % is selected from within a range of 5% to 25%.

In an example, a person's biometric indicators can be a function of the operation of an implanted medical device and other factors, machine learning and/or artificial intelligence can be used to isolate the of the implanted medical device on the indicators, and machine learning and/or artificial intelligence can be used to adjust the operating parameters of the implanted medical device to keep the biomedical parameters within normal and/or healthy ranges. In an example, a method can comprise using machine learning to adjust operating parameters of an implanted cardiac pacemaker based on biometric indicators measured by implanted and wearable sensors. In an example, a method can comprise using machine learning and/or artificial intelligence to analyze biometric indicators measured by a combination of implanted and wearable sensors and, in response, adjust the operating parameters of a vagal stimulator. In an example, machine learning and/or AI for adjusting operating parameters of an implanted medical device can employ time-series analysis.

In an example, a method or system with both a wearable PPG device and an implanted cardiac pacemaker can help to treat congestive heart failure. In an example, a peripheral feedback system with both a wearable PPG device and a implanted cardiac pacemaker can be used to help diagnosis and/or treat a condition selected from the group consisting of arrhythmia, arteriosclerosis, atherosclerosis, atherosclerotic pathology, fibrillation, autonomic dysfunction, cardio-pulmonary health, congestive heart failure, endothelial dysfunction, hypovolemia, peripheral arterial disease, poor peripheral circulation, premature ventricular contraction, stress level, and tachycardia.

In an example, a wearable device can perform photoplethysmography. Wearable photoplethysmography (PPG) devices include at least one light emitter (whose light is directed toward body tissue) and at least one light receiver (which receives this light after it has been reflected from, or transmitted through, the body tissue). In an example, a wearable photoplethysmography (PPG) device can further include one or more light barriers between a light emitter and a light receiver to block transmission of light from the emitter to the receiver except through body tissue. A wearable photoplethysmography (PPG) device and an implanted cardiac pacemaker can together comprise a system for improving cardiac function, maintaining peripheral tissue health, and/or treating a heart condition. In an example, a system for cardiac rhythm management can comprise an implanted cardiac pacemaker whose operation is automatically adjusted based on one or more biometric indicators which are measured by a wearable photoplethysmography (PPG) device.

In an example, a system can adjust a person's cardiac function based on identification of a specific type of activity based on measured whole-body posture and/or configuration. In an example, a system can increase the magnitude of a person's heart contractions via an implanted cardiac management device based on high lactate levels detected using a wearable biometric sensor. In an example, a person's cardiac functioning can be adjusted based on detected hormone levels and/or changes in hormone levels. In an example, a system can adjust the parameters of an implanted cardiac pacemaker based on a person's mean arterial pressure. In an example, a method for adjusting the operation of an implanted medical device can comprise using linear discriminant analysis to adjust operating parameters of an implanted medical device based on biometric indicators measured by implanted and wearable sensors.

In an example, a method can comprise using machine learning and/or artificial intelligence to adjust the operating parameters of an medical device implanted in a person in response to the person's heart rate (e.g. level or variability) as measured by a combination of internal biometric sensors implanted in the person and external biometric sensors worn by the person. In an example, a method can comprise using machine learning and/or artificial intelligence to adjust the operating parameters of a medical device implanted in a person in response to the person's galvanic skin response as measured by a combination of internal biometric sensors implanted in the person and external biometric sensors worn by the person. In an example, a method can comprise using non-linear programming to adjust operating parameters of an implanted cardiac pacemaker based on biometric indicators measured by implanted and wearable sensors.

In an example, biometric information from a wearable photoplethysmography (PPG) device can be used to adjust the operation of an implanted device such as a cardiac pacemaker, creating a peripheral feedback loop or (in the extreme) closed-loop system for improving cardiac function, maintaining peripheral tissue health, and/or managing a heart condition. The synergistic integration of the wearable device and the implanted cardiac rhythm management device can enable cardiac rhythm management that is superior to that provided by either component alone.

In an example, the operating parameters of a person's implanted cardiac pacemaker can be adjusted based on the difference between the person's systolic and diastolic pressures. In an example, a method can comprise using machine learning and/or artificial intelligence to adjust the operating parameters of an medical device implanted in a person in response to the person's blood pressure (e.g. systolic and/or diastolic, level, or variation) as measured by a combination of internal biometric sensors implanted in the person and external biometric sensors worn by the person.

In an example, a method for adjustment and/or control of an implanted medical device (e.g. cardiac pacemaker) can include Fourier transformation of pulse rate cycles and/or variation which is measured by a sensor worn on a person's body. In an example, a method for adjustment and/or control of an implanted medical device (e.g. cardiac pacemaker) can include Fourier transformation of blood pressure cycles and/or variation which is measured by a sensor worn on a person's body. In an example, a method for adjustment and/or control of an implanted medical device (e.g. cardiac pacemaker) can include Fourier transformation of respiration cycles and/or variation which is measured by a sensor worn on a person's body.

In an example, a system to assist human cardiovascular functioning can include: (1) an implanted cardiac pacemaker or other implanted cardiac rhythm management device, wherein the implanted device has a plurality of operating parameters including pacing rate and is implanted in a person; and (2) a wearable device worn by the person, wherein the wearable device further comprises an arcuate array of biometric sensors, wherein the implanted device and the wearable device are in wireless communication with each other, either directly or through an intermediary device, and wherein one or more of the operating parameters of the implanted cardiac pacemaker or other implanted cardiac rhythm management device are automatically adjusted based on analysis of data from the sensors.

In an example, a method can comprise using least squares estimation to adjust operating parameters of an implanted cardiac pacemaker based on biometric indicators measured by implanted and wearable sensors. In an example, a method can comprise using Fourier transformation to adjust operating parameters of an implanted cardiac pacemaker based on biometric indicators measured by implanted and wearable sensors. In an example, a method can comprise using factor analysis to adjust operating parameters of an implanted medical device based on biometric indicators measured by implanted and wearable sensors. In an example, a method can comprise using decision tree learning to adjust operating parameters of an implanted cardiac pacemaker based on biometric indicators measured by implanted and wearable sensors.

In an example, a method can comprise using artificial intelligence (AI) to adjust operating parameters of an implanted medical device based on biometric indicators measured by implanted and wearable sensors. In an example, a method can comprise using data mining to adjust operating parameters of an implanted cardiac pacemaker based on biometric indicators measured by implanted and wearable sensors. In an example, a method can comprise using correlation to adjust operating parameters of an implanted cardiac pacemaker based on biometric indicators measured by implanted and wearable sensors. In an example, a method can comprise using an artificial neural network to adjust operating parameters of an implanted medical device based on biometric indicators measured by implanted and wearable sensors.

In an example, a method can comprise using an artificial neural network (ANN) to adjust operating parameters of an implanted cardiac pacemaker based on biometric indicators measured by implanted and wearable sensors. In an example, a machine and/or artificial intelligence can learn how to continually adjust the operating parameters of a cardiac pacemaker in order to keep the values of one or more biometric indicators within normal and/or healthy ranges. In an example, a machine and/or artificial intelligence can learn how to continually adjust the operating parameters of a cardiac pacemaker in order to change the values of one or more biometric indicators in desired directions. In an example, a machine and/or artificial intelligence can learn how to continually adjust the operating parameters of a cardiac pacemaker in order to change the values of one or more biometric indicators in desired directions.

In an example, a system or method can comprise using machine learning and/or artificial intelligence to analyze biometric indicators measured by a combination of implanted and wearable sensors and, in response, adjust the operating parameters of an implantable cardiac pacemaker. In an example, a method can comprise using machine learning and/or artificial intelligence to analyze one or more biometric indicators and, in response, adjust the operating parameters of an implantable cardiac pacemaker.

In an example, artificial intelligence (AI) can be trained on historical data concerning the operating parameters of an implanted medical device and the values of biometric indicators, wherein the AI is trained to identify which set, patterns, configurations, and/or values of the operating parameters are associated with unhealthy values for the biometric indicators and which sets, patterns, configurations, and/or values of the operating parameters are associated with healthy values for the biometric indicators. In an example, machine learning and/or AI for adjusting operating parameters of an implanted medical device can employ carlavian curve analysis. In an example, a method can comprise using a Kalman filter to adjust operating parameters of an implanted cardiac pacemaker based on biometric indicators measured by implanted and wearable sensors.

In an example, machine learning and/or AI for adjusting operating parameters of an implanted medical device can employ multivariate linear regression (MLR). In an example, a method can comprise using multivariate linear regression (MLR) to adjust operating parameters of an implanted medical device based on biometric indicators measured by implanted and wearable sensors. In an example, a method can comprise using multivariate linear regression (MLR) to adjust operating parameters of an implanted cardiac pacemaker based on biometric indicators measured by implanted and wearable sensors. In an example, a method can comprise using auto-regression (AR) to adjust operating parameters of an implanted cardiac pacemaker based on biometric indicators measured by implanted and wearable sensors. In an example, machine learning and/or AI for adjusting operating parameters of an implanted medical device can employ auto-regression (AR).

In an example, a system or method can increase blood circulation by adjusting the operation of an implanted cardiac pacemaker in response to a high body lactate and/or lactic acid level. In an example, a system can change the operation of a cardiac pacemaker in response to an abnormal body glucose level. In an example, a system can change blood circulation by adjusting the operation of a cardiac pacemaker or ICD in response to troponin. In an example, a system can change blood circulation by adjusting the operation of a cardiac pacemaker in response to sleep status or stage. In an example, a system can change blood circulation by adjusting the operation of a cardiac pacemaker in response to respiration rate. In an example, a system can change blood circulation by adjusting the operation of a cardiac pacemaker in response to exhaled gas composition.

In an example, machine learning and/or AI for adjusting operating parameters of an implanted medical device can employ multivariate logit or probit. In an example, machine learning and/or AI for adjusting operating parameters of an implanted medical device can employ eigenvalue decomposition. In an example, machine learning and/or AI for adjusting operating parameters of an implanted medical device can employ a Kalman filter. In an example, machine learning and/or AI for adjusting operating parameters of an implanted medical device can employ factor analysis. In an example, machine learning and/or AI for adjusting operating parameters of an implanted medical device can employ orthogonal transformation. In an example, the operating parameters of a person's implanted cardiac pacemaker can be adjusted based on measured value of the person's diastolic pressure.

In an example, the operating parameters of an implanted cardiac pacemaker which are adjusted by a system and/or method can comprise pacing rate, maximum rate, minimum rate, pulse rate, pacing frequency, and/or stimulation frequency. In an example, machine learning and/or artificial intelligence can be used to identify the optimal set of operating parameters for an implanted medical device for a particular patient at particular time. In an example, machine learning and/or artificial intelligence can involve supervised learning in which operating parameters of an implanted device in a person during prior time periods are temporally linked to whether that person was healthy or unhealthy during those time periods. In an example, machine learning and/or artificial intelligence can be used to identify the optimal set of operating parameters for an implanted medical device for a particular patient with a particular set of biometric indicator values.

In an example, a machine and/or artificial intelligence can be trained on historical data concerning the operating parameters of a cardiac pacemaker within a person and the values of biometric indicators for that person in order to identify lagged effects of those operating parameters on those biometric indicators for that particular person. In an example, machine learning and/or artificial intelligence can consider a variety of factors other than a person's current biometric values when adjusting the operating parameters of an implanted medical device, including the person's age, the person's race, the person's gender, and the person's clinical history.

In an example, artificial intelligence (AI) can be trained on historical data on the operating parameters of an implanted medical device and the values of biometric indicators, wherein the AI is trained to identify which set, patterns, configurations, and/or values of the operating parameters are associated with moving the biometric indicators within a normal and/or healthy range. In an example, a machine and/or artificial intelligence can be trained on historical data concerning the operating parameters of a cardiac pacemaker within a person and the values of biometric indicators for that person. In an example, a machine and/or artificial intelligence can be trained on historical data concerning the operating parameters of a cardiac pacemaker within a person and the values of biometric indicators for that person in order to identify lagged effects of those operating parameters on those biometric indicators for that particular person.

In an example, a system or method for changing biometric indicators in a desired direction can comprise using machine learning and/or artificial intelligence to identify the lagged effects of operating parameters of an implanted medical device on selected biometric indicators during a first period of time; and using the identified lagged effects to adjust the operating parameters of the implanted medical device to change the biometric indicators in a desired direction and/or into a desired range of values. In an example, an implanted circulatory assistance device can be a cardiac pacemaker. In an example, an implanted circulatory assistance device can be a cardiac pacemaker or ICD. In an example, a system can adjust the parameters of an implanted cardiac pacemaker based on a person's peripheral resistance. In an example, an implanted medical device can be an artificial heart.

In an example, the operating parameters of a person's implanted cardiac pacemaker can be adjusted based on measurement of the person's blood pressure at two locations on their body. In an example, the operating parameters of a person's implanted cardiac pacemaker can be adjusted based on the difference in values of their person's blood pressure as measured at two locations on their body. In an example, the operating parameters of a person's implanted cardiac pacemaker can be adjusted based on the maximum value of their person's blood pressure as measured at two locations on their body. In an example, the operating parameters of a person's implanted cardiac pacemaker can be adjusted based on the minimum value of their person's blood pressure as measured at two locations on their body.

In an example, the operation of an implanted cardiac pacemaker can be controlled and/or adjusted by data concerning biometric indicator levels from a plurality of wearable devices on different locations of a person's body. In an example, the operation of an implanted cardiac pacemaker can be adjusted based on multivariate analysis of data concerning biometric indicator levels from a plurality of wearable devices worn at different locations on a person's body. In an example, a wearable component of a system can be worn in a manner similar to a finger ring, finger sleeve, artificial finger nail, finger nail attachment, finger tip (thimble), or glove. In an example, a wearable component of a system can be selected from the group consisting of a finger ring, finger sleeve, artificial finger nail, finger nail attachment, finger tip (thimble), and glove. In an example, the operation of an implanted cardiac pacemaker can be adjusted based on the average of biometric indicator levels, the lowest biometric indicator level, the highest biometric indicator level, and/or the range or variability of biometric indicator levels measured by a plurality of wearable devices worn at different locations on a person's body.

In an example, an implanted circulatory assistance device of a system can be a cardiac pacemaker which is in electromagnetic communication with a person's heart. In an example, a system can adjust the operation of a cardiac pacemaker in response to a high carbon dioxide level in one or more ways selected from the group consisting of increase in heart electromagnetic stimulation voltage, increase in the degree of coordination and/or timing between stimulations to different heart chambers, increase in the frequency of heart contraction stimulations, change in the locations on the heart to which electromagnetic energy is delivered, increase in the magnitude of heart contraction stimulations, increase in the regularity of heart contraction stimulations, and more precise coordination of contraction of different heart chambers.

In an example, an implanted circulatory assistance device can be am implanted cardiac pacemaker. In an example, a system can adjust the operation of a cardiac pacemaker in response to a high body motion or exercise level in one or more ways selected from the group consisting of increase in heart electromagnetic stimulation voltage, increase in the degree of coordination and/or timing between stimulations to different heart chambers, increase in the frequency of heart contraction stimulations, change in the locations on the heart to which electromagnetic energy is delivered, increase in the magnitude of heart contraction stimulations, increase in the regularity of heart contraction stimulations, delivery of an electromagnetic shock to the heart, and more precise coordination of contraction of different heart chambers.

In an example, an implanted circulatory assistance device can be am implanted cardiac pacemaker. In an example, a system can adjust the operation of a cardiac pacemaker in response to an abnormal body glucose level in one or more ways selected from the group consisting of change in heart electromagnetic stimulation voltage, change in the degree of coordination and/or timing between stimulations to different heart chambers, change in the frequency of heart contraction stimulations, change in the locations on the heart to which electromagnetic energy is delivered, change in the magnitude of heart contraction stimulations, change in the regularity of heart contraction stimulations, delivery of an electromagnetic shock to the heart, and more precise coordination of contraction of different heart chambers.

In an example, an implanted circulatory assistance device can be a cardiac pacemaker. In an example, a system can adjust the operation of a cardiac pacemaker in response to sleep status or stage in one or more ways selected from the group consisting of: change in heart electromagnetic stimulation voltage, change in the degree of coordination and/or timing between stimulations to different heart chambers, change in the frequency of heart contraction stimulations, change in the locations on the heart to which electromagnetic energy is delivered, change in the magnitude of heart contraction stimulations, change in the regularity of heart contraction stimulations, delivery of an electromagnetic shock to the heart, and more precise coordination of contraction of different heart chambers.

In an example, an implanted circulatory assistance device can be a cardiac pacemaker. In an example, a system can adjust the operation of a cardiac pacemaker in response to respiration rate in one or more ways selected from the group consisting of: change in heart electromagnetic stimulation voltage, change in the degree of coordination and/or timing between stimulations to different heart chambers, change in the frequency of heart contraction stimulations, change in the locations on the heart to which electromagnetic energy is delivered, change in the magnitude of heart contraction stimulations, change in the regularity of heart contraction stimulations, delivery of an electromagnetic shock to the heart, and more precise coordination of contraction of different heart chambers.

In an example, the operation of an implanted cardiac pacemaker can be changed by changing one or more operating parameters selected from the group consisting of frequency of heart stimulations, atrioventricular (AV) pacing interval, interventricular (IV) pacing interval, coordination between stimulations of different heart chambers, timing of stimulations of different heart chambers, location(s) of the heart to which electromagnetic energy is delivered, regularity of heart stimulations, magnitude of heart stimulations, voltage of heart stimulations, initiation of heart stimulations, and cessation of heart stimulations.

In an example, automatic adjustment of cardiac functioning in response to detection of abnormal biometric indicator values can help to restore underlying biological and/or physiological processes to their proper functioning. In an example, a method for managing cardiac functioning can adjust the operation of an implanted cardiac pacemaker in one or more ways selected from the group consisting of: increase in heart electromagnetic stimulation voltage, increase in the degree of coordination and/or timing between stimulations to different heart chambers, increase in the frequency of heart contraction stimulations, change in the locations on the heart to which electromagnetic energy is delivered, increase in the magnitude of heart contraction stimulations, increase in the regularity of heart contraction stimulations, delivery of an electromagnetic shock to the heart, and more precise coordination of contraction of different heart chambers.

In an example, a system of method can comprising adjusting the operation of an implanted cardiac management device based on detected troponin level in a person's bloodstream and/or changes in troponin level. In an example, a system or method an adjust the operation of a cardiac pacemaker or ICD in response to troponin in one or more ways selected from the group consisting of: change in heart electromagnetic stimulation voltage, change in the degree of coordination and/or timing between stimulations to different heart chambers, change in the frequency of heart contraction stimulations, change in the locations on the heart to which electromagnetic energy is delivered, change in the magnitude of heart contraction stimulations, change in the regularity of heart contraction stimulations, delivery of an electromagnetic shock to the heart, and more precise coordination of contraction of different heart chambers.

In an example, a system can adjust the operation of an implanted cardiac pacemaker in response to abnormal blood pressure in one or more ways selected from the group consisting of: change in heart electromagnetic stimulation voltage, change in the degree of coordination and/or timing between stimulations to different heart chambers, change in the frequency of heart contraction stimulations, change in the locations on the heart to which electromagnetic energy is delivered, change in the magnitude of heart contraction stimulations, change in the regularity of heart contraction stimulations, and more precise coordination of contraction of different heart chambers.

In an example, the operating parameters of an implanted cardiac pacemaker which are adjusted by a system and/or method can comprise pulse width, pacing pulse width, electrical stimulus duration, pulse duration, pacing burst length, and/or post ventricular atrial refractory period (PVARP). In an example, the operating parameters of an implanted cardiac pacemaker which are adjusted by a system and/or method can comprise stimulation location, which electrodes are selected, which heart chamber is paced, and/or which heart chamber is sensed. In an example, a system can monitor a person's systolic blood pressure and adjust the operating parameters of an implanted cardiac pacemaker in response to blood pressure values which are outside (e.g. higher than) a maximum value for that person. In an example, a method can comprise using linear discriminant analysis to adjust operating parameters of an implanted cardiac pacemaker based on biometric indicators measured by implanted and wearable sensors. In an example, a method can comprise using eigenvalue decomposition to adjust operating parameters of an implanted cardiac pacemaker based on biometric indicators measured by implanted and wearable sensors.

In an example, a machine-learning or AI method can comprise using orthogonal transformation to adjust the operating parameters of an implanted cardiac pacemaker based on biometric indicators measured by implanted and wearable sensors. In an example, the operating parameters of an implanted cardiac parameter which are adjusted can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s).

In an example, operating parameters for an implanted cardiac rhythm management device can be selected from the group consisting of: timing, rhythm, power, frequency, pattern, and/or duration of electromagnetic energy transmitted to cardiac tissue; chamber(s) or other intracardiac or extracardiac location(s) to which electromagnetic energy is transmitted; chamber(s) or other intracardiac or extracardiac location(s) from which electromagnetic energy is sensed; delay and/or offset interval(s); blanking and/or refractory period(s); lower rate and/or upper rate parameter(s); and inhibitory and/or triggering response(s). In an example, operating parameters of an implanted cardiac pacemaker which are adjusted by a system and/or method can comprise atrioventricular delay and/or interventricular delay.

In an example, a system or method can comprise using machine learning and/or artificial intelligence to analyze one or more biometric indicators and, in response, adjust the operating parameters of a cardiac rhythm device. In an example, a system or method can comprise using machine learning and/or artificial intelligence to analyze biometric indicators measured by a combination of implanted and wearable sensors and, in response, adjust the operating parameters of a cardiac rhythm device Such a system can provide a feedback loop for automatic adjustment and optimal operation of a person's cardiac pacemaker based on biometric indicators measured from one or more peripheral locations on the person's body. In an example, machine learning and/or artificial intelligence can identify person-specific causal relationships between operating parameters of a cardiac pacemaker within a person and lagged effects of those parameters on values of biometric indicators of that person.

In an example, a person's blood pressure can be measured from two locations on the person's body by two wearable devices. In an example, a rapid loss in blood pressure in a first body region relative to a second body region may indicate hemorrhaging in the first body region. In an example, a relationship between the pacing rate of a pacemaker and a person's blood pressure can be analyzed, identified, and/or quantified by a machine-learning model (e.g. AI model) and this relationship can be used for subsequent modification of the person's blood pressure by changing the pacing rate based on blood pressure level. In an example, a system can adjust pacing rate in an implanted cardiac pacemaker in response to an abnormal blood pressure level. In an example, a system can adjust right atrial pacing in an implanted cardiac pacemaker in response to changes in blood pressure.

In an example, an implanted circulatory assistance device can be an implanted cardiac pacemaker, an implanted central (heart assist) blood pump, or an implanted non-central (peripheral) blood pump. In an example, the operation of an implanted central (heart-assist) blood pump can be controlled and/or adjusted by data concerning biometric indicator levels from a plurality of wearable devices on different locations of a person's body. In an example, the operation of an implanted central (heart-assist) blood pump can be adjusted based on the average of biometric indicator levels, the lowest biometric indicator level, the highest biometric indicator level, and/or the range or variability of biometric indicator levels measured by a plurality of wearable devices worn at different locations on a person's body.

In an example, the operation of an implanted circulatory assistance device can be adjusted based on the average of biometric indicator levels, the lowest biometric indicator level, the highest biometric indicator level, and/or the range or variability of biometric indicator levels measured by a plurality of wearable devices worn at different locations on a person's body. In an example, an implanted circulatory assistance device can be a cardiac pacemaker. In an example, an implanted circulatory assistance device of a system can be a non-central (peripheral) blood pump which assists in pumping blood to a selected localized (e.g. peripheral) portion of a person's body.

In an example, a method can comprise using logit analysis to adjust operating parameters of an implanted cardiac pacemaker based on biometric indicators measured by implanted and wearable sensors. In an example, a method can comprise using factor analysis to adjust operating parameters of an implanted cardiac pacemaker based on biometric indicators measured by implanted and wearable sensors. In an example, a method can comprise using carlavian curve analysis to adjust operating parameters of an implanted cardiac pacemaker based on biometric indicators measured by implanted and wearable sensors. In an example, a method can comprise using a random forest model to adjust operating parameters of an implanted cardiac pacemaker based on biometric indicators measured by implanted and wearable sensors.

In an example, machine learning and/or AI for adjusting operating parameters of an implanted medical device can employ non-linear programming. In an example, machine learning and/or AI for adjusting operating parameters of an implanted medical device can employ variance. In an example, machine learning and/or AI for adjusting operating parameters of an implanted medical device can employ a random forest model. In an example, machine learning and/or AI for adjusting operating parameters of an implanted medical device can employ logit analysis. In an example, machine learning and/or AI for adjusting operating parameters of an implanted medical device can employ principal components analysis. In an example, the operating parameters of an implanted cardiac pacemaker which are adjusted by a system and/or method can comprise slope of acceleration, slope of deceleration, and/or deceleration rate.

In an example, the operation of a partially or fully closed-loop system for human circulatory assistance can be adjusted in real time based on analysis of data concerning one or more biometric indicators collected by one or more wearable sensors. In an example, a method can comprise using a probit model to adjust operating parameters of an implanted cardiac pacemaker based on biometric indicators measured by implanted and wearable sensors. In an example, a method can comprise using a Markov model to adjust operating parameters of an implanted cardiac pacemaker based on biometric indicators measured by implanted and wearable sensors. In an example, a method can comprise using a Bayesian network, filter, or other Bayesian analysis method to adjust operating parameters of an implanted cardiac pacemaker based on biometric indicators measured by implanted and wearable sensors.

In an example, a system can monitor a person's systolic blood pressure and diastolic blood pressure. In an example, a system can adjust the parameters of an implanted medical device based on a person's systolic blood pressure and diastolic blood pressure. In an example, a system can adjust the parameters of an implanted neurostimulator based on a person's systolic blood pressure and diastolic blood pressure. In an example, a system can adjust the parameters of an implanted cardiac pacemaker based on a person's systolic blood pressure and diastolic blood pressure.

In an example, a system or method can comprise monitoring a person's systemic vascular resistance. In an example, a system or method can adjust the parameters of an implanted medical device based on a person's systemic vascular resistance. In an example, a system can adjust the parameters of an implanted neurostimulator based on a person's systemic vascular resistance. In an example, a system can adjust the parameters of an implanted cardiac pacemaker based on a person's systemic vascular resistance.

In an example, the operating parameters of an implanted cardiac pacemaker which are adjusted by a system and/or method can comprise stimulation amplitude or magnitude, electrical stimulus amplitude, neurostimulation amplitude or voltage, pacing amplitude or voltage, pacing pulse amplitude, and/or pacing voltage. In an example, a method can comprise using variance to adjust operating parameters of an implanted cardiac pacemaker based on biometric indicators measured by implanted and wearable sensors. In an example, a method can comprise using multivariate logit or probit to adjust operating parameters of an implanted cardiac pacemaker based on biometric indicators measured by implanted and wearable sensors. In an example, a method can comprise using time-series analysis to adjust operating parameters of an implanted cardiac pacemaker based on biometric indicators measured by implanted and wearable sensors.

In an example, an implanted medical device can be a heart rhythm management device. In an example, an implanted medical device can be a cardiac pacemaker. In an example, an implanted medical device can be an ICD. In an example, an implanted medical device can be a circulatory assist device. In an example, an implanted medical device can be a mechanical circulatory support device. In an example, an implanted medical device can be a blood pump. In an example, an implanted medical device can stimulate and/or block neural transmissions. In an example, an implanted medical device can deliver spinal cord stimulation.

In an example, a method can comprise using machine learning and/or artificial intelligence to analyze one or more biometric indicators and, in response, adjust the operating parameters of an implantable cardioverter defibrillator (ICD). In an example, a method can comprise using an implanted medical device to deliver implantable cardioverter-defibrillator therapy in response to abnormal values of one or more biometric indicators. In an example, an implanted circulatory assistance device of a system can be selected from the group consisting of: cardiac rhythm management (CRM) device such as a cardiac pacemaker or implantable cardioverter-defibrillator (ICD); central (heart-assist) blood pump such as a left ventricular assist device (LVAD); and non-central (peripheral) blood pump. In an example, an implanted medical device can be a Left Ventricular Assistive Device (LVAD). In an example, an implanted blood pump can be a Left Ventricular Assist Device (LVAD). In an example, this method can comprise using machine learning and/or artificial intelligence to adjust the operating parameters of a Left Ventricular Assistive Device (LVAD).

In an example, this invention can be embodied in a system for human circulatory assistance comprising: a wearable device which is configured to be worn by a person, wherein the wearable device collects data on a biometric indicator concerning the person's body in real time; and an implanted circulatory assistance device which is configured to be implanted within the person's body, wherein the implanted circulatory assistance device assists in management of the person's cardiac rhythm and/or assists in pumping the person's blood, and wherein the operation of the implanted circulatory assistance device is controlled and/or adjusted in real time based on analysis of the biometric indicator. In an example, an implanted circulatory assistance device can be an implanted cardiac pacemaker.

In an example, a system or method can adjust the operation of an implanted medical device (e.g. pacemaker, ICD, neurostimulator, or drug pump) based on multivariate analysis of a person's blood pressure and the person's pulse rate. In an example, a system or method can adjust the operation of an implanted medical device (e.g. pacemaker, ICD, neurostimulator, or drug pump) based on multivariate analysis of a person's blood pressure and the person's heart rate. In an example, a system or method can adjust the operation of an implanted medical device (e.g. pacemaker, ICD, neurostimulator, or drug pump) based on multivariate analysis of a person's blood pressure and the person's heart rate variability.

In an example, a method can comprise receiving biometric data from a heart rate sensor. In an example, a spectroscopic sensor of a system can be configured to receive light energy which has been reflected from, or passed through, a person's blood and/or blood vessels in order to monitor for heart rate variability (HRV) and/or an irregular heartbeat. In an example, a system can increase blood circulation by adjusting the operation of a cardiac pacemaker in response to high heart rate variability. In an example, a system can increase the magnitude of a person's heart contractions via an implanted cardiac management device based on a low heart rate level in the person's body tissue and/or fluid via a wearable biometric sensor. In an example, a system can increase the magnitude of a person's heart contractions via an implanted cardiac management device based on a low heart rate level in the person's body tissue and/or fluid via a wearable spectroscopic sensor.

In an example, a method can comprise using spatial pattern recognition to adjust operating parameters of an implanted medical device based on biometric indicators measured by implanted and wearable sensors. In an example, a method can comprise using spatial pattern recognition (SPR) to adjust operating parameters of an implanted cardiac pacemaker based on biometric indicators measured by implanted and wearable sensors. In an example, machine learning and/or AI for adjusting operating parameters of an implanted medical device can employ spatial pattern recognition. In an example, the operation of an implanted cardiac function device is advantageously adjusted based on ambient noise level, spectral distribution, variability, sound pattern recognition, ambient voices, and ambient ultrasonic energy.

In an example, a spectroscopic sensor can comprise both a light emitter and a light receiver. In an example, a biometric sensor can be a light sensor (which can alternatively be called an optical sensor, optical detector, spectroscopic sensor, or spectroscopy sensor) which receives light energy which has been reflected from, or passed through, body tissue, organs, and/or fluid. In an example, a method can comprise using spectroscopic analysis (SA) to adjust operating parameters of an implanted cardiac pacemaker based on biometric indicators measured by implanted and wearable sensors. In an example, a wearable device of a system can be a spectroscopic sensor (including a light emitter and light receiver) which collects light data, wherein this data is analyzed using spectroscopic analysis in order to monitor changes in the chemical composition of body tissue and/or fluid.

In an example, a system can automatically adjust the operating parameters of an implanted medical device based on the effects of those parameters on biometric indicators which have been identified by machine learning (e.g. AI) models; measured baseline value for biometric indicators which are not optimal; and the amount and direction of adjustments required to bring the biometric indicators into normal range (and/or optimal values) from the baseline given the identified effects. In an example, a method can comprise using principal components analysis to adjust operating parameters of an implanted cardiac pacemaker based on biometric indicators measured by implanted and wearable sensors. In an example, a method can comprise using power spectrum analysis to adjust operating parameters of an implanted cardiac pacemaker based on biometric indicators measured by implanted and wearable sensors.

In an example, the operation of an implanted circulatory assistance device can be controlled and/or adjusted based on troponin level or a change thereof. In an example, the operation of the implanted circulatory assistance device can be controlled and/or adjusted based on a person's body hydration level or changes in that level. In an example, the operation of the implanted circulatory assistance device can be controlled and/or adjusted based on a person's exhaled gas composition or changes thereof. In an example, the operation of an implanted circulatory assistance device can be controlled and/or adjusted based on sleep status or stage or change thereof. In an example, the operation of an implanted circulatory assistance device can be controlled and/or adjusted based on the carbon dioxide level in a person's body tissue and/or fluid or changes in that level.

In an example, a method can comprise using machine learning and/or artificial intelligence to quantify the effects of the operating parameters of an implanted medical device on selected biometric indicators during a first period of time; and then apply these quantified effects to adjust these operating parameters to change these biometric indicators in a desired direction during a second period of time. In an example, an implanted circulatory assistance device of a system can have a first (e.g. feedback) operational mode wherein its operation is adjusted in real time based on values of a biometric indicator which are measured by a wearable device and a second (stand alone) operational mode when the wearable device is either not being worn or is not working properly.

In an example, operation of an implanted circulatory assistance device can be controlled and/or adjusted based on pulsatile blood lag or changes in pulsatile blood lag. In an example, operation of an implanted circulatory assistance device can be controlled and/or adjusted based on HRV or changes in HRV. In an example, operation of an implanted circulatory assistance device can be controlled and/or adjusted based on body glucose level or changes thereof. In an example, operation of an implanted circulatory assistance device can be controlled and/or adjusted based on lactate and/or lactic acid level in a person's body tissue and/or fluid or changes in that level. In an example, operation of an implanted circulatory assistance device can be controlled and/or adjusted based on pulsatile blood volume or variation thereof.

In an example, an implanted medical device of this system or method can comprise a pulsatile pump which produces variation in flow speed and/or pressure which is synchronized to be in phase, or out of phase, with a native cardiac pumping cycle. In an example, a blood pump can be copulsating with respect to the cardiac pumping cycle. In an example, a blood pump can be counterpulsating with respect to the cardiac pumping cycle. In an example, a plurality of extracardiac circulatory assistance devices can comprise an efficient and effective system of distributed circulatory assistance to maintain cardiac functioning and allow cardiac healing for people with CHF.

In an example, an implanted circulatory assistance device can be a central (heart-assist) blood pump which assists the heart in pumping blood or a non-central (peripheral) blood pump which assists in pumping blood through a vessel to a selected (peripheral) portion of the body. In an example, a closed loop system can increase blood circulation by adjusting the operation of a central (heart-assist) blood pump or a non-central (peripheral) blood pump. In an example, an implanted blood pump can be a magnetic flux pump. In an example, a non-central (peripheral) blood pump can be a pump with electromagnetically-driven magnetic impeller. In an example, a non-central (peripheral) blood pump can be a magnetic flux pump. In an example, an implanted circulatory assistance device of a system can be multiple non-central (peripheral) blood pumps.

In an example, an implanted circulatory assistance device of a system can be a single central (heart-assist) blood pump. In an example, an implanted circulatory assistance device can be an implanted central (heart-assist) blood pump which assists the heart in pumping blood or a non-central (peripheral) blood pump which assists in pumping blood through a vessel to a selected (peripheral) portion of the body. In an example, an implanted circulatory assistance device can be a central (heart-assist) blood pump which assists the heart in pumping blood or a non-central (peripheral) blood pump which assists in pumping blood through a vessel to a selected (peripheral) portion of the body. In an example, an implanted circulatory assistance device of a system can be an implanted central (heart-assist) blood pump which assists the heart in pumping blood. In an example, an implanted blood pump can have a low cross-sectional profile when it is not in operation and a high cross-sectional profile when it is in operation.

In an example, a closed loop system for human circulatory assistance can increase blood circulation by adjusting the operation of a non-central (peripheral) blood pump. In an example, an implanted blood pump can have a rotating impellor. In an example, an implanted blood pump can be a pump with a series of circumferentially-compressive members. In an example, an implanted blood pump can be an elastomeric pump. In an example, a non-central (peripheral) blood pump can be a pump with fluid jets which entrain native blood flow.

In an example, a method can comprise using machine learning and/or artificial intelligence to adjust the operating parameters of an implanted medical device (e.g. cardiac rhythm management device, neurostimulator, or drug pump) in a person in order to change the values of one or more selected biometric indicators for that person from unhealthy values to healthy values. In an example, a method can comprise using machine learning and/or artificial intelligence to adjust the operating parameters of an implanted medical device (e.g. cardiac rhythm management device, neurostimulator, or drug pump) in a person to: identify and quantify the effects of that device on biometric indicators; and apply knowledge of these effects to change the values of one or more selected biometric indicators for that person from unhealthy values to healthy values.

In an example, a continuous blood flow pump can contribute a sub-stream of continuous blood flow which is in addition to (and/or entrains) native pulsatile blood flow. In an example, an implanted blood pump can move blood using peristaltic motion. In an example, an implanted blood pump can increase the rate, speed, volume, and/or consistency of blood flow. In an example, a non-central (peripheral) blood pump can have an oscillating impellor. In an example, an implanted blood pump can have an oscillating impellor. In an example, a non-central (peripheral) blood pump can have a rotating impellor. In an example, the blood pump can transduce electromagnetic energy into kinetic energy.

In an example, an implanted blood pump can be an axial pump. In an example, an implanted blood pump can comprise a peristaltic pump. In an example, an implanted blood pump can be an entrainment pump. In an example, an implanted blood pump can be an electromagnetic field pump. In an example, an implanted blood pump can be an axial rotary pump. In an example, an implanted blood pump can be an axial pump. In an example, an implanted blood pump can be an Archimedes pump. In an example, an implanted blood pump can be a piston pump. In an example, an implanted blood pump can be a peristaltic pump.

In an example, an implanted blood pump can be a rotary implanted blood pump. In an example, a rotary pump can have one or more members which are rotated by a direct drive mechanical connection to an electromagnetic motor or other mechanical actuator. In an example, an implanted blood pump can move blood by means of a rotating impeller or turbine. In an example, a rotary pump can have one or more magnetic members which are rotated by magnetic interaction with an electromagnetic field. In an example, an implanted medical device can be a centrifugal blood flow pump. In an example, an implanted medical device can be an axial blood flow pump.

In an example, an implanted blood pump can be a longitudinal-membrane-wave pump. In an example, an implanted blood pump can be a hydroelastic pump. In an example, an implanted blood pump can be a diaphragm pump. In an example, an implanted blood pump can be a compressive pump. In an example, an implanted blood pump can be a centripetal (or, old school, centrifugal) pump. In an example, an implanted blood pump can be a balloon pump. In an example, an implanted blood pump can be a pump with electromagnetically-driven magnetic impeller. In an example, the blood pump of this invention can be hybrid pump which is capable of producing either a pulsatile or continuous blood flow.

In an example, an implanted medical device can be a Ventricular Assist Device (VAD). In an example, this method can comprise adjusting the flow rate of a blood pump and/or circulatory assist device. In an example, this method can comprise adjusting the maximum pressure or minimum pressure of a blood pump and/or circulatory assist device based on the values of one or more biometric indicators. In an example, this method can comprise using machine learning and/or artificial intelligence to gradually increase or decrease the flow rate of a blood pump and/or circulatory assist device based on the values of one or more biometric indicators.

In an example, a non-central (peripheral) blood pump can comprise rotating arcuate fins, vanes, or blades. In an example, an implanted blood pump can have a rotating impellor or turbine which is further comprised of one or more vanes, fins, blades, projections, winglets, airfoils, helical members, or grooves. In an example, an implanted blood pump can comprise rotating arcuate fins, vanes, or blades. In an example, an implanted blood pump can comprise a rotating impeller with multiple helical or partial-helical members.

In an example, an implanted blood pump can comprise a rotating helical or screw-shaped impeller. In an example, a non-central (peripheral) blood pump can comprise a rotating helical impellor. In an example, an implanted blood pump can comprise a rotating helical impellor.

In an example, an implanted blood pump can be a pulsatile flow pump. In an example, a blood pump can produce pulsatile blood flow and/or supplement native pulsatile blood flow. In an example, a continuous blood flow pump can have a relatively-uniform flow speed and/or pressure as long as the pump is in operation. In an example, an implanted blood pump can be a continuous flow pump. In an example, a blood pump can produce and contribute a continuous blood flow when it is in operation, but it does not have to be in operation all the time. In an example, a control unit of a system can change a blood pump from a pulsatile flow to a continuous flow.

In an example, an implanted blood pump can comprise one or more vanes, fins, blades, projections, winglets, airfoils, or helical members which rotate around an axis which is coaxial with the longitudinal axis of the blood flow lumen, with the directional vector of native blood flow, or both. In an example, an implanted blood pump can comprise one or more vanes, fins, blades, projections, winglets, airfoils, or helical members which rotate around an axis which is substantially perpendicular to the longitudinal axis of the blood flow lumen, with the directional vector of native blood flow, or both. In an example, an implanted blood pump can be a pump with fluid jets which entrain native blood flow. In an example, an implanted blood pump can move blood by sequentially inflating and deflating a series of inflatable members such as toroidal balloons along the longitudinal axis (from upstream to downstream) of an implanted blood flow lumen.

In an example, an implanted blood pump can move blood by propagating a longitudinal wave or pulse (such as a pressure wave) longitudinally (from upstream to downstream) along a flexible membrane (or other surface) which is in fluid communication with blood in an implanted blood flow lumen. In an example, an implanted blood pump can move blood by the sequential contraction (from upstream to downstream) of a series of circumferential members such as contracting bands or rings along the longitudinal axis of an implanted blood flow lumen. In an example, an implanted blood pump can comprise one or more vanes, fins, blades, projections, winglets, airfoils, or helical members which rotate around an axis which is substantially parallel with the longitudinal axis of the blood flow lumen, with the directional vector of native blood flow, or both. In an example, an implanted blood pump can move blood by sequential compression of a lumen by a longitudinally rolling member which rolls longitudinally and compressively (from upstream to downstream) along the walls of the lumen.

In an example, an implanted medical device can deliver electrical stimulation therapy to a person's vagus nerve. In an example, an implanted medical device can deliver electrical stimulation therapy within a person's body via electrodes. In an example, an implanted medical device can deliver electrical stimulation therapy to a person's brain and/or spinal cord. In an example, an implanted medical device can deliver electrical stimulation therapy to a person's heart. In an example, as part of adjusting the operating parameters of an implanted electrical medical device, a method can comprise selecting electrical stimulation pattern.

In an example, the operation of an implanted cardiac management device can be adjusted based on detection of a selected pattern of electromagnetic brain activity from data collected by the wearable component of a system. In an example, the operation of an implanted cardiac management device can be adjusted based on detection of a transient (non-repeating) pattern of electromagnetic brain activity from data collected by the wearable component of a system. In an example, one or more parameters used to identify a transient pattern of electromagnetic brain activity can be selected from the group consisting of: shape of one or more spikes; amplitude, maximum, or minimum of one or more spikes; frequency of multiple spikes; pattern covariation; pattern entropy; pattern signature; first and second order differentials; polynomial modeling; and composite sine wave modeling.

In an example, a system or method can adjust the frequency, regularity, magnitude, and/or coordination of heart muscle contractions via an implanted cardiac management device based on a person's stress level. In an example, a system can adjust the frequency, regularity, magnitude, and/or coordination of heart muscle contractions via an implanted cardiac management device based on a person's relaxation level. In an example, a system can adjust the frequency, regularity, magnitude, and/or coordination of heart muscle contractions via an implanted cardiac management device based on a person's physical activity and/or exercise level. In an example, a system can adjust the frequency, regularity, magnitude, and/or coordination of heart muscle contractions via an implanted cardiac management device based on a person's level of mental exertion or focus.

In an example, the operation of the implanted cardiac management device can be adjusted based on detection of tachycardia, bradycardia, and/or an irregular heartbeat. In an example, the operation of the implanted cardiac management device can be adjusted based on detected pH level and/or changes in pH level. In an example, the operation of the implanted cardiac management device can be adjusted based on detected lactate (and/or lactic acid) levels (or changes in those levels). In an example, the operation of the implanted cardiac management device can be adjusted based on detected hormone levels and/or changes in hormone levels. In an example, the operation of the implanted cardiac management device can be adjusted based on detected electrolyte levels and/or changes in electrolyte levels. In an example, the operation of the implanted cardiac management device can be adjusted based on detected carbon dioxide levels and/or changes in carbon dioxide levels.

In an example, this method can be implemented using a computer. In an example, this method can be implemented using a computer-readable medium. In an example, this method can be implemented via set of computer program instructions. In an example, this method can be implemented via set of computer-readable instructions. In an example, machine learning and/or artificial intelligence can occur in a data processor in a mobile computer, electronic tablet, electronic pad, mobile phone, smart phone, implanted medical device, internet-connected remote computer, communication network tower, satellite, or home control system.

In an example, a method can modify the amplitude of electrical stimulation by an implanted medical device in response to abnormal values of one or more biometric indicators. In an example, a method can modify the rate of electrical stimulation by an implanted medical device in response to abnormal values of one or more biometric indicators. In an example, a method can modify the waveform of electrical stimulation by an implanted medical device in response to abnormal values of one or more biometric indicators. In an example, the value of a biometric indicator can be considered abnormal when it is at least X % lower than the minimum value in a benchmark range of values, wherein X % is selected from within a range of 10% to 50%.

In an example, as part of adjusting the operating parameters of an implanted electrical medical device, a method can comprise selecting electrode polarities. In an example, as part of adjusting the operating parameters of an implanted electrical medical device, a method can comprise selecting electrical pulse width. In an example, as part of adjusting the operating parameters of an implanted electrical medical device, a method can comprise selecting electrode amplitude and/or voltage. In an example, as part of adjusting the operating parameters of an implanted electrical medical device, a method can comprise selecting which electrodes of used. In an example, as part of adjusting the operating parameters of an implanted electrical medical device, a method can comprise selecting electrical pulse frequency.

In an example, operating parameters of an implanted medical device can be automatically adjusted based on hemodynamic indicators. In an example, operating parameters of an implanted medical device can be automatically adjusted based on electrocardiogram pattern. In an example, operating parameters of an implanted medical device can be automatically adjusted based on blood volume, change in blood volume, or variability over time. In an example, operating parameters of a person's implanted medical device can be automatically adjusted based on the person's pulse, pulse amplitude, or Fourier pulse coefficient.

In an example, data can be transmitted wirelessly between an implanted medical device and a wearable device via far-field communication. In an example, data can be transmitted wirelessly between an implanted medical device and a wearable device via near field communication. In various examples, a system can comprise an implanted medical device which is in wireless communication with an external device selected from the group consisting of: a cell phone, an electronic tablet, electronically-functional eyewear, a home electronics portal, an implanted medical device, an internet portal, a laptop computer, a mobile computer, a mobile phone, a remote computer, a remote control unit, a smart phone, a smart utensil, a television set, and a wearable data processing hub.

In an example, a method can change (e.g. adjust) an operating parameter of an implanted medical device (within a person) in proportion to the difference between the value of a biometric indicator (of that person) and the optimal value for that indicator. In an example, a method can change (e.g. adjust) an operating parameter of an implanted medical device (within a person) in proportion to the amount by which the value of a biometric indicator (of that person) is outside a normal range for that indicator. In an example, a method can change (e.g. adjust) an operating parameter of an implanted medical device (within a person) as a linear function of the difference between the value of a biometric indicator (of that person) and the optimal value for that indicator.

In an example, a method can change (e.g. adjust) an operating parameter of an implanted medical device (within a person) by an amount which is a non-linear function of the difference between the value of a biometric indicator (of that person) and the optimal value for that indicator. In an example, a method can change (e.g. adjust) an operating parameter of an implanted medical device (within a person) by an amount which is a non-linear function of the amount by which the value of a biometric indicator (of that person) is outside a normal range for that indicator. In an example, a method can change (e.g. adjust) an operating parameter of an implanted medical device (within a person) by an amount which is a quadratic function of the difference between the value of a biometric indicator (of that person) and the optimal value for that indicator.

In an example, a method can change (e.g. adjust) an operating parameter of an implanted medical device (within a person) by an amount which is a non-linear function of the difference between the value of a biometric indicator (of that person) and the optimal value for that indicator, wherein this function has been identified by machine learning and/or artificial intelligence in a prior period. In an example, a method can change (e.g. adjust) an operating parameter of an implanted medical device (within a person) by an amount which is a quadratic function of the difference between the value of a biometric indicator (of that person) and the optimal value for that indicator, wherein this function has been identified by machine learning and/or artificial intelligence in a prior period.

In an example, a method can change (e.g. adjust) an operating parameter of an implanted medical device (within a person) by an amount which is a quadratic function of the amount by which the value of a biometric indicator (of that person) is outside a normal range for that indicator. In an example, a method can change (e.g. adjust) an operating parameter of an implanted medical device (within a person) as a linear function of the difference between the value of a biometric indicator (of that person) and the optimal value for that indicator, wherein this function has been identified by machine learning and/or artificial intelligence in a prior period. In an example, a method can change (e.g. adjust) an operating parameter of an implanted medical device (within a person) by an amount which is a linear function of the difference between the value of a biometric indicator (of that person) and the optimal value for that indicator, wherein this function has been identified by machine learning and/or artificial intelligence in a prior period.

In an example, a method can change (e.g. adjust) an operating parameter of an implanted medical device (within a person) by an amount which is a non-linear function of the amount by which the value of a biometric indicator (of that person) is outside a normal range for that indicator, wherein this function has been identified by machine learning and/or artificial intelligence in a prior period. In an example, a method can change (e.g. adjust) an operating parameter of an implanted medical device (within a person) by an amount which is a quadratic function of the amount by which the value of a biometric indicator (of that person) is outside a normal range for that indicator, wherein this function has been identified by machine learning and/or artificial intelligence in a prior period.

In an example, a method can change (e.g. adjust) an operating parameter of an implanted medical device (within a person) as a linear function of the amount by which the value of a biometric indicator (of that person) is outside a normal range for that indicator. In an example, a method can change (e.g. adjust) an operating parameter of an implanted medical device (within a person) by an amount which is a linear function of the difference between the value of a biometric indicator (of that person) and the optimal value for that indicator. In an example, a method can change (e.g. adjust) an operating parameter of an implanted medical device (within a person) by an amount which is a linear function of the amount by which the value of a biometric indicator (of that person) is outside a normal range for that indicator.

In an example, a method can change (e.g. adjust) an operating parameter of an implanted medical device (within a person) as a linear function of the amount by which the value of a biometric indicator (of that person) is outside a normal range for that indicator, wherein this function has been identified by machine learning and/or artificial intelligence in a prior period. In an example, a method can change (e.g. adjust) an operating parameter of an implanted medical device (within a person) by an amount which is a linear function of the amount by which the value of a biometric indicator (of that person) is outside a normal range for that indicator, wherein this function has been identified by machine learning and/or artificial intelligence in a prior period.

In an example, a method can comprise changing the operating parameters of an implanted medical device in an incremental, gradual, and/or iterative manner in order to not stress organs by sudden large changes and/or not to overshoot the range (and get into a dysfunctional oscillation). In an example, a method can comprise changing the operating parameters of an implanted medical device in a series of specified increments until the value of a biometric indicator affected by those changes is moved into a normal range. In an example, a method can comprise changing the operating parameters of an implanted medical device in an iterative manner, in a series of specified increments, until the value of a biometric indicator affected by those changes is moved into a normal range.

In an example, a method can comprise changing the operating parameters of an implanted medical device in an iterative manner, in a series of specified increments, until cumulative changes cause the value of the biometric indicator to be within a normal range for that indicator. In an example, a method can comprise changing the operating parameters of an implanted medical device in an iterative manner, in a series of increments of decreasing magnitude, until cumulative changes cause the value of the biometric indicator to be within a normal range for that indicator. In an example, a method can comprise changing the operating parameters of an implanted medical device in an iterative manner, in a series of increments of decreasing magnitude, until cumulative changes cause the value of the biometric indicator to be within a normal range for that indicator. In an example, operating parameters of an implanted medical device can be changed in an iterative manner, in a series of increments of decreasing magnitude, until the value of a biometric indicator affected by that parameter is moved into a normal range.

In an example, a method can comprise changing the operating parameters of an implanted medical device in an iterative manner, in a series of increments of decreasing magnitude, until the value of a biometric indicator affected by those changes is moved into a normal range. In an example, a method can comprise changing the operating parameters of an implanted medical device in an iterative manner, in a series of increments of decreasing magnitude, until the value of a biometric indicator affected by that parameter is moved into a normal range. In an example, a method can comprise changing the operating parameters of an implanted medical device in a series of specified increments until the value of a biometric indicator affected by those changes is moved into a normal range.

In an example, a method for adjusting and/or controlling the operation of an implanted medical device in a person can comprise gradually changing one or more operating parameters of the implanted medical device when a biometric parameter of that person goes outside its normal range for that person. In an example, a method for adjusting and/or controlling the operation of an implanted medical device in a person can comprise gradually (e.g. over a span of 1 to 5 minutes) changing one or more operating parameters of the implanted medical device when a biometric parameter of that person goes outside its normal range for that person. In an example, a method for adjusting and/or controlling the operation of an implanted medical device in a person can comprise gradually (e.g. over a span of 3 to 10 minutes) changing one or more operating parameters of the implanted medical device when a biometric parameter of that person goes outside its normal range for that person.

In an example, a method for adjusting and/or controlling the operation of an implanted medical device in a person can comprise gradually (e.g. over a span of 5 to 30 minutes) changing one or more operating parameters of the implanted medical device when a biometric parameter of that person goes outside its normal range for that person. In an example, a method for adjusting and/or controlling the operation of an implanted medical device in a person can comprise gradually (e.g. over a span of 10 minutes to 1 hour) changing one or more operating parameters of the implanted medical device when a biometric parameter of that person goes outside its normal range for that person.

In an example, operating parameters of an implanted medical device can be changed in an incremental manner in order to not stress organs by sudden large changes and/or not to overshoot the range (and get into a dysfunctional oscillation). In an example, operating parameters of an implanted medical device can be changed in a series of specified increments until the value of a biometric indicator affected by those changes is moved into a normal range. In an example, operating parameters of an implanted medical device can be changed in an iterative manner, in a series of specified increments, until the value of a biometric indicator affected by those changes is moved into a normal range. In an example, operating parameters of an implanted medical device can be changed in an iterative manner, in a series of increments of decreasing magnitude, until the value of a biometric indicator affected by those changes is moved into a normal range.

In an example, the value of a biometric indicator can be a lagged longitudinal function of the operating parameters of an implanted medical device. In an example, the value of a biometric indicator can be a lagged longitudinal function of the operating parameters of an implanted medical device and the operating parameters of the implanted medical device can be adjusted as a lagged longitudinal function of the value of the biometric indicator. In an example, a closed-loop system can comprise a biometric indicator whose value is a lagged function of the operating parameters of an implanted medical device, wherein the operating parameters of the implanted medical device are adjusted as lagged function of the values of the biometric indicator.

In an example, the value of a biometric indicator can be a first function of the operating parameters of an implanted medical device and the operating parameters of the implanted medical device can be adjusted as a second function of the value of the biometric indicator, wherein the two functions are optimized by a system of simultaneous equations. In an example, the value of a biometric indicator can be a first function of the operating parameters of an implanted medical device and the operating parameters of the implanted medical device can be adjusted as a second function of the value of the biometric indicator, wherein the two functions are optimized by a system of time-lagged equations.

In an example, a method for adjusting and/or controlling the operation of an implanted medical device can comprise using machine learning and/or artificial intelligence to identify the lagged effects of a change in the operating parameters of that device on one of more biometric indicators. In an example, a method for adjusting and/or controlling the operation of an implanted medical device can comprise using machine learning and/or artificial intelligence to identify the lagged effects of a change in the operating parameters of that device on one of more biometric indicators, wherein this lag in within the range of 1 to 10 minutes.

In an example, a method for adjusting and/or controlling the operation of an implanted medical device can comprise using machine learning and/or artificial intelligence to identify the lagged effects of a change in the operating parameters of that device on one of more biometric indicators. In an example, a method for adjusting and/or controlling the operation of an implanted medical device can comprise using machine learning and/or artificial intelligence to identify the lagged effects of a change in the operating parameters of that device on one of more biometric indicators, wherein this lag in within the range of 5 to 30 minutes. In an example, a method for adjusting and/or controlling the operation of an implanted medical device can comprise using machine learning and/or artificial intelligence to identify the lagged effects of a change in the operating parameters of that device on one of more biometric indicators, wherein this lag in within the range of 20 minutes to an hour.

In an example, a method for adjusting and/or controlling the operation of an implanted medical device can comprise using machine learning and/or artificial intelligence to identify the lagged effects of a change in the operating parameters of that device on one of more biometric indicators, wherein the duration of the lag is determined empirically by pattern identification. In an example, a method for adjusting and/or controlling the operation of an implanted medical device can comprise using machine learning and/or artificial intelligence to identify the lagged effects of a change in the operating parameters of that device on one of more biometric indicators, wherein the duration of the lag is determined empirically by multivariate time-series analysis.

In an example, a method for adjusting and/or controlling the operation of an implanted medical device in a person can comprise: using machine learning and/or artificial intelligence to identify the time lag between changes in one or more operating parameters of the implanted medical device and lagged changes in the values of one or more biometric indicators of that person. In an example, a method for adjusting and/or controlling the operation of an implanted medical device in a person can comprise: using machine learning and/or artificial intelligence to analyze data which includes the operating parameters of the implanted medical device over time and the values of one or more biometric indicators of that person over time in order to identify the time lag between changes in those parameters and changes in those biometric indicators (caused by changes in the parameters).

In an example, a method for moving biometric indicators in a desired direction and/or into a desired range of values can comprise receiving data concerning operating parameters during a first time period of a medical device implanted in a person; receiving data concerning the values of selected biometric indicators during the first time period for that person; using machine learning and/or artificial intelligence to identify the lagged effects of the operating parameters on the selected biometric indicators during the first time period; receiving data concerning the values of the selected biometric indicators during a second period for that person; using machine learning and/or artificial intelligence to identify whether one or more of the biometric indicators should be changed to improve the person's health; and using machine learning and/or artificial intelligence and the previously-identified effects to determine how the operating parameters of the implanted medical device should be changed to change the one or more biometric indicators to improve the person's health.

In an example, a method for moving biometric indicators in a desired direction and/or into a desired range of values can comprise receiving data concerning operating parameters of a medical device implanted in a person during a first period of time; receiving data concerning the values of selected biometric indicators for that person during the first time period; using machine learning and/or artificial intelligence to identify the lagged effects of the operating parameters on the selected biometric indicators during the first time period; receiving data concerning the values of the selected biometric indicators for that person during a second period; and using machine learning and/or artificial intelligence and the previously-identified effects to determine how the operating parameters of the implanted medical device should be changed to change the one or more biometric indicators in order to improve the person's health.

In an example, a system can comprise one or more data processing components selected from the group consisting of: data processor, data receiver, data transmitter, memory, microchip, and microprocessor. In an example, data processing can be distributed between the first and second data processors. In an example, a first data processor and/or data transmitter which is physically part of the wearable component can be in electronic communication with a second data processor and/or data receiver which is not physically part of the wearable component. In an example, an implanted cardiac rhythm management device can have a data processor. In an example, data concerning light received by a light receiver can be transmitted to the wireless data receiver and analysis of this data can occur in the data processor within the implanted cardiac rhythm management device.

In an example, a closed loop system for human circulatory assistance comprising: a first wearable device which is worn by a person on a first external location of the person's body; wherein the first wearable device further comprises a first light emitter, a first light receiver, a first data processor, and a first power source; and wherein the first wearable device collects data on a biometric indicator from the first location; a second wearable device which is worn by a person on a second external location of the person's body; wherein the second wearable device further comprises a second light emitter, a second light receiver, a second data processor, and a second power source; and wherein the second wearable device collects data on the biometric indicator from the second location; a first implanted non-central blood pump, wherein the first implanted non-central blood pump selectively increases blood flow to the first external location of the person's body based on the value of the biometric indicator at the first external location; and a second implanted non-central blood pump, wherein the second implanted non-central blood pump selectively increases blood flow to the second external location of the person's body based on the value of the biometric indicator at the second external location.

In an example, a wearable device can measure blood volume variation over time. In an example, operating parameters of an implanted medical device can be automatically adjusted based on cardiac output, change in output, or variability over time. In an example, operating parameters of an implanted medical device can be automatically adjusted based on cardiac rate, change in rate, or variability over time. In an example, operating parameters of an implanted medical device can be automatically adjusted based on blood flow, mean blood flow, maximum blood flow value, or blood flow variability over time. In an example, changes in the spectrum of light which has been reflected by body tissue and/or fluid can be analyzed to estimate the value of a biometric indicator or changes in that value over time.

In an example, the operation of an implanted cardiac function device can be advantageously adjusted based on environmental (and/or ambient) light level, spectral distribution, and/or variability. In an example, a light emitter can emit light with a first light wavelength (or wavelength range or spectral distribution) during a first time period and can emit light with a second light wavelength (or wavelength range or spectral distribution) during a second time period in response to changing environmental conditions. In an example, a light emitter can emit light energy with a first light wavelength (or wavelength range or spectral distribution) during a first time period and can emit light energy with a second light wavelength (or wavelength range or spectral distribution) during a second time period in response to changing environmental conditions.

In an example, an integrated system for managing cardiac rhythm can include both an ear ring, ear insert, or other ear-worn device and implanted device, wherein a system comprises: (a) an ear ring, ear insert, or other ear-worn device which is configured to be worn by a person, wherein the ear ring, ear insert, or other ear-worn device further comprises a light emitter which is configured to emit light toward the person's ear tissue, a light receiver which is configured to receive light from the light emitter after the light has passed through and/or been reflected from the person's ear tissue, and a wireless data transmitter; and (b) a cardiac rhythm management device which is configured to be implanted within the person, wherein the cardiac rhythm management device further comprises an electromagnetic energy emitter which is configured to deliver electromagnetic energy to the person's heart in order to manage cardiac rhythm and a wireless data receiver; (c) wherein differences between the spectral distribution of light emitted from the light emitter and the spectral distribution of light received by the light receiver are analyzed in order to measure the amount of an analyte in the person's ear tissue; and (d) wherein the operation of the cardiac rhythm management device is changed based on the amount of the analyte in the person's ear tissue.

In an example, this invention can be embodied in an integrated system for managing cardiac rhythm including both a wrist and/or arm band and an implanted device, wherein a system comprises: (a) a wrist and/or arm band which is configured to be worn by a person, wherein the wrist and/or arm band further comprises a light emitter which is configured to emit light toward the person's wrist and/or arm tissue, a light receiver which is configured to receive light from the light emitter after the light has passed through and/or been reflected from the person's wrist and/or arm tissue, and a wireless data transmitter; and (b) a cardiac rhythm management device which is configured to be implanted within the person, wherein the cardiac rhythm management device further comprises an electromagnetic energy emitter which is configured to deliver electromagnetic energy to the person's heart in order to manage cardiac rhythm and a wireless data receiver; (c) wherein differences between the spectral distribution of light emitted from the light emitter and the spectral distribution of light received by the light receiver are analyzed in order to measure the amount of an analyte in the person's wrist and/or arm tissue; and (d) wherein the operation of the cardiac rhythm management device is changed based on the amount of the analyte in the person's wrist and/or arm tissue.

In an example, an integrated system for managing cardiac rhythm can include both a wearable device and an implanted device, wherein a system comprises: (a) a wearable device which is configured to be worn by a person, wherein the wearable device further comprises a light emitter which is configured to emit light toward the person's body tissue, a light receiver which is configured to receive light from the light emitter after the light has passed through and/or been reflected from the person's body tissue, and a wireless data transmitter; and (b) a cardiac rhythm management device which is configured to be implanted within the person, wherein the cardiac rhythm management device further comprises an electromagnetic energy emitter which is configured to deliver electromagnetic energy to the person's heart in order to manage cardiac rhythm and a wireless data receiver; (c) wherein differences between the spectral distribution of light emitted from the light emitter and the spectral distribution of light received by the light receiver are analyzed in order to measure the amount of an analyte in the person's body tissue; and (d) wherein the operation of the cardiac rhythm management device is changed based on the amount of the analyte in the person's body tissue.

In an example, an integrated system for managing cardiac rhythm can include both a wearable device and an implanted device, wherein a system comprises: (a) a wearable device which is configured to be worn by a person, wherein the wearable device further comprises a light emitter which is configured to emit light toward the person's body tissue, a light receiver which is configured to receive light from the light emitter after the light has passed through and/or been reflected from the person's body tissue, and a wireless data transmitter; and (b) a cardiac rhythm management device which is configured to be implanted within the person, wherein the cardiac rhythm management device further comprises an electromagnetic energy emitter which is configured to deliver electromagnetic energy to the person's heart in order to manage cardiac rhythm and a wireless data receiver; (c) wherein differences between the spectral distribution of light emitted from the light emitter and the spectral distribution of light received by the light receiver are analyzed in order to measure the amount of an analyte in the person's body tissue; and (d) wherein the operation of the cardiac rhythm management device is changed based on the amount of the analyte in the person's body tissue.

In an example, an integrated system for managing cardiac rhythm can include both a wearable device and an implanted device, wherein a system comprises: (a) a finger ring which is configured to be worn by a person, wherein the finger ring further comprises a light emitter which is configured to emit light toward the person's finger tissue, a light receiver which is configured to receive light from the light emitter after the light has passed through and/or been reflected from the person's finger tissue, and a wireless data transmitter; and (b) a cardiac rhythm management device which is configured to be implanted within the person, wherein the cardiac rhythm management device further comprises an electromagnetic energy emitter which is configured to deliver electromagnetic energy to the person's heart in order to manage cardiac rhythm and a wireless data receiver; (c) wherein differences between the spectral distribution of light emitted from the light emitter and the spectral distribution of light received by the light receiver are analyzed in order to measure the amount of an analyte in the person's finger tissue; and (d) wherein the operation of the cardiac rhythm management device is changed based on the amount of the analyte in the person's finger tissue.

In an example, a device for sensing a biometric indicator which is part of a system to adjust an implanted medical device can comprise a plurality of light emitters (e.g. LEDs) and light receivers (e.g. photodetectors). In an example, a first light emitter can emit light at a first angle with respect to the surface of a person's body and a second light emitter can emit light at a second angle with respect to the surface of a person's body. In an example, a light receiver can receive light energy with a first light wavelength (or wavelength range or spectral distribution) during a first time period and can receive light energy with a second light wavelength (or wavelength range or spectral distribution) during a second time period. In an example, the path of light received by a light receiver can be automatically shifted by using an actuator to automatically rotate, tilt, raise, or lower a lens through which this light travels. In an example, the power and/or intensity of light emitted from a light emitter can be adjusted automatically to maintain accurate measurement of a biometric indicator value in the body even if a wearable device shifts and/or moves relative to the person's body surface.

In an example, implanted blood pumps in a plurality of internal locations can be selectively adjusted based on multivariate analysis of values of blood pressure measured from a plurality of external locations. There are many potential advantages of having a plurality of individually-controlled implanted blood pumps distributed throughout a person's body, wherein these blood pumps are adjusted (in a feedback loop) based on biometric measurements from associated external wearable devices. In an example, one or more implanted blood pumps can supplement, but not replace, native blood circulation. In an example, one or more implanted blood pumps can reduce cardiac workload without completely replacing cardiac function so that the heart may still heal and recover function—avoiding the eventual need for heart transplantation or a more-invasive full-cardiac-function replacement device.

In an example, a system or method can comprise a plurality of non-central (peripheral) blood pumps which are implanted in different peripheral blood vessels throughout a person's body. In an example, multiple non-central (peripheral) blood pumps can be configured in parallel flow or in series flow. In an example, implanted blood pumps in a plurality of internal locations can be selectively activated and/or adjusted based on multivariate analysis of values of a biometric indicator measured from a plurality of external locations. In an example, multiple non-central (peripheral) blood pumps can form a distributed network which provides extracardiac circulatory assistance. In an example, a plurality of implanted blood pumps in different locations in a person's vasculature can comprise a distributed network of circulation assisting devices for maintenance of proper blood circulation throughout different body regions.

In an example, a blood pump can be spliced into a person's vasculature in series with a natural blood vessel. In an example, a blood pump can be spliced into a person's vasculature in parallel with a natural vessel. In an example, a blood pump can be endovascularly inserted and then expanded within a peripheral blood vessel in order to provide localized circulatory assistance. In an example, an implanted blood pump can be configured to increase the flow of blood from an upstream location to a downstream location in a person's vasculature. In an example, an implanted blood pump can also improve hemodynamics. In an example, the blood pump can be defined to be in operation when it is actively transducing electromagnetic energy (such as from a battery or other electrical power source) into kinetic energy (in the form of blood flow).

In an example, the operation of an implanted blood pump in a given location can be selectively activated and/or adjusted when the value of a biometric indicator measured from that location is abnormal for at least a given length of time, wherein this length of time is selected from within a range of 10 seconds to 10 minutes. In an example, the operation of an implanted blood pump in a first location can be selectively activated and/or adjusted based on comparison of the value of a biometric indicator measured from that first location relative to the value of the biometric indicator measured from a second location. In an example, the one or more vanes, fins, blades, projections, winglets, airfoils, helical members, or grooves can transition to a different configuration when the implanted blood pump is in operation. This can allow an implanted blood pump to substantively supplement blood circulation when the mechanism is in operation, but to not substantively hinder native blood flow when the blood pump is not in operation. In an example, an implanted circulatory assistance device can be a central (heart-assist) blood pump which assists the heart in pumping blood or a non-central (peripheral) blood pump which assists in pumping blood through a vessel to a selected (peripheral) portion of the body.

In an example, the operation of a non-central (peripheral) blood pump which controls blood flow to a first body region can be adjusted to reduce blood flow to that region in response to a rapid loss in blood pressure in that body region. In an example, the operation of a non-central (peripheral) blood pump which increases blood flow to a first body region can be adjusted based on a rapid loss in blood pressure as measured by a wearable device on an external location of that first body region. In an example, the operation of an implanted blood pump in a given location can be selectively adjusted when the value of blood pressure measured from that location is abnormal for at least a given length of time, wherein this length of time is selected from within a range of 10 seconds to 10 minutes. In an example, the operation of an implanted circulatory assistance device can be controlled and/or adjusted based on a person's blood pressure or changes in their blood pressure.

In an example, a system comprising a plurality of implanted blood pumps in different locations in a person's vasculature can form a distributed network of circulation assisting devices for maintenance of proper blood circulation throughout different body regions. In an example, a closed loop system can adjust the operation of a central (heart-assist) blood pump or a non-central (peripheral) blood pump in response to abnormal pulsatile blood lag in one or more ways selected from the group consisting of: activation or deactivation of the pump to change blood flow; change in the duration of pump operation to change blood flow; change in the magnitude of pump undulation, compression, or contraction (depending on type of pump) to change blood flow; change in the speed of a pump's rotation, undulation, compression, or contraction (depending on type of pump) to change blood flow; and selective activation of a sub-set of non-central (peripheral) blood pumps to change blood flow in a selected sub-set of body locations.

In an example, the operation of an implanted non-central (peripheral) blood pump can be controlled and/or adjusted by data concerning biometric indicator levels from a plurality of wearable devices on different locations of a person's body. In an example, a closed loop system can increase blood circulation by adjusting the operation of a central (heart-assist) blood pump or a non-central (peripheral) blood pump. In an example, a closed loop system can adjust the operation of a central (heart-assist) blood pump or a non-central (peripheral) blood pump based on a person's exhaled gas composition. In an example, a closed loop system can adjust the operation of a central (heart-assist) blood pump or a non-central (peripheral) blood pump in response to an abnormal body glucose level. In an example, a closed loop system can adjust the operation of a central (heart-assist) blood pump or a non-central (peripheral) blood pump in response to sleep status or stage. In an example, a closed loop system can adjust the operation of a central (heart-assist) blood pump or a non-central (peripheral) blood pump based on a person's respiration rate.

In an example, an implanted blood flow lumen can have a longitudinal axis which is arcuate. In an example, an implanted blood flow lumen can have a longitudinal axis which follows the shape of longitudinal axis of the natural blood vessel with which the implanted blood flow lumen is in fluid communication. In an example, an implanted blood flow lumen can divide pre-implantation blood flow through a natural blood vessel from an upstream location to a downstream location into a first blood flow and a second blood flow. In an example, an implanted blood flow lumen can be selected from the group consisting of: artificial vessel segment, bioengineered vessel segment, transplanted vessel segment, artificial vessel joint, vessel branch, stent or other expandable mesh or framework, artificial lumen, manufactured catheter, manufactured tube, valve, vessel valve segment, multi-channel lumen, blood pump housing, and elastic blood chamber. In an example, a blood pump can be incorporated into an artificial blood flow lumen and/or vessel.

In an example, a method for adjusting and/or controlling the operating parameters of an implanted medical device can comprise: receiving data concerning a person's blood pressure rate from a wearable device; analyzing this data to determine if the person's blood pressure is within a normal range; and adjusting the operating parameters of an implanted medical device if the person's blood pressure is outside this normal range. In an example, a method for adjusting and/or controlling the operating parameters of an implanted medical device can comprise: receiving data concerning a person's blood pressure rate from two or more locations on the person's body; analyzing this data to evaluate differences in blood pressure between these two or more locations; and adjusting the operating parameters of an implanted medical device if these differences are abnormal.

In an example, a method for adjusting and/or controlling the operating parameters of an implanted medical device can comprise: receiving data concerning a person's blood glucose level from a wearable device; analyzing this data to determine if the person's blood glucose level is within a normal range; and adjusting the operating parameters of an implanted medical device if the person's blood glucose level is outside this normal range. In an example, a method for adjusting and/or controlling the operating parameters of an implanted medical device can comprise: receiving data concerning a person's blood glucose level from two or more locations on the person's body; analyzing this data to evaluate the magnitude of differences in blood glucose levels between these two or more locations; and adjusting the operating parameters of an implanted medical device if these differences are abnormal.

In an example, a method for adjusting and/or controlling the operating parameters of an implanted medical device can comprise: receiving data concerning a person's pulse rate from a wearable device; analyzing this data to determine if the person's pulse rate is within a normal range; and adjusting the operating parameters of an implanted medical device if the person's pulse rate is outside this normal range. In an example, a method for adjusting and/or controlling the operating parameters of an implanted medical device can comprise: receiving data concerning a person's pulse rate from two or more locations on the person's body; analyzing this data to evaluate differences in pulse rate between these two or more locations; and adjusting the operating parameters of an implanted medical device if these differences are abnormal.

In an example, machine learning and/or AI for adjusting operating parameters of an implanted medical device can employ a probit model. In an example, machine learning and/or AI for adjusting operating parameters of an implanted medical device can employ a Markov model. In an example, machine learning and/or AI for adjusting operating parameters of an implanted medical device can employ least squares estimation. In an example, machine learning and/or AI for adjusting operating parameters of an implanted medical device can employ a Bayesian network filter or other Bayesian analysis method. In an example, machine learning and/or AI for adjusting operating parameters of an implanted medical device can employ an artificial neural network.

In an example, machine learning and/or artificial intelligence for adjusting the operating parameters of an implanted medical device can use decision tree learning. In an example, machine learning and/or artificial intelligence for adjusting operating parameters of an implanted medical device can use an artificial neural network. In an example, machine learning and/or artificial intelligence for adjusting operating parameters of an implanted medical device can use Bayesian statistical methods. In an example, machine learning and/or artificial intelligence for adjusting operating parameters of an implanted medical device can use a support vector machine.

In an example, machine learning and/or artificial intelligence can use one or more of the following analytical and/or statistical methods to identify the effects of an implanted medical device on biometric indicators and manage the biometric indicators by adjusting the operating parameters of the implanted medical device: factor analysis, feature vector analysis, fisher linear discriminant, and Fourier transformation. In an example, machine learning and/or artificial intelligence can use one or more of the following analytical and/or statistical methods to identify the effects of an implanted medical device on biometric indicators and manage the biometric indicators by adjusting the operating parameters of the implanted medical device: fuzzy logic, Gaussian model, generalized auto-regressive conditional heteroscedasticity modeling, Markov model (e.g. hidden Markov model), random forest gump analysis, independent components analysis, Kalman filter, and kernel estimation.

In an example, an implanted medical device can be a neurostimulator. In an example, a method can comprise using machine learning and/or artificial intelligence to analyze one or more biometric indicators and, in response, adjust the operating parameters of a vagal stimulator. In an example, a method can comprise using machine learning and/or artificial intelligence to analyze one or more biometric indicators and, in response, adjust the operating parameters of a spinal cord stimulator. In an example, a system can adjust the parameters of an implanted neurostimulator based on a person's peripheral resistance. In an example, a system can adjust the parameters of an implanted neurostimulator based on a person's mean arterial pressure.

In an example, a method for moving biometric indicators (e.g. blood or tissue oxygenation) in a desired direction and/or into a desired range of values can comprise receiving data concerning operating parameters of an implanted cardiac rhythm device during a first time period; receiving data concerning the values of selected biometric indicators (e.g. blood or tissue oxygenation) during the first time period; using machine learning and/or artificial intelligence to identify the effects of the operating parameters on the selected biometric indicators (e.g. blood or tissue oxygenation); receiving data concerning the values of the selected biometric indicators (e.g. blood or tissue oxygenation) during a second period; using machine learning and/or artificial intelligence to identify whether one or more of the biometric indicators (e.g. blood or tissue oxygenation) should be changed; and using machine learning and/or artificial intelligence and the previously identified effects to determine how the operating parameters of the implanted cardiac rhythm device should be changed to change the one or more biometric indicators (e.g. blood or tissue oxygenation) in a desired manner.

In an example, a method can comprise using machine learning and/or artificial intelligence to quantify the effects of the operating parameters of an implanted cardiac rhythm device on selected biometric indicators (e.g. blood or tissue oxygenation) during a first period of time; and then apply these quantified effects to adjust these operating parameters to change these biometric indicators (e.g. blood or tissue oxygenation) in a desired direction during a second period of time. In an example, a method for changing biometric indicators (e.g. blood or tissue oxygenation) in a desired direction can comprise using machine learning and/or artificial intelligence to identify the lagged effects of operating parameters of an implanted cardiac rhythm device on selected biometric indicators (e.g. blood or tissue oxygenation) during a first period of time; and using the identified lagged effects to adjust the operating parameters of the implanted cardiac rhythm device to change the biometric indicators (e.g. blood or tissue oxygenation) in a desired direction and/or into a desired range of values.

In an example, a method for moving biometric indicators (e.g. blood or tissue oxygenation) in a desired direction and/or into a desired range of values can comprise receiving data concerning operating parameters during a first time period of a cardiac rhythm device implanted in a person; receiving data concerning the values of selected biometric indicators (e.g. blood or tissue oxygenation) during the first time period for that person; using machine learning and/or artificial intelligence to identify the lagged effects of the operating parameters on the selected biometric indicators (e.g. blood or tissue oxygenation) during the first time period; receiving data concerning the values of the selected biometric indicators (e.g. blood or tissue oxygenation) during a second period for that person; using machine learning and/or artificial intelligence to identify whether one or more of the biometric indicators (e.g. blood or tissue oxygenation) should be changed to improve the person's health; and using machine learning and/or artificial intelligence and the previously-identified effects to determine how the operating parameters of the implanted cardiac rhythm device should be changed to change the one or more biometric indicators (e.g. blood or tissue oxygenation) to improve the person's health.

In an example, a method for moving biometric indicators (e.g. blood or tissue oxygenation) in a desired direction and/or into a desired range of values can comprise receiving data concerning operating parameters of a cardiac rhythm device implanted in a person during a first period of time; receiving data concerning the values of selected biometric indicators (e.g. blood or tissue oxygenation) for that person during the first time period; using machine learning and/or artificial intelligence to identify the lagged effects of the operating parameters on the selected biometric indicators (e.g. blood or tissue oxygenation) during the first time period; receiving data concerning the values of the selected biometric indicators (e.g. blood or tissue oxygenation) for that person during a second period; and using machine learning and/or artificial intelligence and the previously-identified effects to determine how the operating parameters of the implanted cardiac rhythm device should be changed to change the one or more biometric indicators (e.g. blood or tissue oxygenation) in order to improve the person's health.

In an example, a method for adjusting and/or controlling the operating parameters of an implanted medical device can comprise: receiving data concerning a person's blood or tissue oxygen level from a wearable device; analyzing this data to determine if the person's blood or tissue oxygen level is within a normal range; and adjusting the operating parameters of an implanted medical device if the person's blood or tissue oxygen level is outside this normal range. In an example, a method for adjusting and/or controlling the operating parameters of an implanted medical device can comprise: receiving data concerning a person's blood or tissue oxygen level from two or more locations on the person's body; analyzing this data to evaluate differences in blood or tissue oxygen levels between these two or more locations; and adjusting the operating parameters of an implanted medical device if these differences are abnormal.

In an example, a method can analyze differences and/or variation in the values of a biometric indicator measured from different locations on a person's body. In an example, the operating parameters of a single implanted medical device can be adjusted to reduce differences and/or variation in these values. In an example, the operating parameters of a plurality of implanted medical devices can be adjusted to reduce observed differences and/or variation in these values. In an example, a method can analyze differences and/or variation in blood or tissue oxygenation levels measured from different locations on a person's body. In an example, the operating parameters of a single implanted medical device can be adjusted to reduce these differences and/or variation in oxygenation values. In an example, the operating parameters of a plurality of implanted medical devices can be adjusted to reduce differences and/or variation in these values.

In an example, a system for cardiovascular function management can include a blood pump. In an example, a closed loop system can adjust the operation of a central (heart-assist) blood pump or a non-central (peripheral) blood pump in response to body motion or exercise level. In an example, human circulatory assistance can be provided by a plurality of non-central (peripheral) blood pumps in different body regions whose operations are individually adjusted based on oxygenation levels in those respective body regions. In an example, an implanted blood pump can be a pump with a compression chamber between two one-way valves. In an example, a non-central (peripheral) blood pump can be a pump with a series of circumferentially-compressive members. In an example, a rotary implanted blood pump can have hydrodynamic or magnetic bearings.

In an example, the operation of an implanted central (heart-assist) blood pump can be controlled and/or adjusted based on a biometric indicator selected from the group consisting of: oxygenation level, carbon dioxide level, lactate or lactic acid level, blood pressure, heart rate variability, pulsatile blood volume, pulsatile blood lag, hydration level, respiration rate, exhaled gas composition, body glucose level, troponin level, body motion or exercise level, and sleep status or stage. In an example, the operation of an implanted non-central (peripheral) blood pump can be controlled and/or adjusted based on a biometric indicator selected from the group consisting of: oxygenation level, carbon dioxide level, lactate or lactic acid level, blood pressure, heart rate variability, pulsatile blood volume, pulsatile blood lag, hydration level, respiration rate, exhaled gas composition, body glucose level, troponin level, body motion or exercise level, and sleep status or stage.

In an example, a method for adjusting and/or controlling the operating parameters of an implanted medical device can include analysis of a person's electromagnetic brain activity. In an example, an electromagnetic brain activity sensor can be configured to collect data concerning electromagnetic brain activity which is affected by body tissue and/or fluid lactate (and/or lactic acid) levels (or changes in those levels). In an example, an electromagnetic brain activity sensor can be configured to collect data concerning electromagnetic brain activity which is affected by stress level. In an example, an electromagnetic brain activity sensor can be configured to collect data concerning electromagnetic brain activity which is affected by body tissue and/or fluid carbon dioxide levels (or changes in those levels).

In an example, a system can increase the frequency, regularity, magnitude, and/or coordination of heart muscle contractions via an implanted cardiac management device when high carbon dioxide levels are detected via data from a wearable electromagnetic brain activity sensor. In an example, a system can increase the frequency of a heart beats via an implanted cardiac management device when high carbon dioxide levels are detected in body tissue and/or fluid via data from a wearable electromagnetic brain activity sensor. In an example, a system can adjust parameters of cardiac functioning in response to stress level as detected by a wearable electromagnetic brain activity sensor.

In an example, a method or system can increase the frequency of a person's heart beats via an implanted cardiac management device based on high carbon dioxide levels detected in the person's body tissue and/or fluid via a wearable biometric sensor. In an example, a system can increase the magnitude of heart contractions via an implanted cardiac management device when high carbon dioxide levels are detected in body tissue and/or fluid via data from a wearable electromagnetic brain activity sensor. In an example, a method or system can increase the frequency of a person's heart beats via an implanted cardiac management device based on high lactate levels detected in the person's body tissue and/or fluid via a wearable biometric sensor. In an example, a system can increase the magnitude of heart contractions via an implanted cardiac management device when high lactate (and/or lactic acid) levels are detected in body tissue and/or fluid via data from a wearable electromagnetic brain activity sensor.

In an example, a system can adjust parameters of cardiac functioning in response to abnormal blood pressure levels detected by a wearable electromagnetic brain activity sensor. In an example, a system can decrease the frequency of a heart beats via an implanted cardiac management device when high blood pressure levels are detected in body tissue and/or fluid via data from a wearable electromagnetic brain activity sensor. In an example, a system can decrease the frequency, regularity, magnitude, and/or coordination of heart muscle contractions via an implanted cardiac management device when high blood pressure levels are detected via data from a wearable electromagnetic brain activity sensor. In an example, a system can decrease the magnitude of heart contractions via an implanted cardiac management device when high blood pressure levels are detected in body tissue and/or fluid via data from a wearable electromagnetic brain activity sensor.

In an example, a system can comprise a (partially or fully) closed-loop system for automatic adjustment of cardiac functioning via an implanted cardiac management device based on data from an electromagnetic brain activity sensor. In an example, a system or method can comprise using machine learning and/or artificial reality to adjust the operating parameters of an implanted medical in response to data from an EEG sensor. In an example, a method can comprise using machine learning and/or artificial reality to adjust the operating parameters of an implanted medical in response to brain activity data received from an EEG sensor. In an example, a wearable component of a system can be worn in a manner similar to a baseball cap, face mask, hair band, hair clip, hair comb, hair pin, hat, headband, head-encircling EEG sensor band, headphones, headset, helmet, nose plug, nose ring, respiratory mask, skull cap, tiara, or visor.

In an example, a method for adjusting and/or controlling the operating parameters of an implanted medical device can include analysis of a person's electromagnetic brain activity. In an example, the human brain can function as a biological organic sensor for monitoring biological and/or physiological processes. In an example, this pattern of electromagnetic brain activity can be a repeating waveform (or pattern) of electromagnetic brain activity. In an example, one or more electromagnetic energy sensor which collects data concerning brain activities or channels can be placed at one or more electrode placement sites selected from the group consisting of: FP1, FPz, FP2, AF7, AF5, AF3, AFz, AF4, AF6, AF8, F7, F5, F3, F1, Fz, F2, F4, F6, F8, FT7, FC5, FC3, FC1, FCz, FC2, FC4, FC6, FT8, T3/T7, C3, C4, C1, Cz, C2, C5, C6, T4/T8, TP7, CP5, CP3, CP1, CPz, CP2, CP4, CP6, TP8, T5/P7, P5, P3, P1, Pz, P2, P4, P6, T6/P8, PO7, PO5, PO3, POz, PO4, PO6, PO8, O1, Oz, and O2.

In an example, a method for adjusting and/or controlling the operating parameters of an implanted medical device can include analysis of a person's electromagnetic brain activity. In an example, this method can include adjusting these operating parameters based on identification of repeating and/or transient patterns in this brain activity. In an example, a repeating electromagnetic brain activity pattern can be decomposed into sub-patterns in different frequency bands. In an example, a repeating pattern of electromagnetic brain activity may be triggered by an abnormal value for a biological parameter or condition. In an example, a transient pattern of electromagnetic brain activity may be triggered by an abnormal value for a biological parameter or condition. In an example, a transient pattern of electromagnetic brain activity can be triggered by an internal biological and/or physiological event. In an example, a transient pattern of electromagnetic brain activity can be triggered by an external sensory stimulus and/or environmental event.

In an example, a method for adjusting and/or controlling the operating parameters of an implanted medical device can include analysis of a person's electromagnetic brain activity. In an example, an electromagnetic energy sensor that collects data concerning brain activity can be a dry electrode. In an example, an electromagnetic energy sensor that collects data concerning brain activity can be a capacitive sensor. In an example, an electromagnetic energy sensor can collect data concerning changes in transmission of electromagnetic energy from the emitter to the receiver due to changes in electromagnetic brain activity. In an example, an electromagnetic energy sensor which collects data concerning brain activity can measure voltage fluctuations between a first electrode and a second (reference) electrode due to electromagnetic brain activity.

In an example, a method for adjusting and/or controlling the operating parameters of an implanted medical device can include analysis of a person's electromagnetic brain activity. In an example, an electromagnetic brain activity sensor can be configured to collect data concerning electromagnetic brain activity which is associated with sleep and/or different stages of sleep. In an example, an electromagnetic brain activity sensor can be configured to collect data concerning electromagnetic brain activity which is affected by relaxation level. In an example, an electromagnetic brain activity sensor can be configured to collect data concerning electromagnetic brain activity which is affected by level of mental exertion or focus. In an example, an electromagnetic brain activity sensor can be configured to collect data concerning electromagnetic brain activity which is affected by body tissue and/or fluid glucose levels (or changes in those levels).

In an example, a method can comprise using machine learning and/or artificial intelligence to adjust the operating parameters of a medical device implanted in a person in response to the person's electroencephalogram (EEG) pattern as measured by a combination of internal biometric sensors implanted in the person and external biometric sensors worn by the person. In an example, a repeating electromagnetic brain activity pattern can be modeled as a composite of multiple sine waves. A transient pattern of electromagnetic brain activity can be a sequence of spikes or waves which do not repeat in a regular or ongoing manner. Ongoing brain waveforms classified as Alpha waves can be within a frequency band selected from the group consisting of: 7-13 Hz, 7-14 Hz, 8-12 Hz, 8-13 Hz, 7-11 Hz, 8-10 Hz, and 8-10 Hz. Ongoing brain waveforms classified as Beta waves can be within a frequency band selected from the group consisting of: 11-30 Hz, 12-30 Hz, 13-18 Hz, 13-22 Hz, 13-26 Hz, 13-26 Hz, 13-30 Hz, 13-32 Hz, 14-24 Hz, 14-30 Hz, and 14-40 Hz. Ongoing brain waveforms classified as Delta waves can be within a frequency band selected from the group consisting of: 0.5-3.5 Hz, 0.5-4 Hz, 1-3 Hz, 1-4 Hz, and 2-4 Hz. Ongoing brain waveforms classified as Gamma waves can be within a frequency band selected from the group consisting of: group consisting of: 30-100 Hz, 35-100 Hz, 40-100 Hz, and greater than 30 Hz. Ongoing brain waveforms classified as Theta waves can be within a frequency band selected from the group consisting of: from the group consisting of: 3.5-7 Hz, 3-7 Hz, 4-7 Hz, 4-7.5 Hz, 4-8 Hz, and 5-7 Hz.

In an example, a method and system for adjusting and/or controlling an implanted medical device can include a human-to-computer interface which requires action by the person in whom the device is implanted. In an example, a control method or system can include active input from a human-to-computer interface as well as passive input from implanted and/or wearable sensors which automatically collect biometric information concerning the person. In an example, a human-to-computer interface can comprise a touch screen on a smart watch or wrist band worn by the person. In an example, one or more human-computer-interface components can be selected from the group consisting of: touch screen, gesture recognition interface, speech and/or voice recognition interface, button and/or keypad, dial and/or knob, brainwave-based HCI, and motion sensor.

In an example, an implanted medical device can deliver deep brain stimulation. In an example, a method can comprise using machine learning and/or artificial intelligence to analyze biometric indicators measured by a combination of implanted and wearable sensors and, in response, adjust the operating parameters of a deep brain stimulation (DBS) device. In an example, a method can comprise using machine learning and/or artificial intelligence to analyze one or more biometric indicators and, in response, adjust the operating parameters of a deep brain stimulation (DBS) device.

In an example, a method can comprise receiving biometric data from a microphone, heart sound sensor, and/or ultrasound sensor. In an example, a system or method can adjust the parameters of an implanted medical device based on a person's peripheral resistance. In an example, a system or method can adjust the parameters of an implanted medical device based on a person's mean arterial pressure. In an example, a system or method can adjust the parameters of an implanted medical device based on variation in the lag time between central cardiac pulsation and peripheral blood pulsation. In an example, the operation of this device can also be automatically adjusted, modified, and/or controlled based on data from one or more environmental sensors.

In an example, a method or system can comprise a (partially or fully) closed-loop system for automatic adjustment of cardiac functioning via an implanted cardiac management device based on data from one or more wearable biometric sensors. In an example, a system can increase (or decrease) the frequency of a person's heart beats and/or the magnitude of a person's heart contractions in response to an increase (or decrease) in the person's skin moisture level as detected by a wearable biometric sensor. In an example, a system can increase (or decrease) the frequency of a person's heart beats and/or the magnitude of a person's heart contractions in response to an increase (or decrease) in the person's tissue impedance level as detected by a wearable biometric sensor.

In an example, a method can comprise using machine learning and/or artificial intelligence to analyze biometric indicators measured by a combination of implanted and wearable sensors and, in response, adjust the operating parameters of a spinal cord stimulator. In an example, a method can comprise using machine learning and/or artificial intelligence to analyze biometric indicators measured by a combination of implanted and wearable sensors and, in response, adjust the operating parameters of a cardiac stimulation device. In an example, a method can comprise using machine learning and/or artificial intelligence to analyze biometric indicators measured by a combination of implanted and wearable sensors and, in response, adjust the operating parameters of a heart failure device.

In an example, a closed-loop system for adjusting operating parameters of an implanted medical device can include a temperature sensor, skin temperature sensor, and/or ambient temperature sensor. In an example, the operation of the implanted cardiac management device can be adjusted based on detected body temperature and/or changes in body temperature. In an example, a system can include one or more wearable sensors selected from the group consisting of temperature sensor, skin temperature sensor, and ambient temperature sensor. In an example, a method can comprise receiving biometric data from a temperature sensor, skin temperature sensor, and/or ambient temperature sensor. In an example, a person's cardiac functioning can be adjusted based on detected body temperature and/or changes in body temperature.

In an example, a method can comprise using machine learning and/or artificial intelligence to analyze one or more biometric indicators and, in response, adjust the operating parameters of a cardiac resynchronization therapy (CRT) device. In an example, a method can comprise using an implanted medical device to deliver cardiac resynchronization therapy in response to abnormal values of one or more biometric indicators. In an example, a method can comprise using machine learning and/or artificial intelligence to analyze biometric indicators measured by a combination of implanted and wearable sensors and, in response, adjust the operating parameters of a cardiac resynchronization therapy (CRT) device.

In an example, a system can include one or more environmental sensors. In an example, the operation of an implanted cardiac function device can be advantageously adjusted based on environmental (and/or ambient) conditions such as temperature, humidity, altitude, and barometric pressure. In an example, a system can comprise an array of light emitters wherein different emitters in this array emit light based on data from one or more environmental sensors in response to different environmental parameters or conditions. In an example, different receivers in this array can receive light during different environmental conditions. In an example, a first light receiver can receive light during a first environmental condition and a second light receiver can receive light during a second environmental condition.

In an example, a biometric indicator which is measured and managed by a system or method can be a person's respiration rate. In an example, a method can comprise using machine learning and/or artificial intelligence to adjust the operating parameters of a medical device implanted in a person in response to the person's respiratory rate as measured by a combination of internal biometric sensors implanted in the person and external biometric sensors worn by the person. In an example, a person's respiration rate can be measured by measuring electromagnetic energy from (and/or passing electromagnetic energy through) body tissue. In an example, a system can adjust a person's cardiac function based on their respiration rate. In an example, the operation of the implanted circulatory assistance device can be controlled and/or adjusted based on respiration rate or changes thereof.

In an example, a system can comprise one or more motion sensors. In an example, the operation of an implanted circulatory assistance device can be controlled and/or adjusted based on body motion or exercise level or changes thereof. In an example, body motion and/or configuration sensors can be used to identify whether the person is engaged in one or more selected types of physical activities. In an example, a wearable device can have a motion sensor or EEG sensor which measures sleep status or stage. In an example, a closed-loop system for adjusting operating parameters of an implanted medical device can include a motion sensor, accelerometer, patient movement sensor, and/or gyroscope.

In an example, data concerning one or more biometric indicators can be obtained from a combination of internal (e.g. implanted) sensors and external (e.g. wearable) sensors on an elastic band which is worn by the person. In an example, data concerning one or more biometric indicators can be obtained from a combination of internal (e.g. implanted) sensors and external (e.g. wearable) sensors on an earbud or hearing aid which is worn by the person. In an example, data concerning one or more biometric indicators can be obtained from a combination of internal (e.g. implanted) sensors and external (e.g. wearable) sensors on an adhesive patch which is worn by the person. In an example, data concerning one or more biometric indicators can be obtained from a combination of internal (e.g. implanted) sensors and external (e.g. wearable) sensors on a wrist band and/or arm band which is worn by the person.

In an example, a system or method can increase the magnitude of heart contractions via an implanted cardiac management device when high lactate (and/or lactic acid) levels are detected via data from an electromagnetic muscle activity sensor. In an example, a system can increase the magnitude of heart contractions via an implanted cardiac management device when a high whole-body activity level is detected via data from an electromagnetic muscle activity sensor. In an example, a system can increase the frequency, regularity, magnitude, and/or coordination of heart muscle contractions via an implanted cardiac management device when high lactate (and/or lactic acid) levels are detected via data from an electromagnetic muscle activity sensor. In an example, a system can increase the frequency, regularity, magnitude, and/or coordination of heart muscle contractions via an implanted cardiac management device when a high whole-body activity level is detected via data from an electromagnetic muscle activity sensor.

In an example, a system or method can increase the frequency of a heart beats via an implanted cardiac management device when high lactate (and/or lactic acid) levels are detected via data from an electromagnetic muscle activity sensor. In an example, a system can increase the frequency of a heart beats via an implanted cardiac management device when a high whole-body activity level is detected via data from an electromagnetic muscle activity sensor. In an example, a system can decrease the magnitude of heart contractions via an implanted cardiac management device when a low whole-body activity level is detected via data from an electromagnetic muscle activity sensor. In an example, a system can decrease the frequency of a heart beats via an implanted cardiac management device when a low whole-body activity level is detected via data from an electromagnetic muscle activity sensor.

In an example, a system can increase the frequency, regularity, magnitude, and/or coordination of a person's heart muscle contractions via an implanted cardiac management device based on a low heart rate level detected in the person's body tissue and/or fluid via a wearable light energy and/or electromagnetic energy sensor. In an example, a system can increase the frequency, regularity, magnitude, and/or coordination of a person's heart muscle contractions via an implanted cardiac management device based on high carbon dioxide levels detected in the person's body tissue and/or fluid via a wearable light energy and/or electromagnetic energy sensor.

In an example, a system can increase (or decrease) the magnitude of a person's heart contractions via an implanted cardiac management device based on low (or high) glucose levels detected in the person's body tissue and/or fluid via a wearable biometric sensor. In an example, a system can increase (or decrease) the frequency, regularity, magnitude, and/or coordination of a person's heart muscle contractions via an implanted cardiac management device based on low (or high) glucose levels detected in the person's body tissue and/or fluid via a wearable light energy and/or electromagnetic energy sensor. In an example, a system can increase (or decrease) the frequency of a person's heart beats via an implanted cardiac management device based on low (or high) glucose levels detected in the person's body tissue and/or fluid via a wearable biometric sensor.

In an example, a system or method can increase (or decrease) the frequency, regularity, magnitude, and/or coordination of a person's heart muscle contractions via an implanted cardiac management device based on a low (or high) blood pressure level detected in the person's body tissue and/or fluid via a wearable light energy and/or electromagnetic energy sensor. In an example, a system can increase (or decrease) the magnitude of a person's heart contractions via an implanted cardiac management device based on a low (or high) blood pressure level in the person's body tissue and/or fluid via a wearable biometric sensor. In this manner, the person's cardiac functioning can be adjusted based on detected blood pressure and/or changes in blood pressure. These changed patterns are detected by analysis of data from one or more wearable electromagnetic energy sensors, which triggers adjustment of cardiac function (via the implanted cardiac management device) which, in turn, restores normal blood pressure levels.

In an example, a system can comprise one or more electromagnetic sensors which measure the conductivity, resistance, impedance, capacitance, and/or permittivity of body tissue and/or fluid. In an example, a method can comprise receiving biometric data from an electromagnetic energy sensor, bioimpedance sensor, skin and/or tissue impedance sensor, resistance sensor, galvanic skin response (GSR) sensor, ECG sensor, EEG sensor, and/or electrochemical sensor. In an example, operating parameters of an implanted medical device can be automatically adjusted based on intracardiac impedance or intrathoracic impedance. In an example, a method can comprise using machine learning and/or artificial intelligence to adjust the operating parameters of an medical device implanted in a person in response to the person's impedance (e.g. tissue, skin) as measured by a combination of internal biometric sensors implanted in the person and external biometric sensors worn by the person.

In an example, a closed-loop system for adjusting operating parameters of an implanted medical device can include an electromagnetic energy sensor, bioimpedance sensor, skin and/or tissue impedance sensor, resistance sensor, galvanic skin response (GSR) sensor, ECG sensor, EEG sensor, and/or electrochemical sensor. In an example, a closed-loop system for adjusting operating parameters of an implanted medical device can include a microphone, heart sound sensor, and/or ultrasound sensor. When the biometric indicator is outside (lower or higher than) the normal range, then the operating parameters of the implanted medical device are changed in order to bring the indicator within range.

In an example, a closed-loop system for adjusting operating parameters of an implanted medical device can include a humidity and/or moisture sensor. In an example, a closed-loop system for adjusting operating parameters of an implanted medical device can include a chemical sensor. In an example, a closed-loop system for adjusting operating parameters of an implanted medical device can include a camera. In an example, a closed-loop system for adjusting operating parameters of an implanted medical device can include a blood glucose sensor. For example, detection of high lactate (and/or lactic acid) levels by a wearable electromagnetic energy sensor can trigger increased blood flow which, in turn, can help to lower lactate (and/or lactic acid) levels. For example, detection of a high carbon dioxide levels by a wearable electromagnetic energy sensor can trigger increased blood flow which, in turn, can help to lower carbon dioxide levels.

In an example, an implanted medical device can electronically paired (e.g. through wireless electronic communication) with a wearable device, wherein operating parameters of the implanted medical device are adjusted and/or controlled based on the values of a biometric indicator measured by a sensor on the wearable device. In an example, a first implanted medical device implanted in a person can electronically paired (e.g. through wireless electronic communication) with a first wearable device worn by the person, wherein operating parameters of the first implanted medical device are adjusted and/or controlled based on the values of a biometric indicator measured by a sensor on the first wearable device; and a second implanted medical device implanted in the person can electronically paired (e.g. through wireless electronic communication) with a second wearable device worn by the person, wherein operating parameters of the second implanted medical device are adjusted and/or controlled based on the values of a biometric indicator measured by a sensor on the second wearable device.

In an example, a method can comprise using machine learning and/or artificial intelligence to adjust the operating parameters of an medical device implanted in a person in response to the person's body motion level (e.g. as measured an inertial motion unit) as measured by a combination of internal biometric sensors implanted in the person and external biometric sensors worn by the person. In an example, a method can comprise using machine learning and/or artificial intelligence to adjust the operating parameters of an medical device implanted in a person in response to the person's electrocardiogram (ECG) pattern as measured by a combination of internal biometric sensors implanted in the person and external biometric sensors worn by the person.

In an example, a method for adjusting and/or controlling the operation of an implanted medical device can be based on data concerning a plurality of biometric indicators received from a combination of different types of sensors including motion sensor, optical sensors, and electromagnetic energy sensors. In an example, a method for adjusting and/or controlling the operation of an implanted medical device can be based on data concerning a plurality of biometric indicators received from a combination of different types of wearable sensors including motion sensor, optical sensors, and electromagnetic energy sensors. In an example, a method for adjusting and/or controlling the operation of an implanted medical device can be based on data concerning a plurality of biometric indicators received from a combination of different types of implanted and wearable sensors including motion sensor, optical sensors, and electromagnetic energy sensors.

In an example, data concerning one or more biometric indicators can be obtained from sensors on a pendant which is worn by the person. In an example, a wearable component of a system can be a shirt, undershirt, bra, belt, collar, jacket, necklace, chest strap, waist band, waist strap, or compression belt. In an example, a biometric sensor of a system can be integrated into a shirt, undershirt, bra, belt, collar, jacket, necklace, chest strap, waist band, waist strap, or compression belt. In an example, data concerning one or more biometric indicators can be obtained from a combination of internal (e.g. implanted) sensors and external (e.g. wearable) sensors on a pendant which is worn by the person. In an example, a wearable device of a system can be selected from the group consisting of: necklace or pendant, hair comb or band, earpiece, bracelet or bangle, earring, skull cap, Augmented Reality (AR) eyewear, electronically-functional eyewear, wrist strap, buckle, sleeve, face mask or goggles, ear bud, and finger nail attachment.

In an example, a method for modifying the operation of an implanted medical device can include analysis of data from one or more external sensors which are part of one or more wearable devices. In an example, a wearable device can be a wrist band, smart watch, or bracelet. In an example, a wearable device can be an ear ring, earphone, or ear bud. In an example, a wearable device can be a finger ring. In an example, a wearable device can be smart eyeglasses (e.g. augmented reality eyewear). In an example, this method can include analysis of data from sensors which are embodied in smart clothing (e.g. shirt, sock, shoe, pair of pants, underpants, or bra).

In an example, a system can trigger an implanted medical device to deliver a therapy program (sequence of parameter adjustments) in response to biometric values (measured by internal and external sensors, out of normal range, etc.). In an example, adjustments can be iterative and in progressively-smaller amounts to avoid overshooting. In an example, as the absolute value of the difference becomes smaller, the size of the adjustment can become smaller. In an example, a system can titrate therapy from an implanted medical device based on lagged effects measured by a combination of internal and external sensors. In an example, a system can titrate therapy from an implanted medical device based on feedback from a combination of internal and external sensors. In an example, a system can change the parameters of therapy provided to a person by an implanted medical device in response to changes in the person's symptoms and/or progression of a condition. In an example, a method can adjust the therapy provided to a person by an implanted medical device in response to changes in the person's symptoms and/or progression of a condition.

In an example, a method can comprise using machine learning and/or artificial intelligence to identify relationships between the operating parameters of a medical device implanted in a person's body and biometric indicators measured by sensors worn on the person's body. In an example, a method can comprise using machine learning and/or artificial intelligence to identify relationships between the operating parameters of a medical device implanted in a person's body and the values of biometric indicators measured by sensors worn on the person's body based on historical data concerning those operating parameters and those values. In an example, a method can comprise using machine learning and/or artificial intelligence to identify time-lagged relationships between the operating parameters of a medical device implanted in a person's body and the values of biometric indicators measured by sensors worn on the person's body based on historical data concerning those operating parameters during first time periods and those values during second (lagged) time periods.

In an example, a method can comprise using machine learning and/or artificial intelligence to identify time-lagged relationships for a specific person between the operating parameters of a medical device implanted in that person's body and the values of biometric indicators measured by sensors worn on that person's body. In an example, a method can comprise using machine learning and/or artificial intelligence to identify person-specific time-lagged relationships between the operating parameters of a medical device implanted in a person's body and the values of biometric indicators measured by sensors worn on that person's body.

In an example, a method can comprise using linear regression based machine learning to identify the relationships between the operating parameters of an implanted medical device and the values of biometric indicators measured by (implanted and wearable) sensors. In an example, a method can comprise using machine learning to identify the relationship between operating parameters of an implanted medical device and values of a biometric indicator during a first time and adjust the operating parameters of the implanted medical device based on the value of the biometric indicator at a second time. In an example, a method can comprise using machine learning to identify the relationship between operating parameters of an implanted medical device and lagged values of a biometric indicator during a first time and adjust the operating parameters of the implanted medical device based on the value of the biometric indicator at a second time.

In an example, a method for moving biometric indicators in a desired direction and/or into a desired range of values can comprise receiving data concerning operating parameters of a medical device implanted in a person during a first period of time; receiving data concerning the values of one or more biometric indicators for that person from a combination of implanted sensors and wearable sensors during the first time period; using machine learning and/or artificial intelligence to identify the lagged effects of the operating parameters on the biometric indicators during the first time period; receiving data concerning the values of the biometric indicators for that person from a combination of implanted sensors and wearable sensors during a second period; and using machine learning and/or artificial intelligence to evaluate the values of the biometric indicators during the second period and the previously-identified effects in order to determine how the operating parameters of the implanted medical device should be changed in order to change the one or more biometric indicators to improve the person's health.

In an example, a method for moving biometric indicators in a desired direction and/or into a desired range of values can comprise receiving data concerning operating parameters of a medical device implanted in a person during a first period of time; receiving data concerning the values of one or more biometric indicators for that person from a combination of implanted sensors and wearable sensors during the first time period; using machine learning and/or artificial intelligence to identify the lagged effects of the operating parameters on the biometric indicators during the first time period; receiving data concerning the values of the biometric indicators for that person from a combination of implanted sensors and wearable sensors during a second period; evaluating whether the values are within a normal and/or healthy range; and if the values are not within the normal and/or healthy range, using machine learning and/or artificial intelligence to determine how the operating parameters of the implanted medical device should be changed in order to move the values into the normal and/or healthy range.

In an example, a wearable component of a system can include an electromagnetic energy sensor which measures electromagnetic brain activity. In an example, a wearable component of a system can be selected from the group consisting of: baseball cap, face mask, hair band, hair clip, hair comb, hair pin, hat, headband, head-encircling EEG sensor band, headphones, headset, helmet, nose plug, nose ring, respiratory mask, skull cap, tiara, and visor. In an example, the operation of an implanted cardiac management device can be adjusted based on detection of a selected pattern of electromagnetic brain activity from a particular electromagnetic energy sensor location, a particular channel, and/or particular montage of channels.

In an example, a system can increase (or decrease) the frequency of a person's heart beats and/or the magnitude of a person's heart contractions in response to a change in the person's whole-body posture and/or configuration as detected by one or more wearable biometric sensors. Automatic adjustment of cardiac functioning and/or blood circulation in real time in response to abnormal biometric values measured by wearable devices can help to maintain healthy biological processes and prevent tissue degradation. In an example, adjustments to the operating parameters of an implanted medical device can be done in an iterative manner until the values of selected biometric indicators are within healthy ranges. In an example, a closed loop system for human circulatory assistance can comprise a plurality of wearable devices which are worn on different locations of a person's body so as to measure values of a biometric indicator from different locations on the person's body.

In an example, detection of a high blood pressure levels by a wearable electromagnetic energy sensor can trigger an implanted to decrease blood flow which, in turn, can help to lower blood pressure levels. For example, detection of high blood pressure in peripheral tissue (or organs) by a wearable biometric sensor can trigger an implanted device to decrease blood flow, which in turn can help to restore proper blood pressure in that tissue (or organs). In an example, blood pressure can be measured from multiple locations on a person's body. In an example, a blood pressure sensor can be incorporated into the textile of a smart garment—a clothing-integrated blood pressure cuff. In an example, a closed-loop system for adjusting operating parameters of an implanted medical device can include a pressure sensor, blood pressure sensor, and/or blood pressure cuff.

In an example, the system can comprise a closed-loop control mechanism wherein the operating parameters of an implanted medical device are adjusted based on a plurality of biometric indicators measured by a combination of internal (implanted) and external (worn) sensors. In an example, the operation of an implanted circulatory assistance device can be controlled and/or adjusted by data concerning biometric indicator levels from a plurality of wearable devices on different locations of a person's body. In an example, data concerning one or more biometric indicators can be obtained from a combination of internal (e.g. implanted) sensors and external (e.g. wearable) sensors on eyewear (e.g. eyeglasses) which is worn by the person.

In an example, the operation of an implanted circulatory assistance device can be adjusted based on multivariate analysis of data concerning biometric indicator levels from a plurality of wearable devices worn at different locations on a person's body. In an example data from wearable sensors can be analyzed using an analytical method selected from the group consisting of: Least Squares Estimation, Linear Regression, Linear Transform, Logit model, machine learning (ML), Markov model, Maximum Entropy modeling, Maximum Likelihood, Mean Power, Multi-Band Covariance analysis, Multi-Channel Covariance analysis, and Multivariate Linear Regression.

In an example, the operation of an implanted blood pump in a first location can be selectively adjusted based on comparison of the value of blood pressure measured from that first location relative to the value of blood pressure measured from a second location. In an example, an implanted blood pump in a first location can be selectively activated and/or adjusted when the value of a biometric indicator measured from that first location is at least X % lower or Y % higher than the value of the biometric indicator measured from a second location. In an example, an electromagnetic energy sensor can comprise an electromagnetic energy emitter at a first location relative to body tissue and an electromagnetic energy receiver at a second location relative to body tissue, wherein the electromagnetic energy receiver receives energy which has been transmitted from the electromagnetic energy emitter through body tissue.

In an example, the biometric indicator which is measured and managed by a system can be blood pressure. In an example, the biometric sensors can be blood pressure sensors. In an example, multivariate analysis of blood pressure, blood volume variation, and other biometric indicators from sensors on wearable devices at different external locations of a person's body can be analyzed in real time to detect rapid blood loss from a selected body region. In an example, an implanted blood pump in a first location can be selectively adjusted when the value of blood pressure measured from that first location is at least X % lower or Y % higher than the value of blood pressure measured from a second location. In an example, the biometric sensors can comprise both electromagnetic energy sensors and blood pressure sensors.

In an example, a method can comprise using machine learning and/or artificial intelligence to adjust the operating parameters of an medical device implanted in a person in response to the person's blood flow as measured by a combination of internal biometric sensors implanted in the person and external biometric sensors worn by the person. In an example, a closed loop system or method can adjust the operation of a central (heart-assist) blood pump or a non-central (peripheral) blood pump in response to respiration rate in one or more ways selected from the group consisting of: activation of the device to change blood flow; adjusted device pumping volume to change blood flow; adjusted device rotation and/or speed to change blood flow; changed duration of device operation to change blood flow; selective activation of a sub-set of non-central (peripheral) blood pumps to change blood flow in a selected body location.

In an example, a method can comprise using machine learning (ML) to adjust operating parameters of an implanted medical device based on biometric indicators measured by implanted and wearable sensors. In an example, the operation of an implanted central (heart-assist) blood pump can be adjusted based on multivariate analysis of data concerning biometric indicator levels from a plurality of wearable devices worn at different locations on a person's body. In an example, a method can comprise using time-series analysis (TSA) to adjust operating parameters of an implanted medical device based on biometric indicators measured by implanted and wearable sensors. In an example, a method can comprise using principal components analysis (PCA) to adjust operating parameters of an implanted medical device based on biometric indicators measured by implanted and wearable sensors.

In an example, a method can analyze differences and/or variation in the values of a biometric indicator measured from both a person's arm and leg. In an example, a method can analyze differences and/or variation in the values of a biometric indicator measured from both a person's arm and torso. In an example, a method can analyze differences and/or variation in the values of a biometric indicator measured from both a person's leg and finger. In an example, a system can comprise a plurality of wearable devices with biometric sensors which are worn at different external locations on a person's body, a plurality of implanted medical devices which are in different internal locations within a person's body, wherein the operating parameters of a medical device are adjusted primarily (or entirely) based on data from the closest wearable device.

In an example, a method can comprise using hidden Markov model based machine learning to identify the relationships between the operating parameters of an implanted medical device and the values of biometric indicators measured by (implanted and wearable) sensors. In an example, a method can comprise using hierarchical-based machine learning to identify the relationships between the operating parameters of an implanted medical device and the values of biometric indicators measured by (implanted and wearable) sensors. In an example, a method can comprise using K-means based machine learning to identify the relationships between the operating parameters of an implanted medical device and the values of biometric indicators measured by (implanted and wearable) sensors.

In an example, a method can comprise using a random forest model to adjust operating parameters of an implanted medical device based on biometric indicators measured by implanted and wearable sensors. In an example, a method can comprise using a probit model to adjust operating parameters of an implanted medical device based on biometric indicators measured by implanted and wearable sensors. In an example, a method can comprise using a Markov model to adjust operating parameters of an implanted medical device based on biometric indicators measured by implanted and wearable sensors. In an example, a method can comprise using a Bayesian network, filter, or other Bayesian analysis method to adjust operating parameters of an implanted medical device based on biometric indicators measured by implanted and wearable sensors.

In an example, a method for adjusting and/or controlling the operating parameters of an implanted medical device can include analysis of a person's electromagnetic brain activity. In an example, an electromagnetic brain activity sensor can measure voltage fluctuations resulting from ionic current within the neurons of the brain. In an example, abnormal body tissue and/or fluid carbon dioxide levels (or changes in those levels) can trigger changes in repeating patterns and/or transient patterns of electromagnetic brain activity. In an example, abnormal body tissue and/or fluid glucose levels (or changes in those levels) can trigger changes in repeating patterns and/or transient patterns of electromagnetic brain activity. In an example, abnormal body tissue and/or fluid lactate (and/or lactic acid) levels (or changes in those levels) can trigger changes in repeating patterns and/or transient patterns of electromagnetic brain activity.

In an example, the operation of an implanted cardiac management device can be adjusted based on whole-body activity level as measured by a plurality of electromyographic (EMG) sensors. In an example, the operation of an implanted cardiac management device can be adjusted based on detection of a selected pattern of electromagnetic neuromuscular activity. In an example, operating parameters of an implanted medical device can be automatically adjusted based on heart sound patterns. In an example, operating parameters of an implanted medical device can be automatically adjusted based on cardiac rhythm or change in rhythm. In an example, operating parameters of an implanted medical device can be automatically adjusted based on cardiac conduction patterns or change in conduction patterns.

In an example, one of the biometric indicators which is monitored by this method can be lactate or lactic acid level within a person's body. In an example, the operation of an implanted medical device can be adjusted in response to abnormal lactate and/or lactic acid levels. In an example, a method can adjust parameters of cardiac functioning in response to high lactate (and/or lactic acid) levels detected by a wearable sensor. In an example, an electromagnetic muscle activity sensor can be configured to collect data concerning electromagnetic neuromuscular activity which is affected by muscle tissue lactate (and/or lactic acid) levels or changes in those levels.

In an example, an implanted medical device can deliver peripheral nerve stimulation. In an example, a method can comprise using machine learning and/or artificial intelligence to analyze one or more biometric indicators and, in response, adjust the operating parameters of a neurostimulation or neuromodulation device. In an example, a method can comprise using machine learning and/or artificial intelligence to analyze biometric indicators measured by a combination of implanted and wearable sensors and, in response, adjust the operating parameters of a neurostimulation or neuromodulation device.

In an example, the operation of an implanted cardiac function device can be adjusted based on whether a person is indoors or outdoors. In an example, the operation of an implanted cardiac function device can be adjusted based on environmental (and/or ambient) electromagnetic radiation levels and/or types. In an example, it can be advantageous for optimal operation of an implanted cardiac function device to be different in different environmental settings and/or conditions. In an example, a system can include one or more components selected from the group consisting of: food consumption sensor, microfluidic sensor, smart phone, voice recognition interface, air quality sensor, capacitance hygrometry sensor, electric motor, goniometer, motor, speech recognition interface, wireless data transmitter, brain activity sensor, data processor, environmental oxygen level sensor, Micro Electro Mechanical System (MEMS), pulse rate sensor, touch screen, ambient light sensor, and carbon monoxide sensor.

In an example, the human brain can function as a biological organic sensor for monitoring biological and/or physiological processes. In an example, a repeating electromagnetic brain activity pattern can be an oscillatory pattern. In an example, a system can increase the frequency, regularity, magnitude, and/or coordination of heart muscle contractions via an implanted cardiac management device when high lactate (and/or lactic acid) levels are detected via data from a wearable electromagnetic brain activity sensor. In an example, a system can increase the frequency of a heart beats via an implanted cardiac management device when low oxygen levels are detected in body tissue and/or fluid via data from a wearable electromagnetic brain activity sensor.

In an example, the operation of the implanted cardiac management device can be adjusted based on detected tissue and/or blood or tissue oxygen levels (or changes in those levels). In an example, the operation of an implanted cardiac function device can be adjusted based on ambient air composition, air quality, oxygen level, carbon dioxide level, carbon monoxide level, air-borne pollution and/or toxins, air borne allergens, and air speed. In this manner, the person's cardiac functioning can be adjusted based on detected tissue and/or blood or tissue oxygen levels (or changes in those levels). These changed patterns are detected by analysis of data from one or more wearable electromagnetic energy sensors, which triggers adjustment of cardiac function (via the implanted cardiac management device) which, in turn, restores normal body tissue and/or fluid oxygen levels. In an example, a system or method can comprise an integrated system for managing cardiac rhythm including a wearable device that measures body oxygen levels and an implanted cardiac rhythm management device.

In an example, one or more sensors which are part of a system or method for adjusting the operating parameters of an implanted medical device can be selected from the group consisting of: impedance sensor, piezoresistive sensor, sweat sensor, biochemical sensor, compass, electrophoresis sensor, inclinometer, pneumography sensor, temperature sensor, ambient temperature sensor, cardiotachometer, electromagnetic actuator, home automation control system, oximeter, blood glucose sensor, computer-to-human interface, energy transducer to generate energy from ambient electromagnetic energy, and inertial sensor.

In an example, machine learning and/or artificial intelligence can be used to analyze, identify, and quantify causal relationships between the operating parameters of an implanted cardiac rhythm device and lagged selected biometric indicators (e.g. blood or tissue oxygenation) during a first period of time; and then apply these quantified causal relationships during a second period of time to adjust these operating parameters to change these biometric indicators (e.g. blood or tissue oxygenation) in a desired direction. In an example, a system or method can increase the frequency of a person's heart beats via an implanted cardiac management device based on low heart rate detected via a wearable biometric sensor. In an example, a system or method can increase (or decrease) the frequency of a person's heart beats and/or the magnitude of a person's heart contractions in response to an increase (or decrease) in the person's respiration rate as detected by a wearable biometric sensor.

In an example, a method for moving biometric indicators (e.g. blood or tissue oxygenation) in a desired direction and/or into a desired range of values can comprise receiving data concerning operating parameters of a cardiac rhythm device implanted in a person during a first period of time; receiving data concerning the values of one or more biometric indicators (e.g. blood or tissue oxygenation) for that person from a combination of implanted sensors and wearable sensors during the first time period; using machine learning and/or artificial intelligence to identify the lagged effects of the operating parameters on the biometric indicators (e.g. blood or tissue oxygenation) during the first time period; receiving data concerning the values of the biometric indicators (e.g. blood or tissue oxygenation) for that person from a combination of implanted sensors and wearable sensors during a second period; and using machine learning and/or artificial intelligence to evaluate the values of the biometric indicators during the second period and the previously-identified effects in order to determine how the operating parameters of the implanted cardiac rhythm device should be changed in order to change the one or more biometric indicators (e.g. blood or tissue oxygenation) to improve the person's health.

In an example, a method for moving biometric indicators (e.g. blood or tissue oxygenation) in a desired direction and/or into a desired range of values can comprise receiving data concerning operating parameters of a cardiac rhythm device implanted in a person during a first period of time; receiving data concerning the values of one or more biometric indicators (e.g. blood or tissue oxygenation) for that person from a combination of implanted sensors and wearable sensors during the first time period; using machine learning and/or artificial intelligence to identify the lagged effects of the operating parameters on the biometric indicators (e.g. blood or tissue oxygenation) during the first time period; receiving data concerning the values of the biometric indicators (e.g. blood or tissue oxygenation) for that person from a combination of implanted sensors and wearable sensors during a second period; evaluating whether the values are within a normal and/or healthy range; and if the values are not within the normal and/or healthy range, using machine learning and/or artificial intelligence to determine how the operating parameters of the implanted cardiac rhythm device should be changed in order to move the values into the normal and/or healthy range.

In an example, a biometric indicator which is measured and managed by a system can be body oxygenation level or changes in body oxygenation levels. In an example, operation of an implanted circulatory assistance device can be controlled and/or adjusted based on body oxygenation level or changes in body oxygenation levels. The ability to measure oxygen levels via sensors at one or more peripheral locations can provide more accurate measures of body-wide oxygenation than, for example, a single central sensor. The ability to measure oxygen levels in the brain (relatively directly) can provide a more accurate and timely measure of brain oxygenation than a limb-worn sensor or centrally-implanted sensor.

In an example, human circulatory assistance can be provided by an implanted central (heart assist) blood pump whose operation is adjusted in real time based on overall body oxygenation level. In an example, the operation of an implanted blood pump in a given location can be selectively activated and/or adjusted when the value of body oxygenation level measured from that location is abnormal for at least a given length of time, wherein this length of time is selected from within a range of 10 seconds to 10 minutes. In an example, one or more oxygen-related biometric indicators can be selected from the group consisting of: arterial oxygen saturation level, oxygen metabolism level, saturation of peripheral oxygen, brain oxygenation level, and peripheral tissue oxygenation level. In an example, the operating parameters of an implanted medical device can be automatically adjusted based on peripheral oxygen saturation (SpO2) level or arterial oxygen saturation (SaO2) level as measured by external (e.g. wearable) and/or internal (e.g. implanted) oxygen sensors.

In an example, a closed loop system or method can adjust the operation of a central (heart-assist) blood pump or a non-central (peripheral) blood pump in response to an abnormal body hydration level in one or more ways selected from the group consisting of: activation of the device to change blood flow; adjusted device pumping volume to change blood flow; adjusted device rotation and/or speed to change blood flow; changed duration of device operation to change blood flow; selective activation of a sub-set of non-central (peripheral) blood pumps to change blood flow in a selected body location. In an example, if the brain detects low tissue oxygen levels, then this changes electromagnetic brain activity patterns, which is then detected by a wearable electromagnetic energy sensor, which then adjusts the operation of the implanted cardiac management device, which then increases blood flow, which can then restore proper tissue oxygen levels.

In an example, a system can further comprise one or more components selected from the group consisting of: accelerometer, augmented reality eyewear, chemiresistor, electromagnetic energy sensor, human-to-computer interface, photodetector, acoustic energy sensor, breathing rate sensor, digital camera, galvanic skin response (GSR) sensor, microphone, solar panel, VR eyewear, brain oxygenation sensor, deely bobbers, eye muscle (EOG) sensor, microchip, skin conductance sensor, vibrating component, and drug pump. A system for circulatory assistance with a plurality of implanted blood pumps can help to maintain proper and consistent blood circulation (and body oxygenation) in all regions of the body. A system for selective circulatory assistance with a plurality of individually-controllable implanted blood pumps whose operation is adjusted (in real time) based on associated wearable oxygenation sensors.

If a person's brain detects low tissue oxygen levels, then this changes electromagnetic brain activity patterns, which is then detected by a wearable electromagnetic energy sensor, which then adjusts the operation of the implanted cardiac management device, which then increases blood flow, which can then restore proper tissue oxygen levels. In an example, a system can increase the frequency, regularity, magnitude, and/or coordination of a person's heart muscle contractions via an implanted cardiac management device based on low oxygen levels detected using a wearable spectroscopic sensor or EEG sensor. For example, detection of a low oxygen levels in the brain by a wearable electromagnetic energy sensor can trigger increased blood flow which, in turn, can help to restore proper oxygen levels for the brain.

In an example, a method can comprise using machine learning and/or artificial intelligence to adjust the operating parameters of an medical device implanted in a person in response to the person's blood or tissue oxygen level as measured by a combination of internal biometric sensors implanted in the person and external biometric sensors worn by the person. In an example, detection of an abnormal body oxygenation level by wearable sensors can trigger increased systemic blood flow via changing the operating parameters of an implanted medical device. In an example, the value of body oxygenation level can be considered abnormal when it is at least X % lower than the minimum value in a benchmark range of values, wherein X % is selected from within a range of 10% to 50%. In an example, a system can increase the frequency, regularity, magnitude, and/or coordination of heart muscle contractions via an implanted cardiac management device based on low oxygen levels detected using a wearable spectroscopic sensor.

In an example, biometric sensors which are part of a system or method can comprise oxygenation sensors. In an example, biometric sensors can comprise both blood pressure sensors and oxygenation sensors. In an example, a system can increase the magnitude of heart contractions via an implanted cardiac management device based on low oxygen levels detected in body tissue and/or fluid via a wearable biometric sensor. In an example, a system can increase the frequency of a heart beats via an implanted cardiac management device based on low oxygen levels detected in body tissue and/or fluid via a wearable spectroscopic sensor. In an example, a system can increase the frequency, regularity, magnitude, and/or coordination of a person's heart muscle contractions via an implanted cardiac management device based on low oxygen levels detected using a wearable spectroscopic sensor.

In an example, a biometric indicator can be body oxygenation level. In an example, a method can comprise detection of low oxygen levels in peripheral tissue (or organs) by a wearable biometric sensor, wherein this triggers a change in the operation of an implanted medical device to increase blood flow, which in turn helps to restore proper oxygen levels for that tissue (or organs). In an example, a system can adjust parameters of cardiac functioning in response to low oxygen levels which are detected by a wearable biometric sensor (such as a wearable spectroscopic sensor). In an example, the wearable component of the system can be a finger ring and the measured analyte can be oxygen level.

In an example, a system can increase the frequency, regularity, magnitude, and/or coordination of heart muscle contractions via an implanted cardiac management device based on low oxygen levels detected using a wearable light energy sensor or electromagnetic energy sensor. In an example, a system can increase the magnitude of a person's heart contractions via an implanted cardiac management device based on high carbon dioxide levels detected in the person's body tissue and/or fluid via a wearable spectroscopic sensor. In an example, a system can increase the magnitude of a person's heart contractions via an implanted cardiac management device based on high carbon dioxide levels detected in the person's body tissue and/or fluid via a wearable biometric sensor.

Although the analogy is not perfect, a closed loop system for circulatory assistance with a plurality of implanted blood pumps whose operations are selectively controlled and/or adjusted by a plurality of wearable biometric sensors is analogous to having a climate control system for a home or other building with different HVAC (e.g. heating or cooling) zones in different areas throughout the home or other building. In an example, a system for human circulatory assistance can comprise a plurality of wearable devices and plurality of implanted non-central (peripheral) blood pumps which enables independent adjustment of circulatory assistance for different portions of a person's body based on biometric indicator values from those different body portions.

In an example, this method can comprise adjusting the operating parameters of an implantable drug delivery system based on data from a wearable glucose sensor. In an example, this method can comprise adjusting the operating parameters of an implantable drug delivery system based on data from a wearable eating detector. In an example, a method can comprise using machine learning and/or artificial intelligence to analyze one or more biometric indicators and, in response, adjust the operating parameters of an implantable drug pump. In an example, this method can comprise adjusting the operating parameters of an implantable drug delivery system based on data from a blood glucose level sensor. In an example, the operation of the implanted cardiac management device can be adjusted based on detected glucose levels and/or changes in glucose levels.

In an example, a method can comprise using machine learning and/or artificial intelligence to analyze biometric indicators measured by a combination of implanted and wearable sensors and, in response, adjust the operating parameters of an implantable cardiac rhythm management device. In an example, a method can comprise using machine learning and/or artificial intelligence to analyze biometric indicators measured by a combination of implanted and wearable sensors and, in response, adjust the operating parameters of an implantable cardioverter defibrillator (ICD). In an example, a method can comprise using machine learning and/or artificial intelligence to analyze biometric indicators measured by a combination of implanted and wearable sensors and, in response, adjust the operating parameters of an implantable drug pump.

In an example, a system can comprise one or more wearable pressure sensors which measure a person's blood pressure. In an example, a closed loop system for adjusting the operation of a central (heart-assist) blood pump or a non-central (peripheral) blood pump can adjust the parameters of such a device in response to abnormal blood pressure. In an example, the flow rate from an implanted blood pump can be adjusted in response to an abnormally-high blood pressure detected by a wearable device. In an example, the flow rate from an implanted blood pump can be adjusted in response to an abnormally-low blood pressure detected by a wearable device. In an example, data concerning one or more biometric indicators can be obtained from sensors on a cuff or sleeve which is worn by a person.

In an example, an implanted medical device whose operation is adjusted by this method can be an implanted blood pump. In an example, a blood pump can produce continuous blood flow. In an example, a blood pump can produce variable and/or pulsatile blood flow. In an example, an implanted medical device whose operation is adjusted by this method can be an implanted circulatory assist device. In an example, machine learning and/or artificial intelligence can be used to adjust the flow rate of implanted blood pump based on data from internal (implanted) and external (wearable) sensors. In an example, an implanted blood pump can comprise a rotating impellor, wherein the rotational speed of the impellor is adjusted based on the values of biometric indicators received from these sensors.

In an example, a system or method can increase (or decrease) the frequency of a person's heart beats via an implanted cardiac management device based on low (or high) blood pressure detected via a wearable spectroscopic sensor. In an example, a spectroscopic sensor can be configured to receive light energy which has been reflected from, or passed through, aqueous humour, blood, blood vessels, body fat, brain tissue, dermis, ear drum, earlobe, epidermis, fat tissue, intercellular fluid, lung tissue, lymphatic fluid, lymphatic passageways, muscle tissue, nerve tissue, blood, saliva, skin, sweat, and/or tears in order to monitor for troponin and/or changes in troponin. In an example, a first subset of spectroscopic sensors in a plurality of spectroscopic sensors can receive light beams reflected from a first tissue depth and a second subset of spectroscopic sensors in the plurality of spectroscopic sensors can receive light beams reflected from a second tissue depth. In an example, a spectroscopic sensor can directed light after it has been reflected from, or passed through, the person's body tissue and/or fluid. In an example, a wearable device can have an ion mobility spectroscopic sensor.

In an example, a system can increase the frequency, regularity, magnitude, and/or coordination of a person's heart muscle contractions via an implanted cardiac management device based on high carbon dioxide levels detected in the person's body tissue and/or fluid via a wearable spectroscopic sensor, EMG sensor, or EEG sensor. In an example, a system or method can increase (or decrease) the frequency of a person's heart beats via an implanted cardiac management device based on low (or high) blood pressure detected via a wearable biometric sensor. In an example, a system can increase the frequency of a heart beats via an implanted cardiac management device when high lactate (and/or lactic acid) levels are detected in body tissue and/or fluid via data from a wearable electromagnetic brain activity sensor. In an example, a system can increase the frequency, regularity, magnitude, and/or coordination of a person's heart muscle contractions via an implanted cardiac management device based on high lactate levels detected using a wearable spectroscopic sensor or EEG sensor.

In an example, the operation of an implanted cardiac management device can be adjusted based on detected heart rate and/or changes in heart rate. In an example, a biometric indicator which is measured and managed by a system can be (peripherally measured) heart rate variability. In an example, a closed-loop system for adjusting operating parameters of an implanted medical device can include a heart rate sensor. In an example, operating parameters of an implanted medical device can be automatically adjusted based on heart rate, change in heart rate, or variability over time. photoplethysmography (PPG) is a method which analyzes variation in the amount (and spectral distribution) of light which is caused by reflection of the light from (or transmission of the light through) body tissue in order to measure one or more biometric indicators (such as heart rate or heart rate variation). In an example, a system can increase the frequency of a person's heart beats via an implanted cardiac management device based on low heart rate detected via a wearable spectroscopic sensor.

In an example, a system or method can increase (or decrease) the frequency, regularity, magnitude, and/or coordination of a person's heart muscle contractions via an implanted cardiac management device based on low (or high) glucose levels detected in the person's body tissue and/or fluid via a wearable spectroscopic sensor, tissue impedance sensor, or EEG sensor. In an example, a system can increase the frequency, regularity, magnitude, and/or coordination of a person's heart muscle contractions via an implanted cardiac management device based on a low (or high) heart rate level detected in the person's body tissue and/or fluid via a wearable spectroscopic sensor or EEG sensor. In an example, a system can adjust the frequency, regularity, magnitude, and/or coordination of heart muscle contractions via an implanted cardiac management device based on a person's sleep status and/or stage of sleep.

In an example, a system can increase the frequency, regularity, magnitude, and/or coordination of a person's heart muscle contractions via an implanted cardiac management device based on a low (or high) heart rate level detected in the person's body tissue and/or fluid via a wearable spectroscopic sensor. In an example, a system can increase the frequency, regularity, magnitude, and/or coordination of a person's heart muscle contractions via an implanted cardiac management device based on a low heart rate level detected in the person's body tissue and/or fluid via a wearable spectroscopic sensor. In an example, a system can increase the frequency, regularity, magnitude, and/or coordination of a person's heart muscle contractions via an implanted cardiac management device based on high lactate levels detected using a wearable spectroscopic sensor. In an example, a system can increase the magnitude of a person's heart contractions via an implanted cardiac management device based on high lactate levels detected using a wearable spectroscopic sensor.

In an example, a system can increase (or decrease) the frequency, regularity, magnitude, and/or coordination of a person's heart muscle contractions via an implanted cardiac management device based on a low (or high) blood pressure level detected in the person's body tissue and/or fluid via a wearable spectroscopic sensor. In an example, a system can increase (or decrease) the magnitude of a person's heart contractions via an implanted cardiac management device based on a low (or high) blood pressure level in the person's body tissue and/or fluid via a wearable spectroscopic sensor. In an example, a system can increase the frequency of a person's heart beats via an implanted cardiac management device based on high carbon dioxide levels detected in the person's body tissue and/or fluid via a wearable spectroscopic sensor. In an example, a system can increase the frequency of a person's heart beats via an implanted cardiac management device based on high lactate levels detected in the person's body tissue and/or fluid via a wearable spectroscopic sensor.

In an example, a spectroscopic sensor of a system can include a first light emitter and a second light emitter. In an example, a spectroscopic sensor of a system can include one or more light (energy) emitters. In an example, a spectroscopic sensor of a system can include an array of light (energy) emitters. A spectroscopic sensor collects data concerning the spectrum of light energy which has been reflected from (or has passed through) body tissue, organs, and/or fluid. In an example, this device can comprise both spectroscopic and microwave energy sensors for measuring a biometric indicator value in the body. In an example, a system can increase (or decrease) the frequency, regularity, magnitude, and/or coordination of a person's heart muscle contractions via an implanted cardiac management device based on a low (or high) blood pressure level detected in the person's body tissue and/or fluid via a wearable spectroscopic sensor or EEG sensor.

In an example, a system or method can decrease the frequency, regularity, magnitude, and/or coordination of heart muscle contractions via an implanted cardiac management device when a low whole-body activity level is detected via data from an electromagnetic muscle activity sensor. In an example, a system can increase the frequency, regularity, magnitude, and/or coordination of a person's heart muscle contractions via an implanted cardiac management device based on high carbon dioxide levels detected in the person's body tissue and/or fluid via a wearable spectroscopic sensor. In an example, a system can increase the frequency, regularity, magnitude, and/or coordination of a person's heart muscle contractions via an implanted cardiac management device based on high lactate levels detected in the person's body tissue and/or fluid via a wearable spectroscopic sensor. In an example, a system can increase the frequency, regularity, magnitude, and/or coordination of a person's heart muscle contractions via an implanted cardiac management device based on high lactate levels detected in the person's body tissue and/or fluid via a wearable light energy or electromagnetic energy sensor.

In an example, a method can comprise receiving biometric data from a respiration sensor. In an example, a wearable device can comprise a motion sensor, spectroscopic sensor, or electromagnetic energy sensor which measures respiration rate. In an example, a person's respiration rate can be measured by reflecting light energy from (and/or passing light energy through) body tissue. In an example, a closed-loop system for adjusting operating parameters of an implanted medical device can include a respiration sensor. In an example, respiration rate can be measured from multiple locations on the person's body.

In an example, a closed-loop system for adjusting operating parameters of an implanted medical device can include an eating detector. In an example, a method can comprise receiving biometric data from an eating detector. In an example, the frequency, color, and/or spectrum of a beam of light emitted from a light emitter can be changed in response to specific environmental conditions (e.g. temperature or humidity) and/or specific activities in which the person wearing a system is engaged (e.g. high level of movement, eating, sleeping, etc.) in order to more accurately measure a biometric indicator value in the body. In an example, a system can include one or more wearable sensors selected from the group consisting of blood glucose sensor, camera, chemical sensor, heart rate sensor, humidity and moisture sensor, eating detector, and respiration sensor.

In an example, a method can comprise using logit analysis to adjust operating parameters of an implanted medical device based on biometric indicators measured by implanted and wearable sensors. In an example, a method can comprise using spectroscopic analysis to adjust operating parameters of an implanted medical device based on biometric indicators measured by implanted and wearable sensors. In an example, a method can comprise using carlavian curve analysis to adjust operating parameters of an implanted medical device based on biometric indicators measured by implanted and wearable sensors. In an example, a method can comprise using power spectrum analysis to adjust operating parameters of an implanted medical device based on biometric indicators measured by implanted and wearable sensors.

In an example, a method for adjusting the operation of an implanted medical device within a person can comprise using machine learning and/or artificial intelligence to analyze data from a plurality of spectroscopic sensors worn on that person. In an example, the plurality of spectroscopic sensors can be distributed around at least 50% of the circumference of a band worn on the person's wrist, ankle, finger, arm, or leg. In an example, these spectroscopic sensor can direct light toward the person's body, receive this light after it has been reflected from (or transmitted through) the person's body tissue, and measure one or more biometric indicators based on changes in the spectral distribution of the light caused by interaction with the body tissue. In an example, these spectroscopic sensors can emit both coherent and non-coherent light.

In an example, a system can include one or more wearable sensors selected from the group consisting of an optical sensor, a near infrared spectroscopy (NIRS) sensor, a spectroscopic sensor, and a PhotoPleythysmoGraph (PPG) sensor. In an example, a method can comprise receiving biometric data from an optical sensor, near infrared spectroscopy (NIRS) sensor, spectroscopic sensor, and/or PhotoPleythysmoGraph (PPG) sensor. In an example, a closed-loop system for adjusting operating parameters of an implanted medical device can include an optical sensor, near infrared spectroscopy (NIRS) sensor, spectroscopic sensor, and/or PhotoPleythysmoGraph (PPG) sensor.

In an example, a light emitter and a light receiver together can comprise a spectroscopic (or spectroscopy) sensor. In an example, different emitters in this array can emit light at different wavelengths and/or with different light spectral distributions. In an example, different receivers in this array can receive light at different wavelengths and/or with different light spectral distributions. In an example, a light emitter can automatically cycle through light emissions with a variety of wavelengths (or wavelength ranges or spectral distributions) during different time periods in order to measure different physiological parameters, analytes, or conditions. In an example, machine learning and/or AI for adjusting operating parameters of an implanted medical device can employ spectroscopic analysis (SA).

In an example, a sensor whose data is used to adjust the operating parameters of an implanted medical device can be a spectroscopy sensor. A spectroscopy sensor can comprise a light emitter (e.g. a light source) and a light receiver (e.g. a photodetector). In an example, a light receiver can emit near-infrared light. In an example, a first sensor comprising a first light emitter and a first light receiver can reflect light from a body surface at a first angle and a second sensor comprising a second light emitter and a second light receiver can reflect light from the body surface at a second angle. This can enable analysis of tissue composition at different depths. In an example, a sensor whose data is used to adjust the operating parameters of an implanted device can be an Inertial Motion unit (IMU). An IMU can comprise an accelerometer, a gyroscope, and/or an inclinometer. In an example, an IMU can measure a person's body activity and/or exercise level. In an example, a sensor can be an ECG sensor or an EEG sensor.

In an example, data concerning one or more biometric indicators can be obtained from a combination of internal (e.g. implanted) sensors and external (e.g. wearable) sensors on a shirt (e.g. smart shirt with imbedded sensors) which is worn by the person. In an example, data concerning one or more biometric indicators can be obtained from a combination of internal (e.g. implanted) sensors and external (e.g. wearable) sensors on a necklace which is worn by the person. In an example, data concerning one or more biometric indicators can be obtained from a combination of internal (e.g. implanted) sensors and external (e.g. wearable) sensors on a finger ring which is worn by the person. In an example, data concerning one or more biometric indicators can be obtained from a combination of internal (e.g. implanted) sensors and external (e.g. wearable) sensors on a cuff or sleeve which is worn by the person.

In an example, a method can comprise using machine learning and/or artificial intelligence to adjust the operating parameters of an medical device implanted in a person in response to the person's body temperature as measured by a combination of internal biometric sensors implanted in the person and external biometric sensors worn by the person. In an example, a method can comprise using machine learning and/or artificial intelligence to adjust the operating parameters of an medical device implanted in a person in response to the person's heart sounds as measured by a combination of internal biometric sensors implanted in the person and external biometric sensors worn by the person.

In an example, a closed-loop system for continuous adjustment of the operating parameters of an implanted medical device can include one or more optical sensors on a wearable device. These optical sensors can comprise paired light emitters and light receivers, light energy from a light emitter is received by a light receiver after that light energy has been transmitted through or reflected by body tissue and/or fluid. In an example, a light receiver receives this light after it has been reflected from, or passed through, a person's body tissue and/or fluid. In an example, a light emitter can emit a sequence of light pulses at different selected frequencies over time.

In an example, a method can comprise using variance to adjust operating parameters of an implanted medical device based on biometric indicators measured by implanted and wearable sensors. In an example, a method can comprise using orthogonal transformation to adjust operating parameters of an implanted medical device based on biometric indicators measured by implanted and wearable sensors. In an example, a method can comprise using non-linear programming to adjust operating parameters of an implanted medical device based on biometric indicators measured by implanted and wearable sensors. In an example, a method can comprise using multivariate logit or probit to adjust operating parameters of an implanted medical device based on biometric indicators measured by implanted and wearable sensors.

In an example, a method can comprise using reinforcement-based machine learning to identify the relationships between the operating parameters of an implanted medical device and the values of biometric indicators measured by (implanted and wearable) sensors. In an example, a method can comprise using supervised machine learning to identify the relationships between the operating parameters of an implanted medical device and the values of biometric indicators measured by (implanted and wearable) sensors. In an example, a method can comprise using support vector machine based machine learning to identify the relationships between the operating parameters of an implanted medical device and the values of biometric indicators measured by (implanted and wearable) sensors. In an example, a method can comprise using unsupervised machine learning to identify the relationships between the operating parameters of an implanted medical device and the values of biometric indicators measured by (implanted and wearable) sensors.

In an example, a method can comprise using nearest neighbor based machine learning to identify the relationships between the operating parameters of an implanted medical device and the values of biometric indicators measured by (implanted and wearable) sensors. In an example, a method can comprise using neural networks based machine learning to identify the relationships between the operating parameters of an implanted medical device and the values of biometric indicators measured by (implanted and wearable) sensors. In an example, a method can comprise using regression-based machine learning to identify the relationships between the operating parameters of an implanted medical device and the values of biometric indicators measured by (implanted and wearable) sensors.

In an example, a method can comprise using machine learning and/or artificial intelligence to identify person-specific relationships between the operating parameters of a medical device implanted in a person's body and biometric indicators measured by sensors worn on the person's body in historical clinical information for that person. In an example, a method can comprise using machine learning and/or artificial intelligence to identify a normal range for a biometric indicator for a specific person. In an example, a method can comprise using machine learning and/or artificial intelligence to analyze data from a sensor worn by a person to identify a normal range for a biometric indicator for that person.

In an example, a method can comprise using machine learning and/or artificial intelligence to adjust the operating parameters of an medical device implanted in a person in response to the person's pulse rate and/or amplitude as measured by a combination of internal biometric sensors implanted in the person and external biometric sensors worn by the person. In an example, a method can comprise using machine learning and/or artificial intelligence to adjust the operating parameters of an medical device implanted in a person in response to the person's food consumption as measured by a combination of internal biometric sensors implanted in the person and external biometric sensors worn by the person. In an example, a method can comprise using machine learning and/or artificial intelligence to adjust the operating parameters of an medical device implanted in a person in response to the person's blood glucose level as measured by a combination of internal biometric sensors implanted in the person and external biometric sensors worn by the person.

In an example, a method can comprise using least squares estimation to adjust operating parameters of an implanted medical device based on biometric indicators measured by implanted and wearable sensors. In an example, a method can comprise using Fourier transformation to adjust operating parameters of an implanted medical device based on biometric indicators measured by implanted and wearable sensors. In an example, a method can comprise using eigenvalue decomposition to adjust operating parameters of an implanted medical device based on biometric indicators measured by implanted and wearable sensors.

In an example, a method can comprise using fuzzy C-means based machine learning to identify the relationships between the operating parameters of an implanted medical device and the values of biometric indicators measured by (implanted and wearable) sensors. In an example, a method can comprise using Gaussian process based machine learning to identify the relationships between the operating parameters of an implanted medical device and the values of biometric indicators measured by (implanted and wearable) sensors. In an example, a method can comprise using GLM-based machine learning to identify the relationships between the operating parameters of an implanted medical device and the values of biometric indicators measured by (implanted and wearable) sensors.

In an example, a method can comprise using decision tree learning to adjust operating parameters of an implanted medical device based on biometric indicators measured by implanted and wearable sensors. In an example, a method can comprise using data mining to adjust operating parameters of an implanted medical device based on biometric indicators measured by implanted and wearable sensors. In an example, a method can comprise using correlation to adjust operating parameters of an implanted medical device based on biometric indicators measured by implanted and wearable sensors. In an example, a method can comprise using auto-regression to adjust operating parameters of an implanted medical device based on biometric indicators measured by implanted and wearable sensors. In an example, a method can comprise using a support vector machine to adjust operating parameters of an implanted medical device based on biometric indicators measured by implanted and wearable sensors. In an example, a method can comprise using a Kalman filter to adjust operating parameters of an implanted medical device based on biometric indicators measured by implanted and wearable sensors.

In an example, a method can comprise using decision tree based machine learning to identify the relationships between the operating parameters of an implanted medical device and the values of biometric indicators measured by (implanted and wearable) sensors. In an example, a method can comprise using discriminant analysis based machine learning to identify the relationships between the operating parameters of an implanted medical device and the values of biometric indicators measured by (implanted and wearable) sensors. In an example, a method can comprise using ensemble methods based machine learning to identify the relationships between the operating parameters of an implanted medical device and the values of biometric indicators measured by (implanted and wearable) sensors.

In an example, a method can comprise using Baysian-based machine learning to identify the relationships between the operating parameters of an implanted medical device and the values of biometric indicators measured by (implanted and wearable) sensors. In an example, a method can comprise using classification-based machine learning to identify the relationships between the operating parameters of an implanted medical device and the values of biometric indicators measured by (implanted and wearable) sensors. In an example, a method can comprise using clustering to identify the relationships between the operating parameters of an implanted medical device and the values of biometric indicators measured by (implanted and wearable) sensors.

In an example, a method can comprise changing the operating parameters of an implanted medical device can comprise: receiving a first value of a biometric indicator during a first period of time from a sensor on a device worn by a person; if the first value is not within a normal range for that indicators, adjusting an operating parameter of an implanted medical device within the person by a first amount during a second period of time based on the first value of the biometric indicator; receiving a second value of the biometric indicator during a third period of time; and if the second value is not within the normal range for that indicator, adjusting the operating parameter by a second amount during a fourth period of time.

In an example, a method can comprise changing the operating parameters of an implanted medical device can comprise: receiving a first value of a biometric indicator during a first period of time from a sensor on a device worn by a person; if the first value is not within a normal range for that indicators, adjusting an operating parameter of an implanted medical device within the person by a first amount during a second period of time based on the first value of the biometric indicator; receiving a second value of the biometric indicator during a third period of time; and if the second value is not within the normal range for that indicator, adjusting the operating parameter by a second amount during a fourth period of time, wherein the second amount is less than the first amount.

In an example, a method can comprise changing the operating parameters of an implanted medical device can comprise: receiving a first value of a biometric indicator during a first period of time from a sensor on a device worn by a person; adjusting an operating parameter of an implanted medical device within the person by a first amount during a second period of time based on the first value of the biometric indicator; receiving a second value of the biometric indicator during a third period of time; and adjusting the operating parameter by a second amount during a fourth period of time, wherein the second amount is less than the first amount.

In an example, a method for adjusting and/or controlling the operating parameters of an implanted medical device can comprise: receiving data concerning biometric indicators from one or more sensors on the implanted medical device (implanted in a person's body); receiving data concerning biometric indicators from one or more sensors on a wearable device (worn by the person); analyzing the combined data from all of these sensors; and changing the operating parameters of the implanted medical device based on the results of this analysis.

In an example, a method for adjusting and/or controlling the operating parameters of an implanted medical device can comprise: receiving data concerning biometric indicators from one or more sensors on the implanted medical device (implanted in a person's body); receiving data concerning biometric indicators from one or more sensors on a generic wearable device (e.g. a generic smart watch); analyzing the combined data from all of these sensors; and changing the operating parameters of the implanted medical device based on the results of this analysis.

In an example, a method for adjusting and/or controlling the operating parameters of an implanted medical device can comprise: receiving data concerning biometric indicators from one or more sensors on the implanted medical device (implanted in a person's body); receiving data concerning biometric indicators from one or more sensors on a specialized wearable device (e.g. a wearable medical device); analyzing the combined data from all of these sensors; and changing the operating parameters of the implanted medical device based on the results of this analysis.

In an example, a method for adjusting and/or controlling an implanted medical device can comprise changing one or more operating parameters of the device based on multivariate statistical analysis of data from implanted biometric sensors and wearable biometric sensors. In an example, a method for adjusting and/or controlling an implanted medical device can comprise using machine learning and/or artificial intelligence to change one or more operating parameters of the device based on analysis of data from implanted biometric sensors and wearable biometric sensors.

In an example, a method for adjusting and/or controlling an implanted medical device can comprise changing one or more operating parameters of the device based on multivariate statistical analysis of data from implanted biometric sensors, wearable biometric sensors, and environmental sensors. In an example, a method for adjusting and/or controlling an implanted medical device can comprise using machine learning and/or artificial intelligence to change one or more operating parameters of the device based on analysis of data from implanted biometric sensors, wearable biometric sensors, and environmental sensors.

In an example, a method for adjusting and/or controlling an implanted medical device can comprise changing one or more operating parameters of the device based on multivariate statistical analysis of data from wearable biometric sensors and environmental sensors. In an example, a method for adjusting and/or controlling an implanted medical device can comprise using machine learning and/or artificial intelligence to change one or more operating parameters of the device based on analysis of data from wearable biometric sensors and environmental sensors.

In an example, a method for adjustment and/or control of an implanted medical device can comprise: using machine learning and/or artificial intelligence to adjust one or more operating parameters of the implanted medical device based on the level, the rate of change (e.g. slope), and/or the cyclical frequency of one or more biometric indicators. In an example, a method for adjustment and/or control of an implanted medical device can comprise: using machine learning and/or artificial intelligence to adjust one or more operating parameters of the implanted medical device based on the level, the rate of change (e.g. slope), and/or the cyclical frequency of one or more biometric indicators measured by a combination of implanted and wearable sensors.

In an example, a system can adjust the operating parameters of an implanted medical device based on the difference and/or variation between the values of a biometric indicator as measured by an implanted (e.g. internal) sensor and the values of that biometric indicator as measured by a wearable (e.g. external) sensor. In an example, a system can adjust the operating parameters of an implanted medical device based on the combined data from implanted (e.g. internal) biometric sensors and wearable (e.g. external) biometric sensors. In an example, a system can adjust the operating parameters of an implanted medical device based on the average of biometric indicator values measured by implanted (e.g. internal) biometric sensors and wearable (e.g. external) biometric sensors. In an example, a system can adjust the operating parameters of an implanted medical device based on interaction between biometric indicator values measured by implanted (e.g. internal) biometric sensors and wearable (e.g. external) biometric sensors.

In an example, data concerning one or more biometric indicators can be obtained from a combination of internal (e.g. implanted) sensors and external (e.g. wearable) sensors on a watch band (e.g. watch band with a plurality of sensors) which is worn by the person. In an example, data concerning one or more biometric indicators can be obtained from a combination of internal (e.g. implanted) sensors and external (e.g. wearable) sensors on a sock (e.g. smart sock with imbedded sensors) which is worn by the person. In an example, data concerning one or more biometric indicators can be obtained from a combination of internal (e.g. implanted) sensors and external (e.g. wearable) sensors on a smart watch which is worn by the person. In an example, data concerning one or more biometric indicators can be obtained from a combination of internal (e.g. implanted) sensors and external (e.g. wearable) sensors on a smart collar which is worn by the person.

In an example, data concerning one or more biometric indicators can be obtained from a combination of internal (e.g. implanted) sensors and external (e.g. wearable) sensors on a chest strap which is worn by the person. In an example, data concerning one or more biometric indicators can be obtained from a combination of internal (e.g. implanted) sensors and external (e.g. wearable) sensors on a bra (e.g. smart bra with imbedded sensors) which is worn by the person. In an example, data concerning one or more biometric indicators can be obtained from a combination of internal (e.g. implanted) sensors and external (e.g. wearable) sensors on a belt which is worn by the person.

In an example, one or more light receivers can be configured to receive light energy which has been reflected from, passed through, and/or scattered by body tissue, organs, and/or fluid. In an example, pairs of light emitters and light receivers can be distributed around the circumference of a wearable device (such as a finger ring, watch band, wrist band, arm band, or ankle band) such that at least one pair is in close contact with the surface of a person's body regardless of rotation and/or shifting of the wearable device. In an example, a plurality of light receivers can be configured in a circular or other arcuate array including a light emitter. In an example, a plurality of light receivers can be distributed around the circumference of a wearable device, being pair-wise separated from each other by 45, 60, 90, or 180 degrees. In an example, an implanted or wearable biometric sensor can comprise a rectangular array, grid, and/or matrix of light receivers. In an example, a wearable device can include a rectangular array, grid, and/or matrix of light receivers. In an example, a wearable device can include a three-dimensional stacked array, grid, and/or matrix of light receivers.

In an example, machine learning and/or artificial intelligence can be used to analyze, identify, and quantify causal relationships between the operating parameters of an implanted medical device and lagged selected biometric indicators during a first period of time; and then apply these quantified causal relationships during a second period of time to adjust these operating parameters to change these biometric indicators in a desired direction. In an example, machine learning and/or AI for adjusting operating parameters of an implanted medical device can employ power spectrum analysis (PSA). In an example, machine learning and/or AI for adjusting operating parameters of an implanted medical device can employ correlation.

In an example, a system can include a wireless data transmitter and/or data receiver. In an example, data can be transmitted wirelessly between an implanted medical device and a wearable device. In an example, data can be transmitted wirelessly between an implanted medical device and a wearable device via Bluetooth. In an example, data can be transmitted wirelessly between an implanted medical device and a wearable device via a local area network (LAN). In an example, this wireless transmission can occur using far-field communication and/or bovine communication from the far side.

In an example, data can be transmitted wirelessly from an implanted medical device and/or a wearable device via WIFI. In an example, data can be transmitted wirelessly from an implanted medical device and/or a wearable device via radio-frequency identification (RFID). In an example, data can be transmitted wirelessly from an implanted medical device and/or a wearable device via a wide area network (WAN). In an example, data can be transmitted wirelessly between an implanted medical device and a wearable device via radio-frequency identification (RFID). In an example, data can be transmitted wirelessly between an implanted medical device and a wearable device via near field communication (NFC). In an example, data can be transmitted wirelessly between an implanted medical device and a wearable device via a wide area network (WAN).

In an example, a method for adjusting and/or controlling the operation of an implanted medical device in a person can comprise: using machine learning and/or artificial intelligence to analyze data which includes the operating parameters of the implanted medical device over time and the values of one or more biometric indicators of that person over time in order to identify the size of the effects of changes in those parameters on changes in those biometric indicators. In an example, a method for adjusting and/or controlling the operation of an implanted medical device in a person can comprise: using machine learning and/or artificial intelligence to analyze data which includes the operating parameters of the implanted medical device over time and the values of one or more biometric indicators of that person over time in order to identify the size and lag time of the effects of changes in those parameters on changes in those biometric indicators.

In an example, a method for moving biometric indicators in a desired direction and/or into a desired range of values can comprise receiving data concerning operating parameters of an implanted medical device during a first time period; receiving data concerning the values of selected biometric indicators during the first time period; using machine learning and/or artificial intelligence to identify the effects of the operating parameters on the selected biometric indicators; receiving data concerning the values of the selected biometric indicators during a second period; using machine learning and/or artificial intelligence to identify whether one or more of the biometric indicators should be changed; and using machine learning and/or artificial intelligence and the previously identified effects to determine how the operating parameters of the implanted medical device should be changed to change the one or more biometric indicators in a desired manner.

In an example, an artificial blood flow lumen and/or vessel can be implanted into fluid communication with a natural blood vessel by one or more connecting members or connection methods selected from the group consisting of: endovascular and/or transluminal insertion and expansion, surgical anastomosis, surgical sutures, purse string suture, drawstring, pull tie, friction fit, surgical staples, tissue adhesive, gel, fluid seal, chemical bonding, cauterization, blood vessel connector and/or joint, vessel branch, twist connector, helical threads or screw connector, connection port, interlocking joints, tongue and groove connection, flanged connector, beveled ridge, magnetic connection, plug connector, circumferential ring, inflatable ring, and snap connector.

In an example, artificial intelligence (AI) can be trained on historical data on the operating parameters of an implanted medical device and the values of biometric indicators, wherein the AI is trained to identify which set, patterns, configurations, and/or values of the operating parameters are associated with changing the biometric indicators in an less healthy manner and which sets, patterns, configurations, and/or values of the operating parameters are associated with changing the biometric indicators in a healthy manner. In an example, a method can use machine learning and/or artificial intelligence to identify the effects of the operation of an implanted medical device on selected biometric indicators, taking into account a person's demographic information and clinical history.

In an example, machine learning and/or AI for adjusting operating parameters of an implanted medical device can employ linear discriminant analysis. In an example, machine learning and/or AI for adjusting operating parameters of an implanted medical device can employ a support vector machine. In an example, machine learning and/or AI for adjusting operating parameters of an implanted medical device can employ Fourier transformation. In an example, machine learning and/or AI for adjusting operating parameters of an implanted medical device can employ data mining.

In an example, a system can include one or more sensors components selected from the group consisting of: neural impulse sensor, ballistocardiographic sensor, chemoreceptor, electromagnetic impedance sensor, humidity sensor, photodiode, strain gauge, ambient humidity sensor, carbon dioxide level, electrochemical sensor, heart rate monitor, olfactory sensor, ambient noise sensor, cardiopulmonary function sensor, electrogoniometer, hemoencephalography (HEG) sensor, optoelectronic sensor, brain-to-computer interface (BCI), and dial.

In an example, a system can adjust parameters of cardiac functioning in response to sleep (and/or stage of sleep) as detected by a wearable electromagnetic brain activity sensor. In an example, a system can adjust parameters of cardiac functioning in response to relaxation level as detected by a wearable electromagnetic brain activity sensor. In an example, a system can adjust parameters of cardiac functioning in response to level of mental exertion or focus as detected by a wearable electromagnetic brain activity sensor. In an example, a system can adjust parameters of cardiac functioning in response to high carbon dioxide levels detected by a wearable electromagnetic brain activity sensor. In an example, a system can adjust parameters of cardiac functioning in response to abnormal values of one or more of these biometric indicators or conditions as detected by a wearable electromagnetic brain activity sensor.

In an example, an electromagnetic brain activity sensor can be configured to collect data concerning electromagnetic brain activity which is affected by physical activity and/or exercise level. Physical activity and/or exercise level can trigger changes in electromagnetic brain activity. In an example, a system can adjust parameters of cardiac functioning in response to physical activity and/or exercise level as detected by a wearable electromagnetic brain activity sensor.

In an example, a system can include one or more wearable sensors selected from the group consisting of electromagnetic energy sensor, bioimpedance sensor, skin and tissue impedance sensor, resistance sensor, galvanic skin response (GSR) sensor, ECG sensor, EEG sensor, and electrochemical sensor. In an example, an electromagnetic energy sensor can be an electroencephalographic (EEG) sensor. In an example, an electromagnetic energy sensor can be an electromagnetic brain activity sensor. In an example, an electromagnetic energy sensor can be a brain activity sensor which collects data concerning the natural emission of electromagnetic energy by a person's brain. In an example, an electromagnetic energy sensor can be a wearable electromagnetic brain activity sensor and/or wearable electroencephalographic (EEG) sensor.

In an example, one or more electromagnetic energy sensors can be selected from the group consisting of: action potential sensor, bipolar electrode, capacitive electrode, capacitive sensor, conductance electrode, conductance sensor, dry electrode, wet electrode, electrical resistance sensor, electrocardiographic (ECG) sensor, electrode, electroencephalographic (EEG) sensor, electromagnetic brain activity sensor, electromagnetic path, electromagnetic sensor, electromyographic (EMG) sensor, galvanic skin response (GSK) sensor, impedance sensor, inductance sensor, interferometer, magnometer, neural action potential sensor, neural impulse sensor, and piezoelectric sensor.

In an example, there can be a normal range (including minimum and maximum) for a biometric indicator. In an example, a biometric indicator which is measured and monitored in a system can be Pulsatile Blood Volume (PBV). In an example, a biometric indicator which is measured and monitored by a system can be sleep status or stage. In an example, a biometric indicator which is measured and managed by a system can be the level of carbon dioxide in a person's body tissue and/or fluid. In an example, a biometric indicator which is measured and managed by a system can be body hydration level. In an example, a biometric indicator which is measured and managed by a system can be Pulsatile Blood Lag (PBL).

In an example, a system or method can adjust a person's cardiac functioning based on the person's peripherally-detected heart rate and/or changes in the peripherally-detected heart rate. In an example, a system or method can adjust a person's cardiac function based on their whole-body posture and/or configuration. In an example, a system or method can adjust a person's cardiac function based on their tissue impedance level. In an example, a system or method an adjust a person's cardiac function based on their skin moisture level. In an example, a system or method an adjust a person's cardiac function based on identification of a specific whole-body posture and/or configuration.

In an example, a system or method can comprise using machine learning and/or artificial intelligence to analyze one or more biometric indicators and, in response, adjust the operating parameters of an implantable cardiac rhythm management device. In an example, a method can comprise using machine learning and/or artificial intelligence to analyze one or more biometric indicators and, in response, adjust the operating parameters of a cardiac stimulation device. In an example, a method can comprise using machine learning and/or artificial intelligence to analyze one or more biometric indicators and, in response, adjust the operating parameters of a heart failure device.

In an example, a closed loop system or method for human circulatory assistance can increase blood circulation by adjusting the operation of a cardiac pacemaker in response to a high carbon dioxide level in a person's body tissue and/or fluid. In an example, a closed loop system can increase blood circulation by adjusting the operation of a cardiac pacemaker in response to abnormal Pulsatile Blood Volume (PBV) or variation thereof. In an example, a closed loop system can increase blood circulation by adjusting the operation of a cardiac pacemaker in response to abnormal Pulsatile Blood Lag (PBL).

In an example, a system can comprise a wearable sensor embodied in a hat, baseball cap, skull cap, or hair comb. In an example, a system can comprise a wearable sensor embodied in a pair of shorts or pants. In an example, a system can comprise an ear ring which is attached to a person's earlobe by magnetic attraction of members on opposite sides of the earlobe. In an example, a system can comprise an ear ring which is attached to a person's earlobe by pressure (e.g. a clamp or clip). In an example, a system can comprise a wearable sensor embodied in a sock, shoe, leg band, knee brace, pair of pants, underpants, jumpsuit, or pair of shorts.

In an example, a wearable component of a system can be a wrist and/or arm band. In this example, a wearable device can be a wrist-worn device such as a smart watch. In an example, a wearable component of a system can be selected from the group consisting of an armlet, bangle, bracelet, cuff, fitness band, gauntlet, sleeve, smart watch, strap, watch, and wrist band. In an example, a wearable component of a system can be an ear ring, ear insert, or other ear-worn device. In an example, a wearable device of a system can be worn on a person's ear or inserted into a person's ear canal.

In an example, a wearable device of a system can be worn on a person's wrist or forearm. In an example, a biometric sensor can be located in a secondary housing of a wrist-worn device, wherein the secondary housing is worn on the ventral side of the wrist. In an example, a wearable device can be selected from the group consisting of: finger ring, wrist watch (housing, band, or both), wrist band (e.g. fitness band), pin, and earlobe clip. In an example, a wearable component of a system can have two flexible straps, bands, sides, or ends which are placed around a person's wrist and/or arm and then removably-fastened together around the wrist and/or arm by an attachment mechanism selected from the group consisting of: buckle, button, clasp, clip, hook, hook-and-eye mechanism, magnet, pin, plug, prong, and snap.

In an example, a wearable device of a system can be worn on a person's upper arm. In an example, a wearable device of a system can be worn on a person's torso. In an example, a wearable device of a system can be worn on a person's leg. In an example, a wearable device of a system can be worn on a person's head. In an example, a wearable component of a system can be worn on a person's wrist and/or forearm. In an example, a wearable component of a system can be worn on a person's finger and/or hand. In an example, a wearable component of a system can be worn on a person's elbow, upper arm, and/or shoulder. In an example, a wearable component of a system can be worn on, over, and/or near one or both of a person's eyes. In an example, a wearable component of a system can be worn on, around, or within a person's ear.

In an example, a wearable device of a system can be a finger sleeve made from elastic fabric with embedded biometric sensors. In an example, a wearable device of a system can be a finger ring with embedded biometric sensors. In an example, the wearable component can be inserted (partially or fully) into the ear canal, attached to the earlobe, worn around a portion of the outer ear, or a combination thereof. In an example, an ear-worn wearable component of a system can also include a prong, arm, or other protrusion which extends forward onto the person's temple and/or their forehead.

In an example, a sensor to collect data on a biometric indicator can be located on the ventral and/or distal side of an ear lobe. In an example, a sensor to collect data on a biometric indicator can be located on the dorsal and/or proximal side of an ear lobe. In an example, sensors to collect data on a biometric indicator can be located on the inner (e.g. closest to body) surface of a finger ring or finger sleeve. In an example, movement of an emitter, a receiver, or both along a track, channel, or slot can enable customization of a device to the anatomy of a specific person for more accurate measurement of that person's biometric indicator value. In an example, movement of an emitter, a receiver, or both by a rotating member can enable more accurate measurement of a biometric indicator value in the body. In an example, such movement of an emitter, a receiver, or both can enable customization of a device to the anatomy of a specific person for more accurate measurement of that person's biometric indicator value.

In an example, a person's cardiac functioning can be adjusted based on detected troponin level and/or changes in troponin level. In an example, a person's cardiac functioning can be adjusted based on detected lactate (and/or lactic acid) levels (or changes in those levels). In an example, a person's cardiac functioning can be adjusted based on detected glucose levels and/or changes in glucose levels. In an example, a person's cardiac functioning can be adjusted based on detected electrolyte levels and/or changes in electrolyte levels. In an example, a person's cardiac functioning can be adjusted based on detected carbon dioxide levels and/or changes in carbon dioxide levels. In an example, a person's cardiac functioning can be adjusted based on detected body pH level and/or changes in body pH level.

In an example, a system can increase (or decrease) the frequency of a person's heart beats and/or the magnitude of a person's heart contractions in response to a change in the person's whole-body posture and/or configuration as detected by one or more wearable biometric sensors. In an example, a system can adjust a person's cardiac function based on identification of a specific type of activity based on measured whole-body posture and/or configuration. In an example, a person's whole-body posture and/or configuration can be measured by one or more motion sensors, electromyographic (EMG sensors), and/or bend sensors. One or more motion sensors can be selected from the group consisting of: accelerometer, gyroscope, inclinometer, strain sensor, stretch sensor, and electrogoniometer. In an example, a method can comprise receiving biometric data from a motion sensor, accelerometer, patient movement sensor, gyroscope, and/or train gauge.

In an example, a system or method can adjust a person's cardiac function based on their whole-body posture and/or configuration. In an example, a person's whole-body posture and/or configuration can be measured by one or more motion sensors, electromyographic (EMG sensors), and/or strain/stretch sensors. In an example, a motion sensor can be an inertial-based motion sensor, a strain/stretch-based motion sensor, or a combination of both. In an example, a wearable photoplethysmography (PPG) device can further comprise an inertial motion sensor (such as an accelerometer and/or a gyroscope). In an example a body motion and/or configuration sensor can be selected from the group consisting of: accelerometer, altimeter, electrogoniometer, electrogoniometer, electromyographic (EMG) sensor, GPS sensor, gyroscope, inclinometer, motion sensor, pressure sensor, stretch sensor, and strain gauge. A plurality of electromyographic (EMG) sensors can provide more accurate measurement of whole-body activity level than a similarly-placed plurality of motion sensors because electromyographic sensors can measure isometric exertion.

In an example, a system or method can optimize cardiac function for stress level. In an example, a system or method can optimize cardiac function for sleep and/or for different stages of sleep. In an example, a system or method can optimize cardiac function for relaxation level. In an example, a system or method can optimize cardiac function for level of mental exertion or focus. In an example, a system or method can adjust parameters of cardiac functioning in response to high lactate (and/or lactic acid) levels detected by an electromagnetic muscle activity sensor. In an example, a system or method can adjust parameters of cardiac functioning in response to a variation in whole-body activity level which is detected by an electromagnetic muscle activity sensor or array of electromagnetic muscle activity sensors. In an example, a system or method can adjust parameters of cardiac functioning in response to a high or low whole-body activity level detected by one or more electromagnetic muscle activity sensors (such as EMG sensors). In an example, automatic adjustment of cardiac functioning in response to detection of abnormal biometric indicator values can help to restore underlying biological and/or physiological processes to their proper functioning.

In an example, sensors to measure a biometric indicator concerning a person's body can be a pattern of optically-transmissive pathways which are printed onto an article of clothing or clothing accessory using optically-transmissive ink. In an example, sensors to measure a biometric indicator concerning a person's body can be a pattern of electromagnetic pathways which is printed onto an article of clothing or clothing accessory using electroconductive ink. In an example, an alternating sequence of electromagnetic energy emitters and receivers can be distributed around the circumference an ankle band or smart sock. In an example, an alternating sequence of electromagnetic energy emitters and receivers can be distributed around (at least half of) the circumference of a finger ring or finger sleeve.

In an example, sensors to measure a biometric indicator can be formed by a plurality of optically-transmissive threads, yarns, fibers, or layers in an article of clothing or clothing accessory. In an example, sensors to measure a biometric indicator can be formed by a plurality of electroconductive threads, yarns, fibers, or layers in an article of clothing or clothing accessory. In an example, sensors to measure a biometric indicator concerning a person's body can be formed by a grid or matrix of electroconductive threads, yarns, fibers, or layers in an article of clothing or clothing accessory. In an example, sensors to measure a biometric indicator concerning a person's body can be formed by a grid or matrix of optically-transmissive threads, yarns, fibers, or layers in an article of clothing or clothing accessory. In an example, sensors to measure a biometric indicator concerning a person's body can be a woven grid or matrix of optically-transmissive threads, yarns, fibers, or layers in an article of clothing or clothing accessory. In an example, sensors to measure a biometric indicator concerning a person's body can be a woven grid or matrix of electroconductive threads, yarns, fibers, or layers in an article of clothing or clothing accessory.

In an example, a wearable device can be temporarily and removably adhered to a person's skin. In an example, a wearable device can be an electronically-functional tattoo with biometric sensors. In an example, a wearable device can be a temporary smart tattoo with biometric sensors. In an example, a wearable device can be a smart watch with embedded biometric sensors. In an example, a wearable device can be a smart adhesive patch and/or an electronically-functional adhesive patch with biometric sensors. In an example, a wearable device can be a permanent smart tattoo and/or a permanent electronically-functional tattoo with embedded biometric sensors.

In an example, a system or method can include receiving data from one or more biochemical and/or biologic sensors selected from the group consisting of: DNA-based sensor, electrochemical sensor, electronic nose, electroosmotic sensor, electrophoresis sensor, electroporation sensor, enzyme-based sensor, fat sensor, glucose sensor, HDL sensor, LDL sensor, membrane sensor, micronutrient sensor, microorganism-based sensor, multiple-analyte sensor array, nucleic acid-based sensor, olfactory sensor, osmolality sensor, pH level sensor, plurality of cross-reactive sensors, protein-based sensor, reagent-based sensor, receptor-based sensor, RNA-based sensor, saturated fat sensor, sodium sensor, and trans fat sensor.

In an example, a biometric sensor can be attached to the sidepiece (e.g. the temple) of eyewear. In an example, a biometric sensor of a system can be integrated into a sock, shoe, leg band, knee brace, pair of pants, underpants, jumpsuit, or pair of shorts. In an example, a biometric sensor of a system can be integrated into a baseball cap, face mask, hair band, hair clip, hair comb, hair pin, hat, headband, head-encircling EEG sensor band, headphones, headset, helmet, nose plug, nose ring, respiratory mask, skull cap, tiara, or visor. In an example, a biometric sensor of a system can be integrated into a finger ring, finger sleeve, artificial finger nail, finger nail attachment, finger tip (thimble), or glove. In an example, biometric sensors can be distributed around (at least half of) the circumference of an ankle band or smart sock. In an example, biometric sensors can be attached to, embedded into, woven into, sewn into, or printed onto an article of clothing or clothing accessory.

In an example, a wearable device can be selected from the group consisting of a smart watch or watch band, a wrist or arm band, a finger ring, a sleeve, an ear bud or other ear insert, a chest strap, a smart sock, an adhesive patch, and smart glasses. In an example, a wearable device of a system can be eyeglasses with embedded biometric sensors. In an example, a wearable component of a system can be selected from the group consisting of: Augmented Reality (AR) eyewear, contact lens, electronically-functional eyewear, eyeglasses, goggles, monocle, and Virtual Reality (VR) eyewear. In an example, a wearable device of a system can be an article of clothing or clothing accessory with biometric sensors.

In an example, a wearable device can have a form which is selected from the group consisting of: smart finger ring, smart watch housing and/or band, fitness band, upper arm band, ankle band, smart sock, smart eyeglasses, smart contact lens, smart ear ring, and ear bud. In an example, a wearable device can be an ear ring, earlobe clip, ear bud, ear plug, hearing aid, or ear-worn speaker/microphone with embedded electromagnetic energy sensors. In an example, a wearable component of a system can be a hearable device. In an example, a wearable device can be an arm band or elbow sleeve with embedded biometric sensors. In an example, a wearable device can be a headband, an intra-oral appliance, or a nose ring. In an example, a wearable device can be a finger ring, smart watch, wrist band, ear ring, earlobe clip, ankle band, or smart sock. In an example, a wearable device can be a contact lens with embedded optical or electromagnetic energy sensors to measure a biometric indicator.

In an example, data concerning one or more biometric indicators can be obtained from sensors on eyewear (e.g. eyeglasses) which is worn by the person. In an example, data concerning one or more biometric indicators can be obtained from sensors on an elastic band which is worn by the person. In an example, data concerning one or more biometric indicators can be obtained from sensors on an earbud or hearing aid which is worn by the person. In an example, data concerning one or more biometric indicators can be obtained from sensors on an adhesive patch which is worn by the person. In an example, data concerning one or more biometric indicators can be obtained from sensors on a smart collar which is worn by the person. In an example, data concerning one or more biometric indicators can be obtained from sensors on a shirt (e.g. smart shirt with imbedded sensors) which is worn by the person.

In an example, a system can further comprise one or more components selected from the group consisting of: barometric pressure sensor, chewing sensor, electromyographic (EMG) sensor, hydration sensor, photoplethysmography (PPG) sensor, stretch sensor, allergen sensor, capacitive sensor, electrical resistance sensor, gyroscope, multi-axial accelerometer, drug reservoir, battery, cholesterol sensor, electronic tablet, hygrometry sensor, piezoelectric sensor, swallow sensor, bend sensor, chromatographic sensor, and electronically-functional eyewear. In an example, one or more light emitters of a wearable photoplethysmography (PPG) device can be of a type which is selected from the group consisting of light emitting diode (LED), laser diode (LD), micro-plasma emitter, and incandescent bulb. In an example, one or more light receivers of a wearable photoplethysmography (PPG) device can be selected from the group consisting of photodetector, optical detector, photodiode, and phototransistor.

In an example, a wearable photoplethysmography (PPG) device can include one or more light barriers between the environment and a light receiver to block transmission of ambient light to the light receiver. In an example, a wearable photoplethysmography (PPG) device can include an inflatable member or an electromagnetic actuator which holds a light emitter, a light receiver, and/or a light guide snugly against a person's body to reduce errors due to body motion. In an example, a wearable photoplethysmography (PPG) device can include a spring or elastic member whose tension and/or length is adjusted to hold a light emitter, a light receiver, and/or a light guide snugly against a person's body. In an example, a wearable photoplethysmography (PPG) device can also include compressible foam which holds a light emitter, a light receiver, and/or a light guide snugly against a person's body

In an example, a wearable component of a system can be an ankle band or a smart sock with embedded biometric sensors. In an example, an alternating sequence of light emitters and receivers can be distributed around the circumference an ankle band or smart sock. In an example, data concerning one or more biometric indicators can be obtained from sensors on a sock (e.g. smart sock with imbedded sensors) which is worn by the person. In an example, the wearable device can be selected from the group consisting of a smart watch or watch band, a wrist or arm band, a finger ring, a sleeve, an ear bud or other ear insert, a chest strap, a smart sock, an adhesive patch, and smart glasses. In an example, a wearable photoplethysmography (PPG) device can be embodied in a wearable device with a form which is selected from the group consisting of earbud, hearing aid, ear ring, headset, headphone, nose ring, eyeglasses, necklace, collar, finger ring, wrist band, smart watch, smart cuff, bracelet, arm band, sleeve, toe ring, sock, ankle band, and belt.

In an example, a wearable device of a system can have a form which is selected from the group consisting of: headphones or headset, chest strap, contact lens, finger sleeve, hearing aid, Virtual Reality (VR) eyewear, ear plug or buds, and helmet. In an example, a biometric sensor of a system or method can be worn on and/or around a person's chest or waist. In an example, data concerning one or more biometric indicators can be obtained from sensors on a chest strap which is worn by the person. In an example, a wearable device can be a leg band with embedded biometric sensors. In an example, a light emitter can emit light from the inward side of a wearable device toward the surface of a person's body (e.g. finger, wrist, arm, ear, or leg).

In an example, this device can further comprise one or more computer-to-human interface (HCI) components. One or more computer-to-human interface components can be selected from the group consisting of: display screen, light emitter and/or light-emitting array, light-emitting fabric, optical emitter, speaker, buzzer, or other sound-emitting member, electromagnetic signal generator, vibrating member, actuator, Micro Electro Mechanical Systems (MEMS), augmented reality eyewear, virtual reality eyewear, and electronically-functional eyewear.

In an example, a system can automatically vary the distance between a light emitter and a light receiver to scan through a range of tissue depths, locations, and/or types in order to obtain more accurate measurement of a biometric indicator value in the body. In an example, the depth, breadth, location, and/or type of body tissue or fluid from which light from a light emitter is reflected and received by a light receiver can be changed by adjusting the distance between a light emitter and a light receiver. In an example, the distance between a light emitter and a light receiver can be adjusted in order to more accurately measure a biometric indicator value in the body. In an example, the distance between a light emitter and a light receiver can be adjusted automatically (in an iterative manner) by a system in order to more accurately measure a biometric indicator value in the body for a specific person, for a specific type of activity, or for a specific configuration of the system relative to the person's body surface.

In an example, a system can comprise one or more light emitters which deliver light to body tissue and/or fluid via direct optical communication. In an example, a light emitter and light receiver can be paired together. In an example, a light emitter and a light receiver can be on the same circumferential line (e.g. circle) of a wearable device, but at different radial locations around this circumference. In an example, emission and reception of light between different pairs of light emitters and light receivers can be multiplexed. In an example, the distance between a light emitter and a light receiver can be adjusted automatically (in an iterative manner) by a device in order to more accurately measure a biometric indicator value in the body for a specific person, for a specific type of activity, or for a specific configuration of the device relative to the person's body surface. In an example, a light emitter can emit coherent or polarized light.

In an example, a system can further comprise one or more prisms which guide light from a light emitter toward body tissue and/or fluid at a selected angle or location. In an example, a system can further comprise one or more prisms which guide light from body tissue and/or fluid to a light receiver. In an example, a wearable device can comprise a prism and/or filter which splits ambient light into red light and green light, wherein the red light is in optical communication with a first light receiver (after the red light has interacted with body tissue and/or fluid) and the green light is in optical communication with a second light receiver (after the green light has interacted with body tissue and/or fluid).

In an example, a wearable sensor component of a system can include an array, grid, and/or matrix of (alternating) light emitters and receivers. In an example, the depths, breadths, locations, and/or types of body tissue or fluid from which light beams from a plurality of light emitters are reflected can be determined by a selected geometric configuration of the plurality of light emitters and a light receiver. In an example, a plurality of light emitters can be configured in a circular array in proximity to a light receiver. In an example, a plurality of light receivers can be configured in a polygonal array including a light emitter.

In an example, a system can automatically vary the angle of light from a light emitter to scan through a range of tissue depths, locations, and/or types in order to obtain more accurate measurement of a biometric indicator value in the body. In an example, a device can automatically vary the power and/or intensity of light from a light emitter to scan through a range of tissue depths, locations, and/or types in order to obtain more accurate measurement of a biometric indicator value in the body. In an example, a device can automatically vary the geometric configuration of a light emitter and a plurality of light receivers in order to scan through a range of tissue depths, locations, and/or types in order to measure a biometric indicator value in the body more accurately.

In an example, a system can include one or more light guides which direct light energy from a first location, angle, and/or transmission vector to a second location, angle, and/or transmission vector. In an example, a first light receiver can receive light from a first (radial) location relative to a body member and a second light receiver can receive light from a second (radial) location relative to the body member which is different than the first location. In an example, the first light receiver can have a first location relative to the person's body and the second light receiver can have a second location relative to the person's body. In an example, the first light emitter can have a first location relative to the person's body and the second light emitter can have a second location relative to the person's body. In an example, a first subset of light emitters can direct light beams toward a first location on a person's body and a second subset of light emitters can direct light beams toward a second location on the person's body.

In an example, different sets of light emitters and receivers can have different locations wherein light is transmitted through or reflected by a person's body. In an example, a system can automatically vary the geometric configuration of a plurality of light emitters and a light receiver in order to scan through a range of tissue depths, locations, and/or types in order to measure a biometric indicator value in the body more accurately. In an example, a device can automatically vary the geometric configuration of a plurality of light emitters and a light receiver in order to scan through a range of tissue depths, locations, and/or types in order to measure a biometric indicator value in the body more accurately.

In an example, the depths, breadths, locations, and/or types of body tissue or fluid from which light beams from a plurality of light emitters are reflected can be determined by a selected geometric configuration of the plurality of light emitters and a light receiver. In an example, the depths, breadths, locations, and/or types of body tissue or fluid from which light beams are reflected and received by a plurality of light receivers can be determined by a selected geometric configuration of a light emitter and the plurality of light receivers. In an example, light beam emission and reception between different associated pairs of light emitters and receivers can be sequenced to isolate measurement of biometric values from different tissue depths and/or locations.

In an example, this device can further comprise one or more environmental and/or ambient sensors selected from the group consisting of: air-borne allergen sensor, air-borne pollution sensor, air-borne toxin sensor, altitude sensor, ambient air composition sensor, ambient air quality sensor, ambient carbon dioxide sensor, ambient carbon monoxide sensor, ambient electromagnetic radiation sensor, ambient humidity sensor, ambient light sensor, ambient noise sensor, ambient oxygen sensor, ambient sound pattern recognition sensor, ambient temperature sensor, ambient ultrasonic energy sensor, ambient voices sensor, and barometric pressure sensor.

In an example, a wearable component of a system can be an ear ring, earlobe clip, ear bud, ear plug, hearing aid, or ear-worn speaker/microphone with embedded biometric sensors. In an example, a wearable component of a system can be selected from the group consisting of: ear bud, ear hook, ear plug, ear ring, earlobe clip, earphone, earpiece, earring, ear-worn Bluetooth communication device, electroencephalographic (EEG) sensor, oximeter, headphone, headset, and hearing aid. In an example, a spectroscopic sensor of a system can be configured to receive light energy which has been reflected from, or passed through, aqueous humour, blood, blood vessels, body fat, brain tissue, dermis, ear drum, earlobe, epidermis, fat tissue, intercellular fluid, lung tissue, lymphatic fluid, lymphatic passageways, muscle tissue, nerve tissue, saliva, skin, sweat, and/or tears in order to monitor lactate (and/or lactic acid) levels (or changes in those levels).

In an example, a spectroscopic sensor of a system can be configured to receive light energy which has been reflected from, or passed through, blood in order to monitor blood or tissue oxygen levels (or changes in those levels). In an example, a spectroscopic sensor of a system can be configured to receive light energy which has been reflected from, or passed through, body tissue, organs, and/or fluid selected from the group consisting of: aqueous humour, blood, blood vessels, body fat, brain tissue, dermis, ear drum, earlobe, epidermis, fat tissue, intercellular fluid, lung tissue, lymphatic fluid, lymphatic passageways, muscle tissue, nerve tissue, saliva, skin, sweat, and tears. In an example, a spectroscopic sensor of a system can detect the compositions of blood, sweat, and tears.

In an example, a spectroscopic sensor of a system can be configured to receive light energy which has been reflected from, or passed through, aqueous humour, blood, blood vessels, body fat, brain tissue, dermis, ear drum, earlobe, epidermis, fat tissue, intercellular fluid, lung tissue, lymphatic fluid, lymphatic passageways, muscle tissue, nerve tissue, blood, saliva, skin, sweat, and/or tears in order to monitor glucose levels and/or changes in glucose levels. In an example, a spectroscopic sensor of a system can be configured to receive light energy which has been reflected from, or passed through, aqueous humour, blood, blood vessels, body fat, brain tissue, dermis, ear drum, earlobe, epidermis, fat tissue, intercellular fluid, lung tissue, lymphatic fluid, lymphatic passageways, muscle tissue, nerve tissue, saliva, skin, sweat, and/or tears in order to monitor oxygen levels (or changes in those levels).

In an example, a biometric light energy sensor of a system (such as a spectroscopic sensor) can be configured to collect data concerning one or more biometric indicators or conditions selected from the group consisting of: albumin level, anaerobic threshold, atrial fibrillation, bilirubin level, blood carbon dioxide level, blood flow, blood glucose level, blood hydration, blood or tissue oxygen (SpO2), blood pH level, blood pressure, blood pulsation, blood urea nitrogen (BUN), blood vessel dilation, blood volume, body acceleration, body balance, body configuration, body fat density, body hydration, body motion, whole-body posture, body speed, bradycardia, brainwave activity, brainwave frequency band levels, breathing rate, calcium level, caloric intake, caloric metabolism, carbon dioxide level, carbon monoxide level, carboxyhemoglobin level, cardiac output, cardiopulmonary function, chemical composition of blood, chemical composition of intercellular fluid, chemical composition of sweat, chemical composition of tears, chloride level, cholesterol (HDL) level, cholesterol (LDL) level, copper level, creatine level, creatine phosphokinase level, digestive system functioning, eating behavior, electrocardiographic (ECG) patterns, electroencephalographic (EEG) patterns, electrolyte levels, electromagnetic brain activity, electromagnetic energy from the body, electromagnetic evoked potentials of the brain, and electromyographic (EMG) patterns.

In an example, a spectroscopic sensor of a system can be configured to collect data concerning light energy reflected from, or having passed through, blood and/or blood vessels in order to measure one or more biometric indicators or conditions selected from the group consisting of: cholesterol (LDL) level, copper level, creatine kinase level, creatine level, creatine phosphokinase level, electrolyte levels, glucose level, heart rate, heart rate variability (HRV), hemoglobin level, hormone level, hydration, hypertension, iron level, lactate level, lactic acid level, lipid levels, magnesium level, methemoglobin level, myoglobin level, nickel level, nitrogen level, oxygen level, oxygen saturation, partial pressure of carbon dioxide, partial pressure of oxygen, pH level, phosphorus level, potassium level, protein levels, pulse, sodium level, thyroid stimulating hormone (TSH) level, triglyceride level, troponin level, and urea nitrogen level.

In an example, a wearable device of a system can include one or more spectroscopic sensors selected from the group consisting of: ambient light spectroscopic sensor, analytical chromatographic sensor, backscattering spectrometry sensor, spectroscopic camera, chemiluminescence sensor, chromatographic sensor, coherent light spectroscopic sensor, colorimetric sensor, fiber optic spectroscopic sensor, fluorescence sensor, gas chromatography sensor, infrared light sensor, infrared spectroscopic sensor, ion mobility spectroscopic sensor, laser spectroscopic sensor, liquid chromatography sensor, mass spectrometry sensor, near infrared spectroscopic sensor, optoelectronic sensor, photocell, photochemical sensor, Raman spectroscopy sensor, spectral analysis sensor, spectrographic sensor, spectrometric sensor, spectrometry sensor, spectrophotometer, spectroscopic glucose sensor, spectroscopic oximeter, ultraviolet light sensor, ultraviolet spectroscopic sensor, variable focal-length camera, video camera, visible light spectroscopic sensor, and white light spectroscopic sensor.

In an example, a wearable component of a system can be worn in a manner similar to an ear bud, ear hook, ear plug, ear ring, earlobe clip, earphone, earpiece, earring, ear-worn Bluetooth communication device, electroencephalographic (EEG) sensor, oximeter, headphone, headset, or hearing aid. In an example, an ear ring with embedded biometric sensors can be attached to a person's earlobe through a pierced opening in the ear lobe. In an example, a wearable photoplethysmography (PPG) device can be worn on a person's body at location which is selected from the group consisting of a person's ear (e.g. ear lobe or ear canal), nose (e.g. septum), forehead, neck, hand (e.g. finger), wrist, arm, toe, ankle, leg, and waist. In an example, a circumferential array of sensors can be distributed around an ear bud or ear plug which is inserted into a person's ear canal. In an example, a spectroscopic sensor of a system can be configured to receive light energy which has been reflected from, or passed through, aqueous humour, blood, blood vessels, body fat, brain tissue, dermis, ear drum, earlobe, epidermis, fat tissue, intercellular fluid, lung tissue, lymphatic fluid, lymphatic passageways, muscle tissue, nerve tissue, blood, saliva, skin, sweat, and/or tears in order to monitor water levels and/or changes in hydration and/or water level.

In an example, a spectroscopic sensor of a system can be configured to collect data concerning light energy reflected from, or having passed through, blood and/or blood vessels in order to measure one or more biometric indicators or conditions selected from the group consisting of: albumin level, bilirubin level, blood flow, blood glucose level, blood hydration, blood or tissue oxygen (SpO2), blood pH level, blood pressure, blood pulsation, blood urea nitrogen (BUN), blood vessel dilation, blood volume, body hydration, calcium level, caloric intake, caloric metabolism, carbon dioxide level, carbon dioxide level, carbon monoxide level, carboxyhemoglobin level, chloride level, or HDL level.

In an example, data from the light receiver can be analyzed to determine how the spectrum of directed light has been changed by reflection from, or passage through, the person's body tissue and/or fluid. In an example, a light receiver which receives ambient light after that light has interacted with body tissue and/or fluid can be referred to as a spectroscopy sensor. In an example, a plurality of light emitters can be configured in a linear array in proximity to a light receiver. In an example, a light receiver can be automatically tilted, rotated, raised, or lowered if the wearable housing which holds it moves relative to the body surface on which it is worn. In example, differences in the spectrum of light emitted from a light emitter and the spectrum of light received by a light receiver can be analyzed to measure tissue (and/or blood) oxygen levels.

In an example, a light receiver can receive light with a first light wavelength (or wavelength range or spectral distribution) during a first time period and can receive light with a second light wavelength (or wavelength range or spectral distribution) during a second time period. In an example, a light emitter can emit light energy with a first light wavelength (or wavelength range or spectral distribution) during a first time period and can emit light energy with a second light wavelength (or wavelength range or spectral distribution) during a second time period. In an example, a system can comprise a first light receiver which receives light energy with a first light wavelength (or wavelength range or spectral distribution) and a second light receiver which simultaneously receives light energy with a second light wavelength (or wavelength range or spectral distribution) during the same time period. In an example, blood or tissue oxygen saturation can be based on differential absorption of two different light wavelengths by blood.

In an example, changes in the amount (or concentration) of a selected analyte in body tissue can change the spectrum of light passing through and/or reflected by the body tissue. In an example, a person's skin moisture level can be measured by reflecting light energy from (and/or passing light energy through) body tissue. In an example, a person's tissue impedance level can be measured by reflecting light energy from (and/or passing light energy through) body tissue. In an example, body oxygenation can be estimated based on the ratio of changes in the spectra of light beams from the two light emitters due to those light beams having been transmitted through or reflected by body tissue and/or fluid. In an example, different spectroscopic sensors in a plurality of spectroscopic sensors can differ with respect to the body tissue depth from which light beams are reflected back to light receivers. In an example, light beam emission from different light emitters can be sequenced and/or multiplexed.

In an example, a system can include a wearable photoplethysmography (PPG) device which collects information concerning light which has been reflected from, or transmitted through, body tissue in order to measure one or more biometric indicators selected from the group consisting of heart rate (HR), heart rate Variability (HRV), Blood Volume Pulse (BVP), Inter-Beat Interval (IBI), Peak-to-Peak Interval (PPI), Blood Pulse Rate (BPR), Blood Volume Pulse (BVP), Blood Volume Amplitude (BVP), Peak Amplitude (PA), Pulse Area, Pulse Transit Time (PTT), Standard Deviation of Average Normal to Normal Beat (SDANN), Systolic Amplitude, Systolic Peak Amplitude, blood pressure, oxygen Saturation, VO2, VO2max, Hydration Level, and Respiration and/or Breathing Rate.

In an example, a spectroscopic sensor of a system can be configured to receive light energy which has been reflected from, or passed through, aqueous humour, blood, blood vessels, body fat, brain tissue, dermis, ear drum, earlobe, epidermis, fat tissue, intercellular fluid, lung tissue, lymphatic fluid, lymphatic passageways, muscle tissue, nerve tissue, blood, saliva, skin, sweat, and/or tears in order to monitor body temperature and/or changes in body temperature. In an example, a spectroscopic sensor of a system can be configured to receive light energy which has been reflected from, or passed through, aqueous humour, blood, blood vessels, body fat, brain tissue, dermis, ear drum, earlobe, epidermis, fat tissue, intercellular fluid, lung tissue, lymphatic fluid, lymphatic passageways, muscle tissue, nerve tissue, blood, saliva, skin, sweat, and/or tears in order to monitor electrolyte levels and/or changes in electrolyte levels.

In an example, a spectroscopic sensor of a system can be configured to receive light energy which has been reflected from, or passed through, aqueous humour, blood, blood vessels, body fat, brain tissue, dermis, ear drum, earlobe, epidermis, fat tissue, intercellular fluid, lung tissue, lymphatic fluid, lymphatic passageways, muscle tissue, nerve tissue, blood, saliva, skin, sweat, and/or tears in order to monitor hormone levels and/or changes in hormone levels. In an example, a spectroscopic sensor of a system can be configured to receive light energy which has been reflected from, or passed through, aqueous humour, blood, blood vessels, body fat, brain tissue, dermis, ear drum, earlobe, epidermis, fat tissue, intercellular fluid, lung tissue, lymphatic fluid, lymphatic passageways, muscle tissue, nerve tissue, blood, saliva, skin, sweat, and/or tears in order to monitor pH levels and/or changes in pH levels.

In an example, a system or method can comprise one or more sensors selected from the group consisting of: accelerometer, acoustic energy sensor, action potential sensor, activity level sensor, auscultatory sensor, ballistocardiographic sensor, bend sensor, biochemical sensor, blood flow sensor, blood pressure sensor, brain activity sensor, breathing rate sensor, caloric intake monitor, capacitance hygrometry sensor, capacitive sensor, cardiac function sensor, cardiopulmonary function sensor, chemiluminescence sensor, chemoreceptor, chewing sensor, chromatographic sensor, compass, conductivity sensor, core temperature sensor, cranial pressure sensor, digital camera, electrical resistance sensor, electrocardiographic (ECG) sensor, electroencephalographic (EEG) sensor, electrogoniometer, electromagnetic energy sensor, electromyographic (EMG) sensor, electroporation sensor, enzymatic sensor, eye muscle (EOG) sensor, galvanic skin response (GSR) sensor, glucose sensor, GPS sensor, gyroscope, Hall-effect sensor, heart rate monitor, heart rate sensor, hormone sensor, humidity sensor, hydration level sensor, hygrometry sensor, impedance sensor, inclinometer, inertial sensor, infrared light (IR) sensor, infrared spectroscopy sensor, ion mobility spectroscopic sensor, lactate sensor, laser sensor, light intensity sensor, magnetic energy sensor, magnetometer, medichip, and metal oxide semiconductor sensor.

In an example, biometric sensors can be spectroscopic sensors. In an example, a wearable device can comprise a plurality of spectroscopic sensors. In an example, a first light emitter can emit light with a first light frequency, color, and/or spectrum and a second light emitter can emit light with a second light frequency, color, and/or spectrum. In an example, a light emitter can emit light with frequency and/or spectrum changes over time. In an example, a wearable device can be an ear ring, earlobe clip, ear bud, ear plug, hearing aid, or ear-worn speaker/microphone with embedded spectroscopic sensors. In an example, spectroscopic sensors in a smart adhesive patch and/or an electronically-functional adhesive patch can be used to monitor the molecular composition of a person's sweat and/or gases emitted from the person's skin.

In an example, a wearable device of a system can have an optical sensor. In an example, a wearable device for collecting data on a biometric indicator can include a single light emitter and a plurality of light receivers. In an example, a wearable device for collecting data on a biometric indicator can include a plurality of spectroscopic sensors. In an example, a wearable device can perform photoplethysmography (PPG). In an example, a wearable device of a system can have an electromagnetic energy sensor. In an example, a wearable device of a system can have a chromatographic sensor. In an example, a wearable device of a system can comprise electromagnetic sensors which are embedded in (or attached to) an article of clothing or clothing accessory. In an example, a wearable component of a system can be worn in a manner similar to an armband, compression joint sleeve, full-sleeve, or shirt. In an example, a system can include one or more wearable sensors selected from the group consisting of motion sensor, accelerometer, patient movement sensor, gyroscope, and strain gauge.

In an example, a wearable device of a system can include one or more lenses. In an example, a system can further comprise one or more optical filters or lenses which change the projection and/or body incidence angle of a light beam emitted by a light emitter. In an example, a lens can selectively refract and/or focus light transmission between body tissue and a light receiver. In an example, the beam of light emitted by a light emitter can be automatically moved by using an actuator to automatically move a lens (or light guide) through which this beam is transmitted. In an example, the beam of light emitted by a light emitter can be automatically moved by using an actuator to automatically change the focal distance of a lens (or light guide) through which this beam is transmitted. In an example, a lens can selectively refract and/or focus light transmission between ambient light and body tissue. In an example, the primary emitter and the primary receiver of this device, discussed above, can be a light emitter and a light receiver, but the device can also include a (non-light-spectrum) electromagnetic emitter and a (non-light-spectrum) electromagnetic receiver.

In an example, a spring, elastic member, inflatable member, and/or electromagnetic actuator can be adjusted in response to motion detected by a motion sensor in order to hold a light emitter, a light receiver, and/or a light guide more snugly against a person's body when the body is moving a lot. In an example, movement of a light emitter, a light receiver, or both along a track, channel, or slot can enable more accurate measurement of a biometric indicator value in the body. In an example, the coherence, polarization, and/or phase of light emitted from the light emitter can be adjusted automatically to maintain accurate measurement of a biometric indicator value in the body even if the system shifts and/or moves relative to the person's body surface. In an example, the frequency, color, and/or spectrum of a beam of light emitted from a light emitter can be changed over time to create a chronological sequence of beams of light with different frequencies, colors, and/or spectrums.

In an example, eyewear can comprise a plurality of biometric sensors on the front piece and/or nose bridge of the eyewear. In an example, eyewear can comprise a plurality of biometric sensors on the frame of the eyewear. In an example, a biometric sensor of a system can be integrated into Augmented Reality (AR) eyewear, contact lens, electronically-functional eyewear, eyeglasses, goggles, monocle, or Virtual Reality (VR) eyewear. In an example, a plurality of light emitters and receivers can be incorporated into the side pieces of eyeglasses. In an example, a plurality of spectroscopic sensors can be incorporated into the side pieces of eyeglasses. In an example, a wearable device of a system can be eyewear. In an example, a wearable component of a system can be worn in a manner similar to Augmented Reality (AR) eyewear, contact lens, electronically-functional eyewear, eyeglasses, goggles, monocle, or Virtual Reality (VR) eyewear.

In an example, reflection of light by body tissue and/or fluid changes the spectrum of that light and this change in spectrum is analyzed to get information about the composition of that body tissue and/or fluid. In an example, the angle of a beam of light emitted from a light emitter can be changed over time to create a chronological sequence of beams of light with different projection and/or body incidence angles. In an example, the angle of light emitted from the light emitter can be adjusted automatically (in an iterative manner) by a device in order to more accurately measure a biometric indicator value in the body for a specific person, for a specific type of activity, or for a specific configuration of the device relative to the person's body surface. In an example, the angle of light emitted from the light emitter can be adjusted automatically to maintain accurate measurement of a biometric indicator value in the body even if the device shifts and/or moves relative to the person's body surface.

In an example, the frequency, color, and/or spectrum of light emitted from the light emitter can be adjusted automatically (in an iterative manner) by a device in order to more accurately measure a biometric indicator value in the body for a specific person, for a specific type of activity, or for a specific configuration of the device relative to the person's body surface. In an example, the frequency, color, and/or spectrum of light emitted from the light emitter can be adjusted automatically to maintain accurate measurement of a biometric indicator value in the body even if the system shifts and/or moves relative to the person's body surface.

In an example, a system can include one or more wearable spectroscopic sensors. In an example, a spectroscopic sensor can be configured to receive light energy which has been reflected from, or passed through, aqueous humour, blood, blood vessels, body fat, brain tissue, dermis, ear drum, earlobe, epidermis, fat tissue, intercellular fluid, lung tissue, lymphatic fluid, lymphatic passageways, muscle tissue, nerve tissue, blood, saliva, skin, sweat, and/or tears in order to monitor carbon dioxide levels and/or changes in carbon dioxide levels. In an example, the depth, breadth, location, and/or type of body tissue or fluid from which light from a light emitter is reflected can be changed by adjusting the coherence, polarization, and/or phase of light emitted from the light emitter. In an example, a light receiver of a spectroscopic sensor can receive light with a first light wavelength (or wavelength range or spectral distribution) during a first time period and can receive light with a second light wavelength (or wavelength range or spectral distribution) during a second time.

In an example a system can comprise a plurality and/or array of body motion and/or configuration sensors which are selected from the group consisting of: accelerometer, altimeter, electrogoniometer, electrogoniometer, electromyographic (EMG) sensor, GPS sensor, gyroscope, inclinometer, motion sensor, pressure sensor, stretch sensor, and strain gauge. In an example, this device can comprise an array, grid, and/or matrix of light emitters which differ in one or more parameters selected from the group consisting of: location and/or distance from a light receiver; distance to body surface; light beam frequency, color, and/or spectrum; light beam coherence, polarity, and/or phase; light beam power and/or intensity; light beam projection and/or body incidence angle; light beam duration; light beam size; and light beam focal distance.

In an example, the coherence, polarization, and/or phase of light emitted from the light emitter can be adjusted automatically (in an iterative manner) by a system in order to more accurately measure a biometric indicator value in the body for a specific person, for a specific type of activity, or for a specific configuration of the system relative to the person's body surface. In an example, a wearable device can comprise an array, grid, and/or matrix of light emitters which differ in one or more parameters selected from the group consisting of: location and/or distance from a light receiver; distance to body surface; light beam frequency, color, and/or spectrum; light beam coherence, polarity, and/or phase; light beam power and/or intensity; light beam projection and/or body incidence angle; light beam duration; light beam size; and light beam focal distance. In an example, a wearable light receiver can be optically isolated from a wearable light emitter by means of a light blocking layer, coating, cladding, or component so that only ambient light reflected from, or having passed through, body tissue, organs, or fluid reaches the light receiver.

In an example, a first subset of spectroscopic sensors in a plurality of spectroscopic sensors can direct light beams from a first location on a wearable device and a second subset of spectroscopic sensors in the plurality of spectroscopic sensors can direct light beams from a second location on a wearable device. In an example, a first subset of spectroscopic sensors in a plurality of spectroscopic sensors can direct light beams toward a first location on a person's body and a second subset of spectroscopic sensors in the plurality of spectroscopic sensors can direct light beams toward a second location on the person's body. In an example, a first subset of spectroscopic sensors in a plurality of spectroscopic sensors can direct light beams toward a first side of a portion of a person's body and a second subset of spectroscopic sensors in the plurality of spectroscopic sensors can direct light beams toward a second side of the portion of the person's body.

In an example, a light emitter can emit a sequence of light pulses at different selected frequencies. In an example, the depth, breadth, location, and/or type of body tissue or fluid from which light from a light emitter is reflected can be changed by adjusting the frequency, color, and/or spectrum of light emitted from the light emitter. In an example, the frequency, color, and/or spectrum of a beam of light emitted from a light emitter can be changed in response to specific environmental conditions (e.g. temperature or humidity) and/or specific activities in which the person wearing a device is engaged (e.g. high level of movement, eating, sleeping, etc.) in order to more accurately measure a biometric indicator value in the body. In an example, a first subset of spectroscopic sensors in a plurality of spectroscopic sensors can direct light beams with a first color, frequency, and/or spectrum toward a person's body and a second subset of spectroscopic sensors in the plurality of spectroscopic sensors can direct light beams with a second color, frequency, and/or spectrum toward the person's body.

In an example, a system can automatically vary the power and/or intensity of light from a light emitter to scan through a range of tissue depths, locations, and/or types in order to obtain more accurate measurement of a biometric indicator value in the body. In an example, a system can automatically vary the geometric configuration of a light emitter and a plurality of light receivers in order to scan through a range of tissue depths, locations, and/or types in order to measure a biometric indicator value in the body more accurately. In an example, a system can automatically vary the frequency, color, and/or spectrum of light from a light emitter to scan through a range of tissue depths, locations, and/or types in order to obtain more accurate measurement of a biometric indicator value in the body. In an example, a system can automatically vary the coherence, polarization, and/or phase of light from a light emitter to scan through a range of tissue depths, locations, and/or types in order to obtain more accurate measurement of a biometric indicator value in the body.

In an example, different spectroscopic sensors in a plurality of spectroscopic sensors can differ with respect to the timing and/or synchronization of light beams directed toward a person's body. In an example, different spectroscopic sensors in a plurality of spectroscopic sensors can differ with respect to the angles at which they direct light beams toward a person's body. In an example, different spectroscopic sensors in a plurality of spectroscopic sensors can differ with respect to the power or intensity of light beams which they direct toward a person's body. In an example, different spectroscopic sensors in a plurality of spectroscopic sensors can differ with respect to the polarization or coherence of light beams which where they direct toward a person's body. In an example, different spectroscopic sensors in a plurality of spectroscopic sensors can differ with respect to being at different locations around (at least half of) the circumference of a finger, wrist, arm, ankle, or toe.

In an example, a system or method can comprise one or more sensors selected from the group consisting of: Micro Electrical Mechanical System (MEMS) sensor, microcantilever sensor, microfluidic sensor, microphone, motion sensor, muscle function monitor, near-infrared spectroscopic sensor, neural impulse monitor, neurosensor, optical detector, optical sensor, optoelectronic sensor, oximetry sensor, perspiration rate sensor, pH level sensor, photochemical sensor, photodetector, photodiode, photoelectric sensor, photoplethysmographic (PPG) sensor, piezocapacitive sensor, piezoelectric sensor, piezoresistive sensor, position sensor, pressure sensor, pulse oximetry sensor, pulse rate sensor, pyroelectric sensor, radio frequency (RF) sensor, Raman spectroscopy sensor, respiration sensor, skin conductance sensor, skin moisture sensor, skin temperature sensor, sound energy sensor, spectral analysis sensor, spectrometric sensor, spectrophotometer, spectroscopic sensor, still-frame camera, strain gauge, stretch sensor, swallowing sensor, sweat sensor, systolic blood pressure sensor, temperature sensor, thermal energy sensor, thermistor, thermocouple, thermometer, thermopile, tissue impedance sensor, ultrasonic energy sensor, ultraviolet light sensor, ultraviolet spectroscopy sensor, variable impedance sensor, variable resistance sensor, variable translucence sensor, and video camera.

In an example, a spectroscopic sensor of a system can be configured to receive light energy which has been reflected from, or passed through, a person's blood and/or blood vessels in order to monitor blood pressure and/or changes in blood pressure. In an example, a spectroscopic sensor of a system can be configured to receive light energy which has been reflected from, or passed through, a person's blood and/or blood vessels in order to monitor for tachycardia or bradycardia. In an example, a spectroscopic sensor of a system can be configured to receive light energy which has been reflected from, or passed through, a person's blood and/or blood vessels in order to monitor heart rate and/or changes in heart rate.

In an example, a sensor can comprise a plurality of light emitters and light receivers. In an example, a wearable device for collecting data on a biometric indicator can include a plurality of light emitters which emit light toward a person's body tissue and/or fluid. In an example, a first light emitter can emit light with a first light coherence, polarization, and/or phase and a second light emitter can emit light with a second light coherence, polarization, and/or phase. In an example, a first light receiver can receive light with a first light wavelength (or wavelength range or spectral distribution) and a second light receiver can simultaneously receive light with a second light wavelength (or wavelength range or spectral distribution) during the same time period in order to simultaneously measure different physiological parameters, analytes, or conditions.

In an example, a sensor can emit infrared light, near-infrared light, ultraviolet light, and visible and/or white light into a person's body. In an example, one or more light emitters can deliver infrared light energy, near infrared light energy, ultraviolet light energy, and/or visible light energy to body tissue, organs, and/or fluid. In an example, a wearable device can have a red light emitter and an infrared light emitter. In an example, a light emitter can emit ultraviolet light. In an example, a light emitter can emit infrared or near-infrared light. In an example, a wearable device of a system can have both a near-infrared spectroscopic sensor and an infrared spectroscopic sensor.

In an example, a system or method can comprise an optical (e.g. spectroscopic) sensor which receives (and analyzes) light which has been reflected by a person's body tissue. In an example, a system or method can comprise an optical (e.g. spectroscopic) sensor which receives (and analyzes) light which has been transmitted through a person's body tissue. In an example, a system or method can comprise a wearable optical (e.g. spectroscopic) sensor which receives (and analyzes) light which has been reflected by a person's body tissue. In an example, a system or method can comprise a wearable optical (e.g. spectroscopic) sensor which receives (and analyzes) light which has been transmitted through a person's body tissue.

In an example, a wearable component of a system or method can comprise receiving data from one or more paired sets of light emitters and light receivers. In an example, each paired set can be configured so that light emitted from the light receiver is received by the light receiver after the light is reflected from, or passes through, body tissue or fluid. In an example, light emitters and light receivers in a plurality of spectroscopic sensors can be pair-wise associated at opposite sides of the circumference of a portion of a person's body (such as a person's finger, wrist, arm, ankle, or leg), wherein pair-wise associated at opposite sides means that each light emitter is associated with a light receiver which located on the (diametrically) opposite side of the body portion. In an example, a circumferential array of light emitters can be even spaced or distributed, with the same pair-wise distance or number of degrees between adjacent emitters.

In an example, a wearable component of a system can include one or more light receivers which receive light energy that has been reflected from, passed through, and/or scattered by body tissue, organs, and/or fluid. In an example, a wearable component of a system can include one or more light receivers which receive light energy which was originally emitted by a wearable light emitter and then subsequently reflected from, passed through, or scattered by body tissue, organs, and/or fluid. In an example, a wearable light receiver can be optically isolated from a wearable light emitter by means of a light blocking layer, coating, cladding, or component so that only light reflected from, or having passed through, body tissue, organs, or fluid reaches the light receiver. In an example, analysis of changes in the spectrum of light which has interacted with body tissue and/or fluid can be used to estimate the molecular composition of that body tissue and/or fluid.

In an example, a wearable device can have a spectroscopic sensor. In an example, a biometric sensor of a system can be a spectroscopic sensor, including a light emitter and light receiver, which collects light energy data which then is analyzed using spectroscopic analysis in order to measure the chemical composition of body tissue, organs, and/or fluid. In an example, a biometric sensor of a system can be a spectroscopic sensor, including a light emitter and light receiver, which collects light energy data which then is analyzed using spectroscopic analysis in order to monitor changes in the chemical composition of body tissue, organs, and/or fluid. In an example, the biometric sensor of a system can be a spectroscopic sensor, including a light emitter and light receiver, which collects light energy data which then is analyzed using spectroscopic analysis in order to measure the physical configuration of body tissue, organs, and/or fluid.

In an example, a wearable device can have a spectrometry sensor. In an example, a wearable device can have a backscattering spectrometry sensor. In an example, a wearable device can have a mass spectrometry sensor. In an example, a light emitter and a light receiver together can comprise a sensor selected from the group consisting of: backscattering spectrometry sensor, infrared spectroscopy sensor, ion mobility spectroscopic sensor, mass spectrometry sensor, Near Infrared spectroscopy sensor (NIS), Raman spectroscopy sensor, spectrometry sensor, spectrophotometer, spectroscopy sensor, ultraviolet spectroscopy sensor, and white light spectroscopy sensor.

In an example, a wearable device can have a spectrometric sensor. In an example, a wearable device can have a spectrophotometer. In an example, a light emitter and a light received can be collectively referred to as a spectroscopic (or spectroscopy) sensor. In an example, a system can collect data concerning changes in the spectral distribution, intensity, and/or polarization of light that has been reflected from, passed through, and/or scattered by skin, epidermis, blood, blood vessels, intercellular fluid, lymph, muscle tissue, nerve tissue, or other body tissue or fluids. In an example, a system can collect data concerning changes in the spectral distribution, intensity, and/or polarization of light that has been reflected from, passed through, and/or scattered by body tissue, organs, and/or fluid.

In an example, a wearable device can have a spectral analysis sensor. In an example, a system can collect light energy data which is used to measure changes in the chemical composition and/or physical configuration of skin, blood, blood vessels, intercellular fluid, and/or muscles based on how the spectral distribution of light is changed by being reflected from, or passing through, the skin, blood, blood vessels, intercellular fluid, and/or muscles. In an example, the spectral distribution of reflected and/or transmitted light can be analyzed to infer the chemical composition and/or physical configuration of the body tissue, organs, and/or fluid through which the light has passed. In an example, a first light emitter can emit light energy with a first light wavelength (or wavelength range or spectral distribution) and a second light emitter can simultaneously emit light energy with a second light wavelength (or wavelength range or spectral distribution) during the same time period.

In an example, a wearable device can have a Raman spectroscopy sensor. In an example, a wearable device can have a photochemical sensor. In an example, a wearable device can have a photocell. In an example, a wearable device can have a liquid chromatography sensor. In an example, a wearable device can have a gas chromatography sensor. In an example, a wearable device can have a fluorescence sensor. In an example, a wearable device can have a colorimetric sensor. In an example, a wearable device can have a chemiluminescence sensor. In an example, a wearable device can have a first light emitter which emits light with a wavelength within the range of 600 to 700 nm and second light emitter which emits light with a wavelength within the range of 850 to 950 nm.

In an example, one or more light emitters can be selected from the following types of light emitters: arc source, blackbody source, coherent light source, incandescent bulb, infrared light emitter, laser, Laser Diode (LD), light Emitting Diode (LED), mercury lamp, microplasma light emitter, multi-wavelength source, Organic light Emitting Diode (OLED), Resonant Cavity light Emitting Diode (RCLED), Superluminescent light Emitting Diode (SLED), ultraviolet light emitter, and tungsten lamp. In an example, a wearable component of a system can include one or more light receivers which are selected from the group consisting of: avalanche photodiode (APD), charge-coupled device (CCD), complementary metal-oxide semiconductor (CMOS), digital camera, field effect transistor, infrared detector, infrared photoconductor, infrared photodiode, light dependent resistor (LDR), light energy sensor, microbolometer, optical detector, optical sensor, photoconductor, photodetector, photodiode, photomultiplier, photoresistor, phototransistor, and spectroscopic sensor.

In an example, the wearable device can have a spectroscopic sensor. In an example, a light emitter and a light receiver which are in optical communication with each other can comprise a spectroscopic sensor. In an example, a light emitter and light receiver together can be referred to as a spectroscopic sensor. In an example, a wearable device can have a coherent light spectroscopic sensor. In an example, a wearable device can have a fiber optic spectroscopic sensor. In an example, a wearable device can have an ambient light spectroscopic sensor. In an example, a wearable device can have an infrared spectroscopic sensor. In an example, a wearable device can have an ultraviolet spectroscopic sensor. In an example, a wearable device of a system can have a laser spectroscopic sensor. In an example, a wearable device of a system can have a near-infrared spectroscopic sensor. In an example, a wearable device of a system can have a visible or white light spectroscopic sensor.

In an example, a first light emitter can emit light energy with a first light wavelength (or wavelength range or spectral distribution) and a second light emitter can simultaneously emit light energy with a second light wavelength (or wavelength range or spectral distribution) during the same time period in order to measure different physiological parameters, analytes, or conditions. In an example, a first light emitter can emit light with a first light wavelength (or wavelength range or spectral distribution) and a second light emitter can simultaneously emit light with a second light wavelength (or wavelength range or spectral distribution) during the same time period. In an example, the first light emitter can emit light with a first wavelength (or spectral distribution) and the second light emitter can emit light with a second wavelength (or spectral distribution).

In an example, a first subset of spectroscopic sensors in a plurality of spectroscopic sensors can direct light beams toward a person's body at a first angle and a second subset of spectroscopic sensors in the plurality of spectroscopic sensors can direct light beams toward the person's body at a second angle. In an example, a first subset of spectroscopic sensors in a plurality of spectroscopic sensors can direct light beams with a first orientation or degree of polarization or coherence toward a person's body and a second subset of spectroscopic sensors in the plurality of spectroscopic sensors can direct light beams with a second orientation or degree of polarization or coherence toward the person's body.

In an example, a light emitter can emit light energy with a first light wavelength (or wavelength range or spectral distribution) during a first time period and can emit light energy with a second light wavelength (or wavelength range or spectral distribution) during a second time period in response to changing physiological conditions. In an example, a light emitter can emit light energy with a first light wavelength (or wavelength range or spectral distribution) during a first time period and can emit light energy with a second light wavelength (or wavelength range or spectral distribution) during a second time period in response to changing biometric results.

In an example, a light emitter can emit light energy with a first light wavelength (or wavelength range or spectral distribution) during a first time period and can emit light energy with a second light wavelength (or wavelength range or spectral distribution) during a second time period in order to measure different physiological parameters, analytes, or conditions. In an example, a light emitter can emit light with a first light wavelength (or wavelength range or spectral distribution) during a first time period and can emit light with a second light wavelength (or wavelength range or spectral distribution) during a second time period in response to changing physiological conditions.

In an example, a system or method can comprise a first light emitter which emits light with a first light wavelength (or wavelength range or spectral distribution) and a second light emitter which simultaneously emits light with a second light wavelength (or wavelength range or spectral distribution) during the same time period in order to measure different physiological parameters, analytes, or conditions. In an example, a light receiver can automatically cycle through light energy emissions with a variety of wavelengths (or wavelength ranges or spectral distributions) during a different time periods in order to measure different physiological parameters, analytes, or conditions. In an example, one or more light filters can partially absorb and/or block one or more selected light wavelengths, wavelength ranges, frequencies, and/or frequency ranges. In an example, a light emitter can emit light with a first light wavelength (or wavelength range or spectral distribution) during a first time period and can emit light with a second light wavelength (or wavelength range or spectral distribution) during a second time period in order to measure different physiological parameters, analytes, or conditions.

In an example, changes, gaps, and/or shifts in selected frequencies in the spectrum of ambient light due to interaction with a person's body tissue and/or fluid can be analyzed to monitor changes in the chemical composition of the person's body tissue and/or fluid. In an example, different spectroscopic sensors in a plurality of spectroscopic sensors can differ with respect to the color, frequency, and/or spectrum of light beams which they direct toward a person's body. In an example, the frequency, color, and/or spectrum of a beam of light emitted from a light emitter can be changed over time to create a chronological sequence of beams of light with different frequencies, colors, and/or spectrums. In an example, the frequency, color, and/or spectrum of light emitted from the light emitter can be adjusted in order to more accurately measure a biometric indicator value in the body.

In an example, different light emitters can differ with respect to the color, frequency, and/or spectrum of light beams which they direct toward a person's body. In an example, a first subset of light emitters can direct light beams with a first color, frequency, and/or spectrum toward a person's body and a second subset of light emitters can direct light beams with a second color, frequency, and/or spectrum toward the person's body. In an example, a system can further comprise one or more optical filters or lenses which change the frequency, color, and/or spectrum of light emitted by a light emitter. In an example, data from a spectroscopic sensor can be analyzed to determine how the spectrum of ambient light has been changed by reflection from, or passage through, body tissue, organs, and/or fluid.

In an example, this light sensor can be a spectroscopic sensor (which can alternatively be called a spectroscopy sensor). In an example, data from a spectroscopic sensor can be analyzed to determine how the spectrum of directed light has been changed by reflection from, or passage through, the person's body tissue and/or fluid. In an example, a spectroscopic sensor can comprise a light emitter (e.g. light source) and a light receiver (e.g. photodetector) on the same side (e.g. the ventral or dorsal side) of a body member, wherein the light receiver receives light from the light emitter after that light has been reflected by body tissue and/or fluid. In an example, a spectroscopic sensor can comprise both a light emitter and a light receiver if it the light receiver receives light which has been emitted by the light emitter and then reflected from (or passed through) body tissue, organs, and/or fluid.

In an example, a biometric sensor of a system can be integrated into an armlet, bangle, bracelet, cuff, fitness band, gauntlet, sleeve, smart watch, strap, watch, or wrist band. In an example, a light emitter can be part of an arcuate band. In an example, a light receiver can be part of an arcuate band. In an example, a light receiver can be part of a housing which is held on a person's body by an arcuate band. In an example, a plurality of light emitters and receivers can be distributed around (at least half of) the circumference of an ankle band or smart sock. In an example, this device can comprise a two-dimensional array of emitters and receivers which is part of a wearable arcuate band or one or more segments (or housings) which are attached to a wearable arcuate band.

In an example, a light emitter and a light receiver can be part of a housing which is held on a person's body by an arcuate band. In an example, a light emitter can be part of a housing which is held on a person's body by an arcuate band. In an example, a light receiver can be part of a housing which is held on a person's body by an arcuate band. In an example, this device can comprise an array of emitters and receivers which is part of a wearable arcuate band or one or more segments (or housings) which are attached to a wearable arcuate band. In an example, this device can comprise a three-dimensionally stacked array of emitters and receivers which is part of a wearable arcuate band or one or more segments (or housings) which are attached to a wearable arcuate band.

In an example, a system can comprise an optical sensor. In an example, a first light emitter in an optical sensor can emit light with a first light coherence, polarization, and/or phase and a second light emitter in the optical sensor can emit light with a second light coherence, polarization, and/or phase. In an example, a light emitter in an optical sensor can be automatically rotated by an actuator. In an example, the angle of light emitted from a light emitter can be adjusted automatically to maintain accurate measurement of a biometric indicator value in a person's body even if the system shifts and/or moves relative to the person's body surface. In an example, a wearable photoplethysmography (PPG) device can further include one or more light guides (such as lenses, mirrors, or prisms) which direct the paths of light rays between the light emitter and a person's body or between the person's body and the light receiver.

In an example, a system can comprise sensors in the housing (e.g. the primary display housing) of a smart watch, around the band of a smart watch, or both. In an example, a wearable component of a system can have a circumferential array of light receivers which spans between 25% and 100% of the cross-sectional perimeter circumference of a part of the body (e.g. finger, wrist, arm, ankle, or leg) to which the system is attached. In an example, a wearable component of a system can have a circumferential array of light emitters which spans between 25% and 100% of the cross-sectional perimeter circumference of a part of the body (e.g. finger, wrist, arm, ankle, or leg) to which the system is attached. In an example, wearable photoplethysmography (PPG) device can comprise a circumferential array of light emitters which are distributed around the circumference of a body member such as a finger, wrist, or arm.

In an example, a system or method can include a biometric sensor which is located in a primary housing of a wrist-worn device (such as a smart watch), wherein the primary housing is worn on the dorsal side of a person's wrist. In an example, data concerning one or more biometric indicators can be obtained from sensors on a wrist band and/or arm band which is worn by the person. In an example, data concerning one or more biometric indicators can be obtained from sensors on a smart watch which is worn by the person. In an example, data concerning one or more biometric indicators can be obtained from sensors on a watch band (e.g. watch band with a plurality of sensors) which is worn by the person. In an example, wearable photoplethysmography (PPG) device can comprise a circumferential array of light emitters and light receivers which are distributed around the circumference of a body member such as a finger, wrist, or arm.

In an example, a wearable component of a system can include one or more mirrors. In an example, a beam of light can: be emitted by the light emitter along a first vector; pass through the first (transmissive) side of an angled one-way mirror; hit body tissue; reflect back from the body tissue; reflect off the second (reflective) side of the angled one-way mirror; and enter the light receiver along a second vector which is perpendicular to the first vector. In an example, a beam of light emitted by a light emitter can be automatically moved by using an actuator to automatically move a light reflector (such as a mirror) from which this beam is reflected. In an example, a system can comprise an array of moving micromirrors. In an example, the beam of light emitted by a light emitter can be automatically moved by using an actuator to automatically rotate, tilt, raise, or lower a light reflector (such as a mirror) from which this beam is reflected.

In an example, a wearable component of a system can include one or more light-blocking layers, coatings, claddings, and/or components. In an example, a wearable component of a system can include one or more light filters. In an example, a wearable component of a system can include one or more lenses. In an example, a wearable component of a system can include one or more body motion and/or configuration sensors. In an example, a wearable component of a system can include an array of light receivers. In an example, a wearable component of a system can include a lens which selectively refracts and/or focuses light.

In an example, a wearable device of a system can be worn on a person's foot. In an example, a wearable device of a system can be worn on a person's ankle or foot. In an example, a wearable component of a system can be configured to be worn on a person's leg and/or foot. In an example, a wearable device can be an ankle band, smart sock, foot pad, or toe ring. In an example, light emitters and receivers on a wearable device can be configured to be distributed around (at least half of) the circumference of a person's finger, wrist, arm, ankle, or leg. In an example, an array, grid, and/or matrix of two or more light emitters can span between 50% and 100% of the cross-sectional circumference of a part of a person's body such as a finger, wrist, arm, ankle, or leg.

In an example, a wearable device of a system can be worn on a person's finger. In an example, the wearable component of the system can be a finger ring. In an example, pairs of light emitters and light receivers can be distributed around the circumference of a wearable device (such as a finger ring, watch band, wrist band, arm band, or ankle band) such that the light beam from at least one light emitter is substantially perpendicular to the proximal surface of a person's body regardless of rotation and/or shifting of the wearable device. In an example, a plurality of light emitters and receivers can comprise a circumferential array of light emitters and receivers around (at least half of the circumference of) a person's finger, wrist, arm, ankle, or toe. In an example, an array, grid, and/or matrix of (alternating) light emitters and receivers can span between 25% and 50% of the circumference of a part of a person's body such as a wrist, arm, finger, ankle, or leg.

In an example, light emitters in a system can be lasers. In an example, a light emitter can be a near infrared laser. In an example, a light emitter can be a red-light laser. In an example, a wearable device can have both a red-light laser and a green-light laser. In an example, a light emitter emit green light and/or be a green-light laser. In an example, a light emitter can emit coherent and/or laser light. In an example, a light emitter can emit white light and/or be a white-light laser.

In an example, this device can further comprise one or more other types of biometric or environmental sensors in addition to the primary emitters and receivers discussed above. In an example, the power and/or intensity of a beam of light emitted from a light emitter can be changed in response to specific environmental conditions (e.g. temperature or humidity) and/or specific activities in which the person wearing a system is engaged (e.g. high level of movement, eating, sleeping, etc.) in order to more accurately measure a biometric indicator value in the body. In an example, the projection angle of a beam of light emitted from a light emitter can be changed in response to specific environmental conditions (e.g. temperature or humidity) and/or specific activities in which the person wearing a system is engaged (e.g. high level of movement, eating, sleeping, etc.) in order to more accurately measure a biometric indicator value in the body.

In an example, a plurality of light emitters can be configured in a polygonal array around a light receiver. In an example, a plurality of light emitters can be configured in a circular or other arcuate array around a light receiver. In an example, a plurality of light receivers can be configured in a circular or other arcuate array around a light emitter. In an example, a plurality of light receivers can be configured in a polygonal array around a light emitter.

In an example, a system can comprise an array, grid, and/or matrix of two or more light receivers along a proximal-to-distal axis. In an example, a system can comprise an array, grid, and/or matrix of two or more light receivers along a circumferential axis. In an example, a system can comprise an array, grid, and/or matrix of (alternating) light emitters and receivers along a proximal-to-distal axis. In an example, a system can comprise an array, grid, and/or matrix of two or more light emitters along a proximal-to-distal axis. In an example, an array of emitters and/or receivers can have a circumferential axis and a proximal-to-distal axis.

Stress is associated with changes in electromagnetic brain activity. Sleep and/or different stages of sleep are associated with changes in electromagnetic brain activity. Relaxation is associated with changes in electromagnetic brain activity. In an example, a pattern of electromagnetic brain activity can be a change in activity in a specific area of a person's brain as measured from one or more specific sensor locations on the person's head. In an example, this pattern can be a change in electromagnetic brain activity measured from one location or channel relative to electromagnetic brain activity measured from one or more different locations or channels. In an example, this pattern can be a transient pattern which is recorded from one or more locations. In an example, this pattern can be the start of a repeating pattern which is recorded from one or more locations. In an example, this pattern can be a change in an ongoing repeating pattern which is recorded from one or more locations.

In an example, body motion or exercise level can be measured from multiple locations on the person's body. In an example, body lactate and/or lactic acid level can be measured from multiple locations on the person's body. In an example, body hydration level can be measured from multiple locations on the person's body. In an example, body glucose level can be measured from multiple locations on a person's body. In an example, body carbon dioxide levels can be measured from multiple locations on a person's body.

In an example, a closed loop system can adjust the operation of a cardiac pacemaker in response to high heart rate variability in one or more ways selected from the group consisting of: change in heart electromagnetic stimulation voltage, change in the degree of coordination and/or timing between stimulations to different heart chambers, change in the frequency of heart contraction stimulations, change in the locations on the heart to which electromagnetic energy is delivered, change in the magnitude of heart contraction stimulations, change in the regularity of heart contraction stimulations, delivery of an electromagnetic shock to the heart, and more precise coordination of contraction of different heart chambers.

In an example, a closed loop system can adjust the operation of a cardiac pacemaker in response to abnormal pulsatile blood volume in one or more ways selected from the group consisting of: change in heart electromagnetic stimulation voltage, change in the degree of coordination and/or timing between stimulations to different heart chambers, change in the frequency of heart contraction stimulations, change in the locations on the heart to which electromagnetic energy is delivered, change in the magnitude of heart contraction stimulations, change in the regularity of heart contraction stimulations, delivery of an electromagnetic shock to the heart, and more precise coordination of contraction of different heart chambers.

In an example, a closed loop system can adjust the operation of a cardiac pacemaker in response to abnormal Pulsatile Blood Lag (PBL) in one or more ways selected from the group consisting of: change in heart electromagnetic stimulation voltage, change in the degree of coordination and/or timing between stimulations to different heart chambers, change in the frequency of heart contraction stimulations, change in the locations on the heart to which electromagnetic energy is delivered, change in the magnitude of heart contraction stimulations, change in the regularity of heart contraction stimulations, delivery of an electromagnetic shock to the heart, and more precise coordination of contraction of different heart chambers.

In an example, a method can adjust the operation of a cardiac pacemaker in response to exhaled gas composition in one or more ways selected from the group consisting of: change in heart electromagnetic stimulation voltage, change in the degree of coordination and/or timing between stimulations to different heart chambers, change in the frequency of heart contraction stimulations, change in the locations on the heart to which electromagnetic energy is delivered, change in the magnitude of heart contraction stimulations, change in the regularity of heart contraction stimulations, delivery of an electromagnetic shock to the heart, and more precise coordination of contraction of different heart chambers.

In an example, a system or method can adjust the operation of a cardiac pacemaker in response to an abnormal body hydration level in one or more ways selected from the group consisting of: change in heart electromagnetic stimulation voltage, change in the degree of coordination and/or timing between stimulations to different heart chambers, change in the frequency of heart contraction stimulations, change in the locations on the heart to which electromagnetic energy is delivered, change in the magnitude of heart contraction stimulations, change in the regularity of heart contraction stimulations, delivery of an electromagnetic shock to the heart, and more precise coordination of contraction of different heart chambers.

In an example, one or more operating parameters of a cardiac pacemaker which are adjusted by a system or method can be selected from the group consisting of: increase in heart electromagnetic stimulation voltage; increase in the degree of coordination and/or timing between stimulations to different heart chambers; increase in the frequency of heart contraction stimulations; change in the locations on the heart to which electromagnetic energy is delivered; increase in the magnitude of heart contraction stimulations; increase in the regularity of heart contraction stimulations; and more precise coordination of contraction of different heart chambers.

In an example, a biometric indicator which is measured and managed by a system or method can be body motion or exercise level. In an example, a system can change blood circulation by adjusting the operation of a cardiac pacemaker in response to a high body motion or exercise level. In an example, a closed loop system can adjust the operation of a central (heart-assist) blood pump or a non-central (peripheral) blood pump in response to a high body motion or exercise level in one or more ways selected from the group consisting of: activation of the device to change blood flow; adjusted device pumping volume to change blood flow; adjusted device rotation and/or speed to change blood flow; changed duration of device operation to change blood flow; selective activation of a sub-set of non-central (peripheral) blood pumps to change blood flow in a selected body location.

In an example, a closed loop system can adjust the operation of a central (heart-assist) blood pump or a non-central (peripheral) blood pump in response to troponin in one or more ways selected from the group consisting of: activation of the device to change blood flow; adjusted device pumping volume to change blood flow; adjusted device rotation and/or speed to change blood flow; changed duration of device operation to change blood flow; selective activation of a sub-set of non-central (peripheral) blood pumps to change blood flow in a selected body location. In an example, a plurality of non-central (peripheral) blood pumps can comprise a fluid network of mini-hearts which support a person's heart only to the extent which is needed during a period of cardiac healing and recovery.

In an example, a closed loop system can adjust the operation of a central (heart-assist) blood pump or a non-central (peripheral) blood pump in response to high heart rate variability in one or more ways selected from the group consisting of: activation or deactivation of the pump to change blood flow; change in the duration of pump operation to change blood flow; change in the magnitude of pump undulation, compression, or contraction (depending on type of pump) to change blood flow; change in the speed of a pump's rotation, undulation, compression, or contraction (depending on type of pump) to change blood flow; and selective activation of a sub-set of non-central (peripheral) blood pumps to change blood flow in a selected sub-set of body locations.

In an example, a closed loop system can adjust the operation of a central (heart-assist) blood pump or a non-central (peripheral) blood pump in response to sleep status or stage in one or more ways selected from the group consisting of: activation of the device to change blood flow; adjusted device pumping volume to change blood flow; adjusted device rotation and/or speed to change blood flow; changed duration of device operation to change blood flow; selective activation of a sub-set of non-central (peripheral) blood pumps to change blood flow in a selected body location.

In an example, a closed loop system can adjust the operation of a central (heart-assist) blood pump or a non-central (peripheral) blood pump in response to exhaled gas composition in one or more ways selected from the group consisting of: activation of the device to change blood flow; adjusted device pumping volume to change blood flow; adjusted device rotation and/or speed to change blood flow; changed duration of device operation to change blood flow; selective activation of a sub-set of non-central (peripheral) blood pumps to change blood flow in a selected body location.

In an example, a closed loop system can adjust the operation of a central (heart-assist) blood pump or a non-central (peripheral) blood pump in response to abnormal body glucose level in one or more ways selected from the group consisting of: activation of the device to change blood flow; adjusted device pumping volume to change blood flow; adjusted device rotation and/or speed to change blood flow; changed duration of device operation to change blood flow; and selective activation of a sub-set of non-central (peripheral) blood pumps to change blood flow in a selected body location.

In an example, a system can adjust the operation of a central (heart-assist) blood pump or a non-central (peripheral) blood pump in response to abnormal Pulsatile Blood Volume (PBV), or variation thereof, in one or more ways selected from the group consisting of: activation or deactivation of the pump to change blood flow; change in the duration of pump operation to change blood flow; change in the magnitude of pump undulation, compression, or contraction (depending on type of pump) to change blood flow; change in the speed of a pump's rotation, undulation, compression, or contraction (depending on type of pump) to change blood flow; and selective activation of a sub-set of non-central (peripheral) blood pumps to change blood flow in a selected sub-set of body locations.

In an example, a closed loop system can adjust the operation of a central (heart-assist) blood pump or a non-central (peripheral) blood pump in response to blood pressure in one or more ways selected from the group consisting of: activation of the device to change blood flow; adjusted device pumping volume to change blood flow; adjusted device rotation and/or speed to change blood flow; changed duration of device operation to change blood flow; selective activation of a sub-set of non-central (peripheral) blood pumps to change blood flow in a selected body location.

In an example, a system can include a plurality of biometric sensors at different locations relative to the wearable component and/or at different locations relative to a person's body. Two external sensors at two different locations on body—adjust based on min value between them, or max value between them, or raw difference between then, or timing difference in pulsation between them, of difference in variation. In an example, troponin level can be measured from multiple locations on the person's body. In an example, sleep status or stage can be measured from multiple locations on the person's body. In an example, Pulsatile Blood Volume (PBV) can be measured from multiple locations on a person's body. In an example, Pulsatile Blood Lag (PBL) can be measured from multiple locations on the person's body.

In an example data from wearable sensors can be analyzed using an analytical method selected from the group consisting of: analysis of Variance (ANOVA), artificial Neural Network (ANN), Auto-Regressive (AR) modeling, Bayesian analysis, Bonferroni analysis (BA), Centroid analysis, Chi-Squared analysis, Cluster analysis, Correlation, Covariance, Data Normalization (DN), Decision Tree analysis (DTA), Discrete Fourier transform (DFT), Discriminant analysis (DA), Edgar AI analysis, Carlavian Curve analysis (CCA), and Empirical Mode Decomposition (EMD).

In an example, an electromagnetic energy sensor can collect data concerning changes in the transmission of electromagnetic energy from the emitter to the receiver due to changes in neuromuscular activity. In an example, an electromagnetic energy sensor can be an electromyographic (EMG) sensor. In an example, an electromagnetic energy sensor can be an electromagnetic energy receiver which receives electromagnetic energy which is naturally generated by the electromagnetic activity of body tissue and/or organs. In an example, an electromagnetic energy emitter can be on one side of an ear lobe and an electromagnetic energy receiver can be on the opposite side of the ear lobe.

In an example, a person's skin moisture level can be measured by measuring electromagnetic energy from (and/or passing electromagnetic energy through) body tissue. In an example, a person's tissue impedance level can be measured by measuring electromagnetic energy from (and/or passing electromagnetic energy through) body tissue. In an example, a plurality of electromagnetic energy emitters and receivers can be distributed around (at least half of) the circumference of a finger ring or finger sleeve. In an example, a plurality of electromagnetic muscle activity sensors can be configured to collect data concerning electromagnetic neuromuscular activity. In an example, a system can include an electromyographic (EMG) sensor which is incorporated into an article of clothing or a clothing accessory. In an example, a system can include a plurality of electromyographic (EMG) sensors which are incorporated into an article of clothing or a clothing accessory.

In an example, an electromagnetic energy sensor that collects data concerning neuromuscular activity can be a capacitive sensor or a conductive sensor. In an example, an electromagnetic energy sensor that collects data concerning neuromuscular activity can be a dry electrode or a wet electrode. In an example, an electromagnetic energy sensor can comprise an electromagnetic energy emitter and an electromagnetic energy receiver which are in proximity to a person's muscles. In an example, an electromagnetic energy sensor can comprise an electromagnetic energy emitter and an electromagnetic energy receiver which are in proximity to a person's head.

In an example, one or more body motion and/or configuration sensors can be used to identify whether the person is: walking or running; engaging in a particular type of exercise; playing a particular type of sport; eating; and/or sleeping. In an example, a system or method can optimize cardiac function for physical activity and/or exercise level. In an example, a finger ring, smart watch, smart watch band, wrist band, ankle band, smart sock, ear ring, ear clip, or ear bud can collect data on a biometric indicator selected from the group consisting of: oxygenation level, carbon dioxide level, lactate or lactic acid level, blood pressure, heart rate variability, pulsatile blood volume, pulsatile blood lag, hydration level, respiration rate, exhaled gas composition, body glucose level, troponin level, body motion or exercise level, and sleep status or stage.

In an example, biometric sensors in a system can be selected from the group consisting of electromagnetic energy sensors, blood pressure sensors, and oxygenation sensors. In an example, a closed loop system can adjust the operation of a cardiac pacemaker in response to low body oxygenation in one or more ways selected from the group consisting of: increase in heart electromagnetic stimulation voltage; increase in the degree of coordination and/or timing between stimulations to different heart chambers; increase in the frequency of heart contraction stimulations; change in the locations on the heart to which electromagnetic energy is delivered; increase in the magnitude of heart contraction stimulations; increase in the regularity of heart contraction stimulations; and more precise coordination of contraction of different heart chambers.

In an example, a closed loop system can adjust the operation of a non-central (peripheral) blood pump in response to low body oxygenation in one or more ways selected from the group consisting of: activation of the pump to increase blood flow; increase in the duration of pump operation to increase blood flow; increase in the magnitude of pump undulation, compression, or contraction (depending on type of pump) to increase blood flow; increase in the speed of a pump's rotation, undulation, compression, or contraction (depending on type of pump) to increase blood flow; and selective activation of a sub-set of non-central (peripheral) blood pumps to change blood flow in a selected sub-set of body locations.

A person's body can have regions with high and low blood circulation (and body oxygenation). In an example, body oxygenation levels can be measured from multiple locations on a person's body. One potential advantage is greater accuracy and selectivity in maintaining biometric indicators (such as oxygenation) in different portions of a person's body. This can help to avoid physiological dysfunction and potentially even limb loss due to poor circulation and oxygenation. In an example, a system can include one or more wearable sensors selected from the group consisting of oxygen saturation sensor, SpO2 sensor, and pulse oximeter. In an example, detection of low oxygenation levels in specific portions of the body by wearable sensors can trigger selected increased blood flow to those specific portions.

In an example, an electromagnetic brain activity sensor can be configured to collect data concerning electromagnetic brain activity which is affected by body tissue and/or fluid oxygen levels (or changes in those levels). In an example, a biometric sensor of a system can be integrated into an ear bud, ear hook, ear plug, ear ring, earlobe clip, earphone, earpiece, earring, ear-worn Bluetooth communication device, electroencephalographic (EEG) sensor, oximeter, headphone, headset, or hearing aid. In an example, abnormal body tissue and/or fluid oxygen levels (or changes in those levels) can trigger changes in repeating patterns and/or transient patterns of electromagnetic brain activity. In an example, a wearable device can have a spectroscopic oximeter. In an example, an electromagnetic brain activity sensor can be configured to collect data concerning electromagnetic brain activity which is affected by body tissue and/or fluid oxygen levels (or changes in those levels). In an example, a system can adjust parameters of cardiac functioning in response to low oxygen levels detected by a wearable electromagnetic brain activity sensor.

In an example, an optical biometric sensor of a system (such as a spectroscopic sensor) can be configured to collect data concerning one or more biometric indicators or conditions selected from the group consisting of: exercise level, exhaled breath volume, eye movement, galvanometric response, glucose level, GSR data, heart arrhythmia, heart rate, heart rate variability (HRV), heartbeat irregularity, hemoglobin level, hormone level, hydration, hypertension, inhaled breath volume, interstitial glucose level, intracranial pressure, iron level, jaw motion, joint angle, lactate level, lactic acid, limb acceleration, limb configuration, lipid levels, magnesium level, maximum volume of oxygen consumption, metabolism, methemoglobin level, muscle tension, myoglobin level, neurological activity level, nickel level, nitrogen level, overall body activity level, oxygen level, oxygen saturation, partial pressure of carbon dioxide, partial pressure of oxygen, perspiration level or rate, pH level, pheromone level, phosphorus level, potassium level, pressure level, protein levels, pulse, QRS, respiration rate, respiration volume, resting heart rate, skin humidity, skin impedance, skin resistance, sodium level, sound level, SpCO2, swallowing rate, sweating rate or level, tachycardia, tearing, temperature (core body), temperature (skin), thyroid stimulating hormone (TSH) level, tissue impedance, tissue oxygen level, triglyceride level, troponin level, urea nitrogen level, VO2 max, and body water level.

In an example, a method can comprise receiving biometric data from a pressure sensor, blood pressure sensor, and/or blood pressure cuff. In an example, a method can comprise receiving biometric data from a humidity and/or moisture sensor. In an example, a method can comprise receiving biometric data from a chemical sensor. In an example, a method can comprise receiving biometric data from a camera. In an example, a method can comprise receiving biometric data from a blood glucose sensor.

In an example, a system can further comprise one or more components selected from the group consisting of: pollution sensor, thermal energy sensor, blood pressure sensor, conductive fabric, energy transducer to generate energy from body motion or kinetic energy, keypad, power source, thermistor, blood reservoir, conductivity sensor, energy transducer to generate energy from body thermal energy, magnetic field sensor, pressure sensor, thermocouple, buzzer, display screen, global positioning system (GPS), mobile phone, and speaker.

In an example, a system can comprise one or more power sources which supply power to the biometric sensor and the data processor. In an example, a power source can be a battery. In an example, this device can further comprise an energy source which powers an emitter, a receiver, a data processor, and/or a data transmitter. In an example, a system can include a wireless data transmitter and/or receiver.

In an example, a closed loop system can adjust the operation of a central (heart-assist) blood pump or a non-central (peripheral) blood pump in response to a high body carbon dioxide level in one or more ways selected from the group consisting of: activation of the pump to increase blood flow; increase in the duration of pump operation to increase blood flow; increase in the magnitude of pump undulation, compression, or contraction (depending on type of pump) to increase blood flow; and increase in the speed of a pump's rotation, undulation, compression, or contraction (depending on type of pump) to increase blood flow.

In an example, a system or method can comprise adjusting the flow rate of a blood pump and/or circulatory assist device based on the values of one or more biometric indicators. In an example, a system or method can comprise gradually increasing or decreasing the flow rate of a blood pump and/or circulatory assist device based on the values of one or more biometric indicators. In an example, a system or method can comprise adjusting the rotational speed of an impellor in a blood pump and/or circulatory assist device based on the values of one or more biometric indicators. In an example, a system or method can comprise adjusting the rotational speed of an impellor in a blood pump and/or circulatory assist device. In an example, a system or method can comprise gradually increasing or decreasing the flow rate of a blood pump and/or circulatory assist device. In an example, a system or method can comprise adjusting the maximum pressure or minimum pressure of a blood pump and/or circulatory assist device.

In an example, a closed loop system can adjust the operation of a central (heart-assist) blood pump or a non-central (peripheral) blood pump in response to a high body lactate and/or lactic acid level in one or more ways selected from the group consisting of: activation of the pump to increase blood flow; increase in the duration of pump operation to increase blood flow; increase in the magnitude of pump undulation, compression, or contraction (depending on type of pump) to increase blood flow; and increase in the speed of a pump's rotation, undulation, compression, or contraction (depending on type of pump) to increase blood flow.

In an example, this method can comprise using machine learning and/or artificial intelligence to adjust the rotational speed of an impellor in a blood pump and/or circulatory assist device based on the values of one or more biometric indicators. In an example, this method can comprise using machine learning and/or artificial intelligence to adjust the operating parameters of a Ventricular Assist Device (VAD). In an example, this method can comprise using machine learning and/or artificial intelligence to adjust the flow rate of a blood pump and/or circulatory assist device based on the values of one or more biometric indicators. In an example, this method can comprise adjusting the operating parameters of an artificial heart. In an example, this method can comprise using machine learning and/or artificial intelligence to adjust the operating parameters of an artificial heart. In an example, this method can comprise using machine learning and/or artificial intelligence to adjust the operating parameters of a mechanical circulatory support device.

In an example, a blood pump can have a first operational mode in which it increases blood flow above normal flow levels and a second operational mode in which it decreases blood flow below normal flow levels. In an example, a blood pump can have a first end which is connected to an upstream portion of a blood vessel, a second end which is connected to a downstream portion of a blood vessel, and a blood-flow-increasing mechanism located between the two ends. In an example, a blood pump can further comprise one or more moving members which increase blood flow by frictionally engaging blood and/or by entraining native blood flow. In an example, a blood pump can be designed to produce hemodynamic patterns that minimize thrombogenesis. In an example, a blood pump can be structurally designed to avoid low-flow areas that can cause thrombogenesis.

In an example, a method can comprise adjusting the operating parameters of an axial blood flow pump. In an example, a method can comprise adjusting the operating parameters of a Ventricular Assist Device (VAD). In an example, a method can comprise adjusting the operating parameters of a mechanical circulatory support device. In an example, a method can comprise adjusting the operating parameters of a Left Ventricular Assistive Device (LVAD). In an example, a method can comprise adjusting the operating parameters of a circulatory assist device. In an example, a method can comprise adjusting the operating parameters of a centrifugal blood flow pump.

In an example, a non-central (peripheral) blood pump can be spliced into a person's vasculature in series with a natural blood vessel in order to help pump blood to a selected peripheral portion of a person's body. In an example, a non-central (peripheral) blood pump can be endovascularly inserted and then expanded within a peripheral blood vessel in order to help pump blood to a selected peripheral portion of a person's body. In an example, a non-central (peripheral) blood pump which is spliced into a person's vasculature replaces a segment of a natural blood vessel. In an example, a non-central (peripheral) blood pump can be a pulsatile flow pump. In an example, a non-central (peripheral) blood pump can be a continuous flow pump.

In an example, this method can comprise using machine learning and/or artificial intelligence to adjust the operating parameters of an axial blood flow pump. In an example, this method can comprise using machine learning and/or artificial intelligence to adjust the operating parameters of a centrifugal blood flow pump. In an example, this method can comprise using machine learning and/or artificial intelligence to adjust the operating parameters of a circulatory assist device. In an example, this method can comprise using machine learning and/or artificial intelligence to adjust the maximum pressure or minimum pressure of a blood pump and/or circulatory assist device based on the values of one or more biometric indicators.

In an example, a biometric indicator which is measured and managed by a system can be body lactate and/or lactic acid level. In an example, a biometric indicator which is measured and managed by a system can be troponin level. In an example, a biometric indicator which is measured and managed by a system can be the glucose level in a person's body tissue and/or fluid. In an example, a biometric indicator which is measured and managed by a system can be exhaled gas composition.

In an example, a system or method can include receiving data from one or more biochemical and/or biologic sensors selected from the group consisting of: amino acid sensor, antibody-based receptor, artificial olfactory sensor, artificial taste bud, biochemical sensor, biological cell sensor, biological sensor, biomimetic sensor, chemical sensor, chemiresistor, chemoreceptor, and cholesterol sensor.

In an example, the value of a biometric indicator can be considered abnormal when it is at least Y % higher than the maximum value in a benchmark range of values, wherein Y % is selected from within a range of 10% to 50%. In an example, a system can adjust parameters of cardiac functioning in response to abnormal water levels which are detected by a wearable biometric sensor (such as a wearable spectroscopic sensor). In an example, a system can adjust parameters of cardiac functioning in response to abnormal glucose levels detected by a wearable electromagnetic brain activity sensor. Even if changes in cardiac function do not change glucose levels, such changes may help the body better cope with abnormal glucose levels without long-term adverse effects.

In an example, a pattern of electromagnetic brain activity which is selected to trigger adjustment of cardiac function can be identified using one or more analytical methods which are selected from the group consisting of: Analysis of Variance (ANOVA), artificial Neural Network (ANN), Auto-Regressive (AR) Modeling, Bayesian Analysis, Bonferroni Analysis (BA), Centroid Analysis, Chi-Squared Analysis, Cluster Analysis, Correlation, Covariance, Data Normalization (DN), Decision Tree Analysis (DTA), Discrete Fourier transform (DFT), Discriminant Analysis (DA), Edgar AI Analysis, Empirical Mode Decomposition (EMD), Factor Analysis (FA), Fast Fourier Transform (FFT), Feature Vector Analysis (FVA), Fisher Linear Discriminant, Fourier Transformation (FT) method, Fuzzy Logic (FL) Modeling, Gaussian Model (GM), Generalized Auto-Regressive Conditional Heteroscedasticity (GARCH) Modeling, Hidden Markov Model (HMM), Independent Components Analysis (ICA), Inter-Band Power Ratio, Inter-Channel Power Ratio, Inter-Montage Power Mean, Inter-Montage Ratio, Kalman Filter (KF), Kernel Estimation, Laplacian Filter, Laplacian Montage Analysis, Least Squares Estimation, Linear Regression, Linear Transform, Logit Model, machine learning (ML), Markov Model, Maximum Entropy Modeling, Maximum Likelihood, Mean Power, Multi-Band Covariance Analysis, Multi-Channel Covariance Analysis, Multivariate Linear Regression, Multivariate Logit, Multivariate Regression, Naive Bayes Classifier, Neural Network, Non-Linear Programming, Non-negative Matrix Factorization (NMF), Power spectral Density, Power spectrum Analysis, Principal Components Analysis (PCA), Probit Model, Quadratic Minimum Distance Classifier, Random Forest (RF), Random Forest Analysis (RFA), Regression Model, Signal Amplitude (SA), Signal Averaging, Signal Decomposition, Sine Wave Compositing, Singular Value Decomposition (SVD), Spline function, Support Vector and/or Machine (SVM), Time Domain Analysis, Time Frequency Analysis, Time Series Model, Trained Bayes Classifier, Variance, Waveform Identification, Wavelet Analysis, and Wavelet Transformation.

In an example, a system can adjust parameters of cardiac functioning in response to abnormal values of one or more of these biometric indicators or conditions as detected by a wearable biometric sensor (such as a wearable spectroscopic sensor). In an example, a wearable device can have a spectroscopic sensor which measures a person's blood pressure. In an example, a system can adjust parameters of cardiac functioning in response to an abnormal blood pressure level which is detected by a wearable biometric sensor (such as a wearable spectroscopic sensor). In an example, a system can adjust parameters of cardiac functioning in response to high lactate levels which are detected by a wearable biometric sensor (such as a wearable spectroscopic sensor). In an example, a system can adjust parameters of cardiac functioning in response to abnormal glucose levels which are detected by a wearable biometric sensor (such as a wearable spectroscopic sensor).

In an example, a system or method can adjust parameters of cardiac functioning in response to abnormal values of one or more of these biometric indicators or conditions as detected by a wearable biometric sensor (such as a wearable spectroscopic sensor). In an example, a wearable device can have a spectroscopic sensor or electromagnetic energy sensor which measures exhaled gas composition. In an example, a system can adjust parameters of cardiac functioning in response to high carbon dioxide levels which are detected by a wearable biometric sensor (such as a wearable spectroscopic sensor). In an example, the wearable device can have a spectroscopic sensor or electromagnetic energy sensor which measures the level of troponin in a person's body tissue and/or fluid. In an example, a system can adjust parameters of cardiac functioning in response to troponin which is detected by a wearable biometric sensor (such as a wearable spectroscopic sensor).

In an example, a system can further comprise one or more components selected from the group consisting of: electroencephalographic (EEG) sensor, heart rate variability sensor, one-way valve, blood volume sensor, control unit, energy transmitted through inductively-coupled coils, magnetometer, pulse oximetry sensor, and thrombus-catching net. In an example, biometric sensors can be spectroscopic sensors or electromagnetic energy sensors. In an example, a system can comprise wearable resistive sensors which measure a person's blood pressure. In an example, a system can include one or more wearable sensors selected from the group consisting of a microphone, a heart sound sensor, and an ultrasound sensor.

In an example, a wearable device can have a sensor which is in optical communication with body tissue, fluid, and/or gas selected from the group consisting of: blood, interstitial fluid, lymphatic fluid, sweat, tears, aqueous humour, saliva, exhaled gas, capillaries, blood vessels, skin, fatty tissue, muscles, and nerves. In an example, a wearable device can have a sensor which is in electromagnetic communication with body tissue, fluid, and/or gas which is selected from the group consisting of: blood, interstitial fluid, lymphatic fluid, sweat, tears, aqueous humour, saliva, exhaled gas, capillaries, blood vessels, skin, fatty tissue, muscles, bones, and nerves. In an example, a wearable device can have a spectroscopy sensor or electromagnetic energy sensor which measures the glucose level of a person's body tissue and/or fluid.

In an example, biometric sensors can be spectroscopic sensors. In an example, biometric sensors can be spectroscopic sensors or electromagnetic energy sensors. In an example, biometric sensors can be woven into at least half of the circumference of an ankle band or smart sock. In an example, a system can monitor a person's mean arterial pressure. In an example, a system can monitor a person's peripheral resistance. In an example, a system can monitor for tachycardia or bradycardia.

In an example, a wearable device can be a fitness band, bracelet, bangle with embedded biometric sensors. In an example, a wearable device such as a smart finger ring, a smart watch, a smart wrist band, a smart ear ring, or smart eyewear for collecting data on a biometric indicator can have a spectroscopic sensor. In an example, spectroscopic sensors in a plurality of spectroscopic sensors can be distributed around (at least half of) the circumference of a finger ring, smart watch band, wrist band, arm band, ankle band, or smart sock. In an example, a wearable component of a system can be worn in a manner similar to an armlet, bangle, bracelet, cuff, fitness band, gauntlet, sleeve, smart watch, strap, watch, or wrist band. In an example, a plurality of electromagnetic energy emitters and receivers can be distributed around (at least half of) the circumference of an ankle band or smart sock. In an example, a wearable device can be a waist belt, a chest band, an adhesive patch, or an electronic tattoo.

In an example, one or more electromagnetic energy sensors can be selected from the group consisting of: electroencephalograph (EEG) sensor, electromyographic (EMG) sensor, electrocardiographic (ECG) sensor, skin and/or tissue impedance sensor, and skin and/or tissue resistance sensor. In an example, a system and/or method can further comprise one or more components selected from the group consisting of: wireless data receiver, buttons, digital memory, gesture recognition interface, microprocessor, sound-emitting member, wireless communication module, amino acid sensor, cell phone, electromagnetic conductivity sensor, home electronics portal, oximetry sensor, AR eyewear, chemical sensor, electromagnetic energy emitter, home thermostat, pH level sensor, action potential sensor, caloric intake monitor, glucose sensor, motion sensor, spectrophotometer, altitude sensor, capnography sensor, electrocardiographic (ECG) sensor, and Hall-effect sensor.

In an example data from wearable sensors can be analyzed using an analytical method selected from the group consisting of: Multivariate Logit, Multivariate Regression, Naive Bayes Classifier, Neural Network, Non-Linear Programming, Non-negative Matrix Factorization (NMF), Power spectral Density, Power spectrum analysis, Principal Components analysis (PCA), Probit model, and Quadratic Minimum Distance Classifier. In an example data from wearable sensors can be analyzed using an analytical method selected from the group consisting of: Random Forest (RF), Random Forest analysis (RFA), Regression model, Signal Amplitude (SA), Signal Averaging, Signal Decomposition, Sine Wave Compositing, Singular Value Decomposition (SVD), Spline function, Support Vector and/or Machine (SVM), Time Domain analysis, Time Frequency analysis, Time Series model, Trained Bayes Classifier, Variance, Waveform Identification, Wavelet analysis, and Wavelet Transformation.

In an example, a plurality of spectroscopic sensors can be incorporated into the band of a smart watch (or wrist band) or fitness band. In an example, a plurality of spectroscopic sensors can comprise a circumferential array of spectroscopic sensors around (at least half of the circumference of) a person's finger, wrist, arm, ankle, or toe. In an example, a plurality of spectroscopic sensors can comprise a cylindrical matrix or grid of spectroscopic sensors. In an example, a plurality of spectroscopic sensors can comprise a ring or circle of spectroscopic sensors around (at least half of the circumference of) a person's finger, wrist, arm, ankle, or toe. In an example, spectroscopic sensors in a plurality of spectroscopic sensors can be distributed around (at least half of) the circumference of a wearable device which encircles a portion of a person's body.

In an example, a system can comprise a wearable sensor embodied in a smart sock or shoe. In an example, a system can comprise a wearable sensor embodied in a finger ring, finger sleeve, finger nail attachment, or glove. In an example, a system can comprise a wearable sensor embodied in a bra, an undershirt, or a underpants. In an example, a system can comprise a wearable sensor embodied in a short-sleeve shirt or a long-sleeve shirt. In an example, a system can comprise a wearable sensor embodied in a button, snap, or zipper.

In an example, a system can comprise wearable capacitive sensors which measure a person's blood pressure. In an example, a system can include one or more wearable sensors selected from the group consisting of pressure sensor, blood pressure sensor, and blood pressure cuff. In an example, a system can include wearable blood pressure sensors which comprise a plurality of expandable (e.g. inflatable) chambers; and pressure sensors. In an example, a system can include wearable blood pressure sensors which comprise a plurality of expandable (e.g. inflatable) chambers; and strain sensors.

In an example, data concerning one or more biometric indicators can be obtained from sensors on a necklace which is worn by the person. In an example, data concerning one or more biometric indicators can be obtained from sensors on a finger ring which is worn by the person. In an example, data concerning one or more biometric indicators can be obtained from sensors on a bra (e.g. smart bra with imbedded sensors) which is worn by the person. In an example, data concerning one or more biometric indicators can be obtained from sensors on a belt which is worn by the person.

In an example, one or more biometric sensors can be held by a wearable component. In an example, an array of biometric sensors can span the entire circumference of the portion of the person's body to which the wearable device is attached. In an example, the array of biometric sensors can span at least half of the circumference of the portion of the person's body to which the wearable device is attached. In an example, the arcuate array of biometric sensors can span some or all of the circumference of the portion of the person's body to which the wearable device is attached to accommodate variable placement, rotation, and/or shifting of the device.

In an example, a method or system can record and analyze differences in the timing of blood pulsation between different locations on a person's body. In an example, a method or system can record and analyze differences in the timing of blood pulsation between different peripheral locations on a person's body. In an example, a method or system can record and analyze differences in the timing of blood pulsation measured from sensors on wearable devices which are on different locations on a person's body. In an example, a method or system can record and analyze differences in the timing of blood pulsation measured from sensors on different extremities of a person's body.

In an example, a method or system can adjust the operating parameters of an implanted medical device based on differences in the timing of blood pulsation between different locations on a person's body. In an example, a method or system can adjust the operating parameters of an implanted medical device based on differences in the timing of blood pulsation between different peripheral locations on a person's body. In an example, a method or system can adjust the operating parameters of an implanted medical device based on differences in the timing of blood pulsation measured from sensors on wearable devices which are on different locations on a person's body. In an example, a method or system can adjust the operating parameters of an implanted medical device based on differences in the timing of blood pulsation measured from sensors on different extremities of a person's body.

In an example, a method or system can record and analyze differences in tissue oxygenation between different locations on a person's body. In an example, a method or system can record and analyze differences in tissue oxygenation between different peripheral locations on a person's body. In an example, a method or system can record and analyze differences in tissue oxygenation measured from sensors on wearable devices which are on different locations on a person's body. In an example, a method or system can record and analyze differences in tissue oxygenation measured from sensors on different extremities of a person's body.

In an example, a method or system can adjust the operating parameters of an implanted medical device based on differences in tissue oxygenation between different locations on a person's body. In an example, a method or system can adjust the operating parameters of an implanted medical device based on differences in tissue oxygenation between different peripheral locations on a person's body. In an example, a method or system can adjust the operating parameters of an implanted medical device based on differences in tissue oxygenation measured from sensors on wearable devices which are on different locations on a person's body. In an example, a method or system can adjust the operating parameters of an implanted medical device based on differences in tissue oxygenation measured from sensors on different extremities of a person's body.

In an example, a method or system can record and analyze differences in peripheral blood pressure between different locations on a person's body. In an example, a method or system can record and analyze differences in peripheral blood pressure between different peripheral locations on a person's body. In an example, a method or system can record and analyze differences in peripheral blood pressure measured from sensors on wearable devices which are on different locations on a person's body. In an example, a method or system can record and analyze differences in peripheral blood pressure measured from sensors on different extremities of a person's body.

In an example, a method or system can adjust the operating parameters of an implanted medical device based on differences in peripheral blood pressure between different locations on a person's body. In an example, a method or system can adjust the operating parameters of an implanted medical device based on differences in peripheral blood pressure between different peripheral locations on a person's body. In an example, a method or system can adjust the operating parameters of an implanted medical device based on differences in peripheral blood pressure measured from sensors on wearable devices which are on different locations on a person's body. In an example, a method or system can adjust the operating parameters of an implanted medical device based on differences in peripheral blood pressure measured from sensors on different extremities of a person's body.

I claim:

1. A method of managing the operation of an implanted medical device comprising:
using machine learning and/or artificial intelligence to identify the effects of one or more operating parameters of an implanted medical device implanted in a person on values of one or more biometric indicators concerning the person during a first time period; and
using the identified effects to adjust the operating parameters of the implanted medical device to change the values of the one or more biometric indicators in a desired direction and/or by a desired amount during a second time period.

2. The method in claim 1 wherein the implanted medical device is selected from the group consisting of: implanted cardiac pacemaker; implanted cardioverter defibrillator; implanted blood pump; implanted neurostimulator; and implanted drug pump.

3. The method in claim 1 wherein the one or more biometric indicators are selected from the group consisting of: blood or tissue oxygen level; blood pressure; heart rate; blood glucose level; respiration rate;
body posture; body motion; body temperature; chewing or swallowing motion; and brainwave pattern.

4. The method in claim 1 wherein data on the one or more biometric indicators comes from one or more sensors which are selected from the group consisting of: optical sensor; spectroscopic optical sensor;
oximeter; PPG sensor; glucose sensor; inertial motion sensor; strain sensor; moisture sensor;
electromagnetic energy sensor; EEG sensor; and EMG sensor.

5. The method in claim 1 wherein at least some of the data on the one or more biometric indicators comes from one or more sensors on one or more wearable devices what are selected from the group consisting of: smart watch or watch band; wrist band; arm band; bracelet; finger ring; necklace; smart eyewear or an eyewear attachment; smart ear bud or ear ring; smart clothing; and sensor-enabled adhesive patch.

6. The method in claim 1 wherein the operating parameters of the implanted medical device are adjusted based at least in part on the values of the one or more biometric indicators during the second time period.

7. The method in claim 1 wherein the operating parameters of the implanted medical device are adjusted based at least in part on changes over time and/or variation over time in the values of the one or more biometric indicators during the second time period.

8. The method in claim 1 wherein data comes at least partly from a plurality of wearable devices which are worn at different locations on the person's body.

9. The method in claim 8 wherein the operating parameters of the implanted medical device are adjusted based at least in part on differences in values for the one or more biometric indicators between sensors in wearable devices at different locations on the person's body.

10. A method of managing the operation of an implanted medical device comprising:
receiving a first set of data collected during a first time period concerning one or more operating parameters of an implanted medical device which is configured to be implanted in a person;
receiving a second set of data collected during a second time period concerning the values of one or more biometric indicators for the person;
using machine learning and/or artificial intelligence to analyze the first set of data and the second set of data to identify lagged effects of the one or more operating parameters of the implanted medical device on the values of the one or more biometric indicators;
receiving a third set of data collected during a third time period concerning the values of the one or more biometric indicators for the person; and
using the third set of data and the previously-identified lagged effects to determine how to change one or more operating parameters of the implanted medical device in order to change the values of the one or more biometric indicators in a desired direction and/or by a desired amount.

11. The method in claim 10 wherein the implanted medical device is selected from the group consisting of: implanted cardiac pacemaker; implanted cardioverter defibrillator; implanted blood pump; implanted neurostimulator; and implanted drug pump.

12. The method in claim 10 wherein the one or more biometric indicators are selected from the group consisting of: blood or tissue oxygen level; blood pressure; heart rate; blood glucose level; respiration rate;
body posture; body motion; body temperature; chewing or swallowing motion; and brainwave pattern.

13. The method in claim 10 wherein data on the one or more biometric indicators comes from one or more sensors which are selected from the group consisting of: optical sensor; spectroscopic optical sensor;
oximeter; PPG sensor; glucose sensor; inertial motion sensor; strain sensor; moisture sensor;
electromagnetic energy sensor; EEG sensor; and EMG sensor.

14. The method in claim 10 wherein at least some of the data on the one or more biometric indicators comes from one or more sensors on one or more wearable devices what are selected from the group consisting of: smart watch or watch band; wrist band; arm band; bracelet; finger ring; necklace; smart eyewear or an eyewear attachment; smart ear bud or ear ring; smart clothing; and sensor-enabled adhesive patch.

15. The method in claim 10 wherein the operating parameters of the implanted medical device are changed based at least in part on the values of the one or more biometric indicators in the third set of data.

16. The method in claim 10 wherein the operating parameters of the implanted medical device are changed based at least in part on changes over time and/or variation over time in the values of the one or more biometric indicators in the third set of data.

17. The method in claim 10 wherein machine learning and/or artificial intelligence is used to analyze the first set of data and the second set of data to identify lagged effects of changes in the one or more operating parameters of the implanted medical device on changes in the values of the one or more biometric indicators.

18. The method in claim 10 wherein the operating parameters of the implanted medical device are changed based at least in part on the whether the values of the one or more biometric indicators are outside their normal ranges for the person in the third set of data.

19. The method in claim 10 wherein the second set of data is at least partly from a plurality of wearable devices which are worn at different locations on the person's body.

20. The method in claim 19 wherein the operating parameters of the implanted medical device are changed based at least in part on differences in values for the one or more biometric indicators between sensors in wearable devices at different locations on the person's body.
